SEQUENCE LISTING (61 sequences)

SEQ ID NO: 1 - RNA, 28 nt, Artificial Sequence
  tP19 (mortalin-binding)
cucaauggcgaaugcccgccuaauaggg

SEQ ID NO: 2 - RNA, 87 nt, Artificial Sequence
  P19 (mortalin-binding)
gggagacaagaauaaacgcucaauggcgaaugcccgccuaauagggcguuaugacuuguugaguucgacaggaggcucacaacaggc

SEQ ID NO: 3 - RNA, 87 nt, Artificial Sequence
  P1 (mortalin-binding)
gggagacaagaauaaacgcucaaugcgcugaaugcccagccgugaaagcgucgauuuccauccuucgacaggaggcucacaacaggc

SEQ ID NO: 4 - RNA, 87 nt, Artificial Sequence
  P15 (vimentin-binding)
gggagacaagaauaaacgcucaaaguugcggcccaaccguuuaauucagaauagugugaugccuucgacaggaggcucacaacaggc

SEQ ID NO: 5 - RNA, 87 nt, Artificial Sequence
  P11 (HSP90-binding)
gggagacaagaauaaacgcucaaaugauugcccauucgguuaugcuugcgcuuccuaaagagcuucgacaggaggcucacaacaggc

SEQ ID NO: 6 - RNA, 87 nt, Artificial Sequence
  P7 (HSP90-binding)
gggagacaagaauaaacgcucaaggccauguugaaugcccaacuaagcuuugagcuuuggagcuucgacaggaggcucacaacaggc

SEQ ID NO: 7 - RNA, 71 nt, Artificial Sequence
  P6 (HSP90-binding)
gggagacaagaauaaacgcucaacaauggagcguuaaacgugagccauucgacaggaggcucacaacaggc

SEQ ID NO: 8 - RNA, 8 nt, Artificial Sequence
  P19, P1, P7 consensus
gaaugccc

SEQ ID NO: 9 - RNA, 87 nt, Artificial Sequence
  TR14 (TfR-binding)
gggagacaagaauaaacgcucaaugcguucacguuuauucacauuuuugaauugagcaugagcuucgacaggaggcucacaacaggc

SEQ ID NO: 10 - RNA, 87 nt, Artificial Sequence
  TR18 (TfR-binding)
gggagacaagaauaaacgcucaaugcguuuacguuuauucacauuuuugaauugagcaugagcuucgacaggaggcucacaacaggc

SEQ ID NO: 11 - RNA, 43 nt, Artificial Sequence
  Truncated TR14 (TfR-binding)
ggggcucaaugcguucacguuuauucacauuuuugaauugagc

SEQ ID NO: 12 - RNA, 90 nt, Artificial Sequence
  PDR3 (PDGFR-a-binding)
gggagagcggaagcgugcugggccugcucuuuaauaaacccacuuucgaacaucagcguauguccauaacccagaggugauggauccccc

SEQ ID NO: 13 - RNA, 90 nt, Artificial Sequence
  PDR9 (PDGFR-a-binding)
gggagagcggaagcgugcugggccuauugcaucuuucuguuauuuccgaauccgucccgacugucauaacccagaggugauggauccccc

SEQ ID NO: 14 - RNA, 51 nt, Artificial Sequence
  G3 (CCR5-binding)
gggaggacgaugcgggccuucguuuguuucguccacagacgacucgcccga

SEQ ID NO: 15 - RNA, 86 nt, Artificial Sequence
  C3e2 (CD3E-binding)
ggagacaagaauaaacgcucaaauagaagcagcaucuuccaaaucaguuuguguguccucuauucgacaggaggcucacaacaggc

SEQ ID NO: 16 - RNA, 87 nt, Artificial Sequence
  C3e3 (CD3E-binding)
gggagacaagaauaaacgcucaaaugccuguaguucguagcgauuuaacugcgucagugaggcuucgacaggaggcucacaacaggc

SEQ ID NO: 17 - protein, 113 aa, Artificial Sequence
  36-7 VL
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPRTFGGGTKLEIKR

SEQ ID NO: 18 - protein, 16 aa, Artificial Sequence
  36-7 LC-CDR1
KSSQSLLDSDGKTYLN

SEQ ID NO: 19 - protein, 7 aa, Artificial Sequence
  36-7 LC-CDR2
LVSKLDS

SEQ ID NO: 20 - protein, 9 aa, Artificial Sequence
  36-7 LC-CDR3
WQGTHFPRT

SEQ ID NO: 21 - protein, 112 aa, Artificial Sequence
  36-7 VH
QVQLQQPGAELVKPGASVKLSCKASGYTFTSYWMHWVKQRPGQGLEWIGEIDPSDSYTKYNQKFKGKATLTVDKSSSTAYMQLSSLTSEDSAVYYCARGDYWGQGTTLTVSS

SEQ ID NO: 22 - protein, 5 aa, Artificial Sequence
  36-7 HC-CDR1
SYWMH

SEQ ID NO: 23 - protein, 17 aa, Artificial Sequence
  36-7 HC-CDR2
EIDPSDSYTKYNQKFKG

SEQ ID NO: 24 - protein, 3 aa, Artificial Sequence
  36-7 HC-CDR3
GDY

SEQ ID NO: 25 - protein, 106 aa, Artificial Sequence
  MT-103 VL
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 26 - protein, 10 aa, Artificial Sequence
  MT-103 LC-CDR1
RASSSVSYMN

SEQ ID NO: 27 - protein, 7 aa, Artificial Sequence
  MT-103 LC-CDR2
DTSKVAS

SEQ ID NO: 28 - protein, 9 aa, Artificial Sequence
  MT-103 LC-CDR3
QQWSSNPLT

SEQ ID NO: 29 - protein, 119 aa, Artificial Sequence
  MT-103 VH
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 30 - protein, 5 aa, Artificial Sequence
  MT-103 HC-CDR1
RYTMH

SEQ ID NO: 31 - protein, 17 aa, Artificial Sequence
  MT-103 HC-CDR2
YINPSRGYTNYNQKFKD

SEQ ID NO: 32 - protein, 10 aa, Artificial Sequence
  MT-103 HC-CDR3
YYDDHYCLDY

SEQ ID NO: 33 - DNA, 339 bp, Artificial Sequence
  36-7 VL nucleotide
gacgtggtgatgacccagaccccgctgaccctgagtgtaacgatcggccagcccgccagtatcagctgtaagagcagtcaaagcctgctggacagcgacggcaagacctacctgaactggctgcttcaaaggccgggacagagccccaagaggctcatctacctggtgagcaagctggactcaggcgtgcccgacaggttcaccgggagcgggagtggcaccgacttcaccctgaagatcagcagggtagaggccgaggacctgggtgtgtactactgctggcagggaacccattttcccaggactttcggaggcgggaccaaactggaaatcaagcga

SEQ ID NO: 34 - DNA, 336 bp, Artificial Sequence
  36-7 VH nucleotide
caggtgcagctccaacagccaggtgccgagctggtaaaacctggcgcgtcagtgaagctgagttgtaaagcaagcggctacaccttcaccagctactggatgcactgggttaagcagaggccaggccaaggcctggagtggatcggcgagatcgaccccagcgacagttacaccaagtacaaccagaagttcaagggcaaggcaaccctcacggtcgataagtcaagcagcaccgcgtacatgcaacttagtagcctgaccagcgaggatagcgcggtctactattgcgccagaggcgactactggggccagggcaccactttgaccgtgtctagt

SEQ ID NO: 35 - DNA, 318 bp, Artificial Sequence
  MT-103 VL nucleotide
gacatccagctgacccagagccctgccatcatgagcgccagtcctggcgagaaggtgaccatgacctgtagggcctctagctcagtgagctacatgaactggtatcaacagaaaagcggcacgagccccaagaggtggatctacgacaccagcaaagtggcgtcaggcgtaccttataggtttagcggctcaggatcaggcaccagctacagcctgaccatcagctctatggaggccgaagacgccgccacctattattgccagcagtggtctagcaaccccctgaccttcggagccggcaccaaactggaactgaag

SEQ ID NO: 36 - DNA, 357 bp, Artificial Sequence
  MT-103 VH nucleotide
gacatcaaacttcagcagagcggcgctgagctggctcgaccaggcgcgagcgtgaagatgagctgcaagaccagtggctataccttcaccaggtacaccatgcactgggtgaagcaacgacctggacagggactggagtggatcggctacatcaaccccagcaggggctacaccaactacaaccagaagttcaaggacaaggcgaccctgacgaccgacaagagcagcagcaccgcctacatgcagctgagcagtctcaccagcgaggacagcgcggtgtactactgcgcaaggtactacgacgaccactattgcctggactactggggccagggcaccacgctgacggtgagctct

SEQ ID NO: 37 - protein, 679 aa, Homo sapiens
MISASRAAAARLVGAAASRGPTAARHQDSWNGLSHEAFRLVSRRDYASEAIKGAVVGIDLGTTNSCVAVMEGKQAKVLENAEGARTTPSVVAFTADGERLVGMPAKRQAVTNPNNTFYATKRLIGRRYDDPEVQKDIKNVPFKIVRASNGDAWVEAHGKLYSPSQIGAFVLMKMKETAENYLGHTAKNAVITVPAYFNDSQRQATKDAGQISGLNVLRVINEPTAAALAYGLDKSEDKVIAVYDLGGGTFDISILEIQKGVFEVKSTNGDTFLGGEDFDQALLRHIVKEFKRETGVDLTKDNMALQRVREAAEKAKCELSSSVQTDINLPYLTMDSSGPKHLNMKLTRAQFEGIVTDLIRRTIAPCQKAMQDAEVSKSDIGEVILVGGMTRMPKVQQTVQDLFGRAPSKAVNPDEAVAIGAAIQGGVLAGDVTDVLLLDVTPLSLGIETLGGVFTKLINRNTTIPTKKSQVFSTAADGQTQVEIKVCQGEREMAGDNKLLGQFTLIGIPPAPRGVPQIEVTFDIDANGIVHVSAKDKGTGREQQIVIQSSGGLSKDDIENMVKNAEKYAEEDRRKKERVEAVNMAEGIIHDTETKMEEFKDQLPADECNKLKEEISKMRELLARKDSETGENIRQAASSLQQASLKLFEMAYKKMASEREGSGSSGTGEQKEDQKEEKQ

SEQ ID NO: 38 - protein, 633 aa, Homo sapiens
ASEAIKGAVVGIDLGTTNSCVAVMEGKQAKVLENAEGARTTPSVVAFTADGERLVGMPAKRQAVTNPNNTFYATKRLIGRRYDDPEVQKDIKNVPFKIVRASNGDAWVEAHGKLYSPSQIGAFVLMKMKETAENYLGHTAKNAVITVPAYFNDSQRQATKDAGQISGLNVLRVINEPTAAALAYGLDKSEDKVIAVYDLGGGTFDISILEIQKGVFEVKSTNGDTFLGGEDFDQALLRHIVKEFKRETGVDLTKDNMALQRVREAAEKAKCELSSSVQTDINLPYLTMDSSGPKHLNMKLTRAQFEGIVTDLIRRTIAPCQKAMQDAEVSKSDIGEVILVGGMTRMPKVQQTVQDLFGRAPSKAVNPDEAVAIGAAIQGGVLAGDVTDVLLLDVTPLSLGIETLGGVFTKLINRNTTIPTKKSQVFSTAADGQTQVEIKVCQGEREMAGDNKLLGQFTLIGIPPAPRGVPQIEVTFDIDANGIVHVSAKDKGTGREQQIVIQSSGGLSKDDIENMVKNAEKYAEEDRRKKERVEAVNMAEGIIHDTETKMEEFKDQLPADECNKLKEEISKMRELLARKDSETGENIRQAASSLQQASLKLFEMAYKKMASEREGSGSSGTGEQKEDQKEEKQ

SEQ ID NO: 39 - protein, 466 aa, Homo sapiens
MSTRSVSSSSYRRMFGGPGTASRPSSSRSYVTTSTRTYSLGSALRPSTSRSLYASSPGGVYATRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAENTLQSFRQDVDNASLARLDLERKVESLQEEIAFLKKLHEEEIQELQAQIQEQHVQIDVDVSKPDLTAALRDVRQQYESVAAKNLQEAEEWYKSKFADLSEAANRNNDALRQAKQESTEYRRQVQSLTCEVDALKGTNESLERQMREMEENFAVEAANYQDTIGRLQDEIQNMKEEMARHLREYQDLLNVKMALDIEIATYRKLLEGEESRISLPLPNFSSLNLRETNLDSLPLVDTHSKRTLLIKTVETRDGQVINETSQHHDDLE

SEQ ID NO: 40 - protein, 732 aa, Homo sapiens
MPEETQTQDQPMEEEEVETFAFQAEIAQLMSLIINTFYSNKEIFLRELISNSSDALDKIRYESLTDPSKLDSGKELHINLIPNKQDRTLTIVDTGIGMTKADLINNLGTIAKSGTKAFMEALQAGADISMIGQFGVGFYSAYLVAEKVTVITKHNDDEQYAWESSAGGSFTVRTDTGEPMGRGTKVILHLKEDQTEYLEERRIKEIVKKHSQFIGYPITLFVEKERDKEVSDDEAEEKEDKEEEKEKEEKESEDKPEIEDVGSDEEEEKKDGDKKKKKKIKEKYIDQEELNKTKPIWTRNPDDITNEEYGEFYKSLTNDWEDHLAVKHFSVEGQLEFRALLFVPRRAPFDLFENRKKKNNIKLYVRRVFIMDNCEELIPEYLNFIRGVVDSEDLPLNISREMLQQSKILKVIRKNLVKKCLELFTELAEDKENYKKFYEQFSKNIKLGIHEDSQNRKKLSELLRYYTSASGDEMVSLKDYCTRMKENQKHIYYITGETKDQVANSAFVERLRKHGLEVIYMIEPIDEYCVQQLKEFEGKTLVSVTKEGLELPEDEEEKKKQEEKKTKFENLCKIMKDILEKKVEKVVVSNRLVTSPCCIVTSTYGWTANMERIMKAQALRDNSTMGYMAAKKHLEINPDHSIIETLRQKAEADKNDKSVKDLVILLYETALLSSGFSLEDPQTHANRIYRMIKLGLGIDEDDPTADDTSAAVTEEMPPLEGDDDTSRMEEVD

SEQ ID NO: 41 - protein, 854 aa, Homo sapiens
MPPCSGGDGSTPPGPSLRDRDCPAQSAEYPRDRLDPRPGSPSEASSPPFLRSRAPVNWYQEKAQVFLWHLMVSGSTTLLCLWKQPFHVSAFPVTASLAFRQSQGAGQHLYKDLQPFILLRLLMPEETQTQDQPMEEEEVETFAFQAEIAQLMSLIINTFYSNKEIFLRELISNSSDALDKIRYESLTDPSKLDSGKELHINLIPNKQDRTLTIVDTGIGMTKADLINNLGTIAKSGTKAFMEALQAGADISMIGQFGVGFYSAYLVAEKVTVITKHNDDEQYAWESSAGGSFTVRTDTGEPMGRGTKVILHLKEDQTEYLEERRIKEIVKKHSQFIGYPITLFVEKERDKEVSDDEAEEKEDKEEEKEKEEKESEDKPEIEDVGSDEEEEKKDGDKKKKKKIKEKYIDQEELNKTKPIWTRNPDDITNEEYGEFYKSLTNDWEDHLAVKHFSVEGQLEFRALLFVPRRAPFDLFENRKKKNNIKLYVRRVFIMDNCEELIPEYLNFIRGVVDSEDLPLNISREMLQQSKILKVIRKNLVKKCLELFTELAEDKENYKKFYEQFSKNIKLGIHEDSQNRKKLSELLRYYTSASGDEMVSLKDYCTRMKENQKHIYYITGETKDQVANSAFVERLRKHGLEVIYMIEPIDEYCVQQLKEFEGKTLVSVTKEGLELPEDEEEKKKQEEKKTKFENLCKIMKDILEKKVEKVVVSNRLVTSPCCIVTSTYGWTANMERIMKAQALRDNSTMGYMAAKKHLEINPDHSIIETLRQKAEADKNDKSVKDLVILLYETALLSSGFSLEDPQTHANRIYRMIKLGLGIDEDDPTADDTSAAVTEEMPPLEGDDDTSRMEEVD

SEQ ID NO: 42 - protein, 724 aa, Homo sapiens
MPEEVHHGEEEVETFAFQAEIAQLMSLIINTFYSNKEIFLRELISNASDALDKIRYESLTDPSKLDSGKELKIDIIPNPQERTLTLVDTGIGMTKADLINNLGTIAKSGTKAFMEALQAGADISMIGQFGVGFYSAYLVAEKVVVITKHNDDEQYAWESSAGGSFTVRADHGEPIGRGTKVILHLKEDQTEYLEERRVKEVVKKHSQFIGYPITLYLEKEREKEISDDEAEEEKGEKEEEDKDDEEKPKIEDVGSDEEDDSGKDKKKKTKKIKEKYIDQEELNKTKPIWTRNPDDITQEEYGEFYKSLTNDWEDHLAVKHFSVEGQLEFRALLFIPRRAPFDLFENKKKKNNIKLYVRRVFIMDSCDELIPEYLNFIRGVVDSEDLPLNISREMLQQSKILKVIRKNIVKKCLELFSELAEDKENYKKFYEAFSKNLKLGIHEDSTNRRRLSELLRYHTSQSGDEMTSLSEYVSRMKETQKSIYYITGESKEQVANSAFVERVRKRGFEVVYMTEPIDEYCVQQLKEFDGKSLVSVTKEGLELPEDEEEKKKMEESKAKFENLCKLMKEILDKKVEKVTISNRLVSSPCCIVTSTYGWTANMERIMKAQALRDNSTMGYMMAKKHLEINPDHPIVETLRQKAEADKNDKAVKDLVVLLFETALLSSGFSLEDPQTHSNRIYRMIKLGLGIDEDEVAAEEPNAAVPDEIPPLEGDEDASRMEEVD

SEQ ID NO: 43 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 44 - protein, 801 aa, Homo sapiens
MERLWGLFQRAQQLSPRSSQTVYQRVEGPRKGHLEEEEEDGEEGAETLAHFCPMELRGPEPLGSRPRQPNLIPWAAAGRRAAPYLVLTALLIFTGAFLLGYVAFRGSCQACGDSVLVVSEDVNYEPDLDFHQGRLYWSDLQAMFLQFLGEGRLEDTIRQTSLRERVAGSAGMAALTQDIRAALSRQKLDHVWTDTHYVGLQFPDPAHPNTLHWVDEAGKVGEQLPLEDPDVYCPYSAIGNVTGELVYAHYGRPEDLQDLRARGVDPVGRLLLVRVGVISFAQKVTNAQDFGAQGVLIYPEPADFSQDPPKPSLSSQQAVYGHVHLGTGDPYTPGFPSFNQTQFPPVASSGLPSIPAQPISADIASRLLRKLKGPVAPQEWQGSLLGSPYHLGPGPRLRLVVNNHRTSTPINNIFGCIEGRSEPDHYVVIGAQRDAWGPGAAKSAVGTAILLELVRTFSSMVSNGFRPRRSLLFISWDGGDFGSVGSTEWLEGYLSVLHLKAVVYVSLDNAVLGDDKFHAKTSPLLTSLIESVLKQVDSPNHSGQTLYEQVVFTNPSWDAEVIRPLPMDSSAYSFTAFVGVPAVEFSFMEDDQAYPFLHTKEDTYENLHKVLQGRLPAVAQAVAQLAGQLLIRLSHDRLLPLDFGRYGDVVLRHIGNLNEFSGDLKARGLTLQWVYSARGDYIRAAEKLRQEIYSSEERDERLTRMYNVRIMRVEFYFLSQYVSPADSPFRHIFMGRGDHTLGALLDHLRLLRSNSSGTPGATSSTGFQESRFRRQLALLTWTLQGAANALSGDVWNIDNNF

SEQ ID NO: 45 - protein, 630 aa, Homo sapiens
MAALTQDIRAALSRQKLDHVWTDTHYVGLQFPDPAHPNTLHWVDEAGKVGEQLPLEDPDVYCPYSAIGNVTGELVYAHYGRPEDLQDLRARGVDPVGRLLLVRVGVISFAQKVTNAQDFGAQGVLIYPEPADFSQDPPKPSLSSQQAVYGHVHLGTGDPYTPGFPSFNQTQFPPVASSGLPSIPAQPISADIASRLLRKLKGPVAPQEWQGSLLGSPYHLGPGPRLRLVVNNHRTSTPINNIFGCIEGRSEPDHYVVIGAQRDAWGPGAAKSAVGTAILLELVRTFSSMVSNGFRPRRSLLFISWDGGDFGSVGSTEWLEGYLSVLHLKAVVYVSLDNAVLGDDKFHAKTSPLLTSLIESVLKQVDSPNHSGQTLYEQVVFTNPSWDAEVIRPLPMDSSAYSFTAFVGVPAVEFSFMEDDQAYPFLHTKEDTYENLHKVLQGRLPAVAQAVAQLAGQLLIRLSHDRLLPLDFGRYGDVVLRHIGNLNEFSGDLKARGLTLQWVYSARGDYIRAAEKLRQEIYSSEERDERLTRMYNVRIMRVEFYFLSQYVSPADSPFRHIFMGRGDHTLGALLDHLRLLRSNSSGTPGATSSTGFQESRFRRQLALLTWTLQGAANALSGDVWNIDNNF

SEQ ID NO: 46 - protein, 774 aa, Homo sapiens
MERLWGLFQRAQQLSPRSSQTVYQRVEGPRKGHLEEEEEDGEEGAETLAHFCPMELRGPEPLGSRPRQPNLIPWAAAGRRAAPYLVLTALLIFTGAFLLGYVAFRGSCQACGDSVLVVSEDVNYEPDLDFHQGRLYWSDLQAMFLQFLGEGRLEDTIRQTSLRERVAGSAGMAALTQDIRAALSRQKLDHVWTDTHYVGLQFPDPAHPNTLHWVDEAGKVGEQLPLEDPDVYCPYSAIGNVTGELVYAHYGRPEDLQDLRARGVDPVGRLLLVRVGVISFAQKVTNAQDFGAQGVLIYPEPADFSQDPPKPSLSSQQAVYGHVHLGTGDPYTPGFPSFNQTQKLKGPVAPQEWQGSLLGSPYHLGPGPRLRLVVNNHRTSTPINNIFGCIEGRSEPDHYVVIGAQRDAWGPGAAKSAVGTAILLELVRTFSSMVSNGFRPRRSLLFISWDGGDFGSVGSTEWLEGYLSVLHLKAVVYVSLDNAVLGDDKFHAKTSPLLTSLIESVLKQVDSPNHSGQTLYEQVVFTNPSWDAEVIRPLPMDSSAYSFTAFVGVPAVEFSFMEDDQAYPFLHTKEDTYENLHKVLQGRLPAVAQAVAQLAGQLLIRLSHDRLLPLDFGRYGDVVLRHIGNLNEFSGDLKARGLTLQWVYSARGDYIRAAEKLRQEIYSSEERDERLTRMYNVRIMRVEFYFLSQYVSPADSPFRHIFMGRGDHTLGALLDHLRLLRSNSSGTPGATSSTGFQESRFRRQLALLTWTLQGAANALSGDVWNIDNNF

SEQ ID NO: 47 - protein, 1089 aa, Homo sapiens
MGTSHPAFLVLGCLLTGLSLILCQLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKATSELDLEMEALKTVYKSGETIVVTCAVFNNEVVDLQWTYPGEVKGKGITMLEEIKVPSIKLVYTLTVPEATVKDSGDYECAARQATREVKEMKKVTISVHEKGFIEIKPTFSQLEAVNLHEVKHFVVEVRAYPPPRISWLKNNLTLIENLTEITTDVEKIQEIRYRSKLKLIRAKEEDSGHYTIVAQNEDAVKSYTFELLTQVPSSILDLVDDHHGSTGGQTVRCTAEGTPLPDIEWMICKDIKKCNNETSWTILANNVSNIITEIHSRDRSTVEGRVTFAKVEETIAVRCLAKNLLGAENRELKLVAPTLRSELTVAAAVLVLLVIVIISLIVLVVIWKQKPRYEIRWRVIESISPDGHEYIYVDPMQLPYDSRWEFPRDGLVLGRVLGSGAFGKVVEGTAYGLSRSQPVMKVAVKMLKPTARSSEKQALMSELKIMTHLGPHLNIVNLLGACTKSGPIYIITEYCFYGDLVNYLHKNRDSFLSHHPEKPKKELDIFGLNPADESTRSYVILSFENNGDYMDMKQADTTQYVPMLERKEVSKYSDIQRSLYDRPASYKKKSMLDSEVKNLLSDDNSEGLTLLDLLSFTYQVARGMEFLASKNCVHRDLAARNVLLAQGKIVKICDFGLARDIMHDSNYVSKGSTFLPVKWMAPESIFDNLYTTLSDVWSYGILLWEIFSLGGTPYPGMMVDSTFYNKIKSGYRMAKPDHATSEVYEIMVKCWNSEPEKRPSFYHLSEIVENLLPGQYKKSYEKIHLDFLKSDHPAVARMRVDSDNAYIGVTYKNEEDKLKDWEGGLDEQRLSADSGYIIPLPDIDPVPEEEDLGKRNRHSSQTSEESAIETGSSSSTFIKREDETIEDIDMMDDIGIDSSDLVEDSFL

SEQ ID NO: 48 - protein, 1066 aa, Homo sapiens
QLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKATSELDLEMEALKTVYKSGETIVVTCAVFNNEVVDLQWTYPGEVKGKGITMLEEIKVPSIKLVYTLTVPEATVKDSGDYECAARQATREVKEMKKVTISVHEKGFIEIKPTFSQLEAVNLHEVKHFVVEVRAYPPPRISWLKNNLTLIENLTEITTDVEKIQEIRYRSKLKLIRAKEEDSGHYTIVAQNEDAVKSYTFELLTQVPSSILDLVDDHHGSTGGQTVRCTAEGTPLPDIEWMICKDIKKCNNETSWTILANNVSNIITEIHSRDRSTVEGRVTFAKVEETIAVRCLAKNLLGAENRELKLVAPTLRSELTVAAAVLVLLVIVIISLIVLVVIWKQKPRYEIRWRVIESISPDGHEYIYVDPMQLPYDSRWEFPRDGLVLGRVLGSGAFGKVVEGTAYGLSRSQPVMKVAVKMLKPTARSSEKQALMSELKIMTHLGPHLNIVNLLGACTKSGPIYIITEYCFYGDLVNYLHKNRDSFLSHHPEKPKKELDIFGLNPADESTRSYVILSFENNGDYMDMKQADTTQYVPMLERKEVSKYSDIQRSLYDRPASYKKKSMLDSEVKNLLSDDNSEGLTLLDLLSFTYQVARGMEFLASKNCVHRDLAARNVLLAQGKIVKICDFGLARDIMHDSNYVSKGSTFLPVKWMAPESIFDNLYTTLSDVWSYGILLWEIFSLGGTPYPGMMVDSTFYNKIKSGYRMAKPDHATSEVYEIMVKCWNSEPEKRPSFYHLSEIVENLLPGQYKKSYEKIHLDFLKSDHPAVARMRVDSDNAYIGVTYKNEEDKLKDWEGGLDEQRLSADSGYIIPLPDIDPVPEEEDLGKRNRHSSQTSEESAIETGSSSSTFIKREDETIEDIDMMDDIGIDSSDLVEDSFL

SEQ ID NO: 49 - protein, 505 aa, Homo sapiens
QLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKATSELDLEMEALKTVYKSGETIVVTCAVFNNEVVDLQWTYPGEVKGKGITMLEEIKVPSIKLVYTLTVPEATVKDSGDYECAARQATREVKEMKKVTISVHEKGFIEIKPTFSQLEAVNLHEVKHFVVEVRAYPPPRISWLKNNLTLIENLTEITTDVEKIQEIRYRSKLKLIRAKEEDSGHYTIVAQNEDAVKSYTFELLTQVPSSILDLVDDHHGSTGGQTVRCTAEGTPLPDIEWMICKDIKKCNNETSWTILANNVSNIITEIHSRDRSTVEGRVTFAKVEETIAVRCLAKNLLGAENRELKLVAPTLRSELTVA

SEQ ID NO: 50 - protein, 218 aa, Homo sapiens
MGTSHPAFLVLGCLLTGLSLILCQLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKGTCIISFLL

SEQ ID NO: 51 - protein, 195 aa, Homo sapiens
QLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKGTCIISFLL

SEQ ID NO: 52 - protein, 743 aa, Homo sapiens
MGTSHPAFLVLGCLLTGLSLILCQLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKATSELDLEMEALKTVYKSGETIVVTCAVFNNEVVDLQWTYPGEVKGKGITMLEEIKVPSIKLVYTLTVPEATVKDSGDYECAARQATREVKEMKKVTISVHEKGFIEIKPTFSQLEAVNLHEVKHFVVEVRAYPPPRISWLKNNLTLIENLTEITTDVEKIQEIRYRSKLKLIRAKEEDSGHYTIVAQNEDAVKSYTFELLTQVPSSILDLVDDHHGSTGGQTVRCTAEGTPLPDIEWMICKDIKKCNNETSWTILANNVSNIITEIHSRDRSTVEGRVTFAKVEETIAVRCLAKNLLGAENRELKLVAPTLRSELTVAAAVLVLLVIVIISLIVLVVIWKQKPRYEIRWRVIESISPDGHEYIYVDPMQLPYDSRWEFPRDGLVLGRVLGSGAFGKVVEGTAYGLSRSQPVMKVAVKMLKPTARSSEKQALMSELKIMTHLGPHLNIVNLLGACTKSGPIYIITEYCFYGDLVNYLHKNRDSFLSHHPEKPKKELDIFGLNPADESTRSSGQGCLSSGTLQELSVDLQARGPC

SEQ ID NO: 53 - protein, 720 aa, Homo sapiens
QLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKATSELDLEMEALKTVYKSGETIVVTCAVFNNEVVDLQWTYPGEVKGKGITMLEEIKVPSIKLVYTLTVPEATVKDSGDYECAARQATREVKEMKKVTISVHEKGFIEIKPTFSQLEAVNLHEVKHFVVEVRAYPPPRISWLKNNLTLIENLTEITTDVEKIQEIRYRSKLKLIRAKEEDSGHYTIVAQNEDAVKSYTFELLTQVPSSILDLVDDHHGSTGGQTVRCTAEGTPLPDIEWMICKDIKKCNNETSWTILANNVSNIITEIHSRDRSTVEGRVTFAKVEETIAVRCLAKNLLGAENRELKLVAPTLRSELTVAAAVLVLLVIVIISLIVLVVIWKQKPRYEIRWRVIESISPDGHEYIYVDPMQLPYDSRWEFPRDGLVLGRVLGSGAFGKVVEGTAYGLSRSQPVMKVAVKMLKPTARSSEKQALMSELKIMTHLGPHLNIVNLLGACTKSGPIYIITEYCFYGDLVNYLHKNRDSFLSHHPEKPKKELDIFGLNPADESTRSSGQGCLSSGTLQELSVDLQARGPC

SEQ ID NO: 54 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 55 - protein, 185 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 56 - protein, 104 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 57 - protein, 352 aa, Homo sapiens [sequence omitted]

SEQ ID NO: 58 - RNA, 25 nt, Homo sapiens
agacaagaauaaacgcucaaaguug

SEQ ID NO: 59 - RNA, 22 nt, Homo sapiens
uuuauucacauuuuugaauuga

SEQ ID NO: 60 - protein, 287 aa, Homo sapiens
GLHNHHPIKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSADVPKPSISSNNSKPVEDKDAVAFHCEPEAQNTTYLWWVNGQSLPVSPRLQLSNGNRTLTLFNVTRNDARAYVCGIQNSVSANRSDPVTLDVLYGPDTPIISPPP

SEQ ID NO: 61 - RNA, 26 nt, Artificial Sequence
  Truncated CD3e3
guucguagcgauuuaacugcgucagc

(19) United States
(12) Patent Application Publication
(10) Pub. No.: US 2020/0181264 A1
(43) Pub. Date: Jun. 11, 2020

(54) BISPECIFIC ANTIGEN-BINDING MOLECULE

(71) Applicants: CITY OF HOPE, Duarte, CA (US); APTERNA LIMITED, London (GB)

(72) Inventors: John J. Rossi, Azusa, CA (US); Sarah Yoon, Pasadena, CA (US); Piotr Marek Swiderski, San Dimas, CA (US); Nagy Habib, London (GB); John Ernest Shively, Duarte, CA (US)

(21) Appl. No.: 16/637,705

(22) PCT Filed: Aug. 10, 2018
(86) PCT No.: PCT/US18/46342

§ 371 Date: Feb. 7, 2020

Related U.S. Application Data
(60) Provisional application No. 62/544,220, filed on Aug. 11, 2017.

(51) Int. Cl. C07K 16/28; C07K 16/30; C12N 15/115; A61P 35/00

(52) U.S. Cl. CPC C07K 16/2809; C07K 16/303; C12N 15/115; A61P 35/00; C12N 2310/3513; C12N 2310/16; C07K 2317/31; C07K 2317/24; C07K 2317/565

(57) ABSTRACT

Provided herein relates to antigen-binding molecules, in particular bispecific antigen-binding molecules that bind to a cancer cell antigen and an immune cell surface molecule.

### CROSS-REFERENCES TO RELATED APPLICATIONS

This application claims priority to U.S. Provisional Application No. 62/544,220, filed Aug. 11, 2017, which is hereby incorporated by reference in its entirety and for all purposes.

### REFERENCE TO A “SEQUENCE LISTING,” A TABLE, OR A COMPUTER PROGRAM LISTING APPENDIX SUBMITTED AS AN ASCII FILE

The Sequence Listing written in the file “EFSLIVE-17561671-v1-RICLP7277742_ST25.txt”, created on Aug. 9, 2018, 123,534 bytes, machine format IBM-PC, MS-Windows operating system, is hereby incorporated by reference.

### BACKGROUND

Cancer antigens are often intracellular proteins which are inaccessible to classical therapy with monoclonal antibodies (mAbs). The intracellular protein mortalin (mtHSP70; HSPA9; GRP75) has recently been found to be translocated to the plasma membrane and expressed at the cell surface of cancer cells.

### SUMMARY

Provided herein is a chimeric antigen-binding molecule comprising an aptamer moiety and an antigen-binding polypeptide moiety, which is capable of binding to a cancer cell antigen and an immune cell surface molecule. The antigen-binding molecule described herein is useful to direct immune cell effector function against cancer cell antigen-expressing cells, by bringing the immune cell and cancer cell antigen-expressing cell into close physical proximity. Simultaneous binding of the antigen-binding molecule to the immune cell and the cancer cell antigen-expressing cell can also cause activation/stimulation of the immune cell, enhancing immune cell effector activity, proliferation/survival and recruitment and activation of further immune cells to amplify the response.

In a first aspect, provided herein is an antigen-binding molecule capable of binding to a cancer cell antigen and an immune cell surface molecule, comprising: (i) an aptamer, and (ii) an antigen-binding polypeptide.

In some embodiments the antigen-binding molecule is capable of binding to cells expressing the cancer cell antigen at the cell surface.

In some embodiments the antigen-binding molecule is capable of increasing killing of cells expressing the cancer cell antigen at the cell surface by an immune cell.

In some embodiments the cancer cell antigen is selected from the group consisting of: mortalin, vimentin, HSP90, TfR, PDGFR-a and CEA.

In some embodiments the immune cell surface molecule is selected from the group consisting of: a CD3-TCR complex polypeptide, CD3ε, CD3γ, CD3δ, CD3ζ, CD3η, TCRα, TCRβ, TCRγ, TCRδ, CD27, CD28, CD4, CD8, CCR5, CCR7, CD2, CD7, PD-1, and CTLA4.

In some embodiments the aptamer is a cancer cell antigen-binding aptamer.

In some embodiments the cancer cell antigen-binding aptamer comprises, or consists of, a nucleic acid sequence having at least 80% sequence identity to one of SEQ ID NOs:1 to 13, 58 and 59.

In some embodiments the cancer cell antigen is mortalin.

In some embodiments the cancer cell antigen-binding aptamer comprises, or consists of, a nucleic acid sequence having at least 80% sequence identity to one of SEQ ID NOs:1, 2, 3 and 8.

In some embodiments the antigen-binding polypeptide is an immune cell surface molecule-binding polypeptide.

In some embodiments the immune cell surface molecule-binding polypeptide comprises a CD3ε-binding domain comprising the amino acid sequences i) to vi):

(SEQ ID NO: 26)
i) LC-CDR1: RASSSVSYMN
(SEQ ID NO: 27)
ii) LC-CDR2: DTSKVAS
(SEQ ID NO: 28)
iii) LC-CDR3: QQWSSNPLT
(SEQ ID NO: 30)
iv) HC-CDR1: RYTMH
(SEQ ID NO: 31)
v) HC-CDR2:YINPSRGYTNYNQKFKD
(SEQ ID NO: 32)
vi) HC-CDR3:YYDDHYCLDY;

or a variant thereof in which one or two or three amino acids in one or more of the sequences i) to vi) are replaced with another amino acid.

In some embodiments the immune cell surface molecule-binding polypeptide comprises a CD3ε-binding domain comprising:
a variable light chain (VL) region comprising, or consisting of, an amino acid sequence having at least 70% sequence identity to the amino acid sequence of SEQ ID NO:25; and
a variable heavy chain (VH) region comprising, or consisting of, an amino acid sequence having at least 70% sequence identity to the amino acid sequence of SEQ ID NO:29.

In some embodiments the antigen-binding polypeptide is a cancer cell antigen-binding polypeptide.

In some embodiments the cancer cell antigen-binding polypeptide comprises a mortalin-binding domain comprising the amino acid sequences i) to vi):

(SEQ ID NO: 18)
i) LC-CDR1: KSSQSLLDSDGKTYLN
(SEQ ID NO: 19)
ii) LC-CDR2: LVSKLDS
(SEQ ID NO: 20)
iii) LC-CDR3: WQGTHFPRT
(SEQ ID NO: 22)
iv) HC-CDR1: SYWMH
(SEQ ID NO: 23)
v) HC-CDR2: EIDPSDSYTKYNQKFKG
(SEQ ID NO: 24)
vi) HC-CDR3: GDY;

or a variant thereof in which one or two or three amino acids in one or more of the sequences i) to vi) are replaced with another amino acid.

In some embodiments the immune cell surface molecule-binding polypeptide comprises a mortalin-binding domain comprising:
a variable light chain (VL) region comprising, or consisting of, an amino acid sequence having at least 70% sequence identity to the amino acid sequence of SEQ ID NO:17; and
a variable heavy chain (VH) region comprising, or consisting of, an amino acid sequence having at least 70% sequence identity to the amino acid sequence of SEQ ID NO:21.

In some embodiments the aptamer is an immune cell surface molecule-binding aptamer.

In some embodiments the immune cell surface molecule-binding aptamer comprises, or consists of, a nucleic acid sequence having at least 80% sequence identity to one of SEQ ID NOs:14 to 16 and 61.

In another aspect, provided herein is an antigen-binding molecule capable of binding to a cancer cell antigen and an immune cell surface molecule, comprising: (i) a cancer cell antigen-binding aptamer, and (ii) an immune cell surface molecule-binding polypeptide.

In some embodiments the antigen-binding molecule comprises a mortalin-binding aptamer and a CD3ε-binding polypeptide. In some embodiments the antigen-binding molecule comprises a vimentin-binding aptamer and a CD3ε-binding polypeptide.

In some embodiments the mortalin-binding aptamer comprises, or consists of, a nucleic acid sequence having at least 80% sequence identity to one of SEQ ID NOs:1, 2, 3 and 8.

In some embodiments the CD3ε-binding polypeptide comprises a CD3ε-binding domain comprising:
a variable light chain (VL) region comprising, or consisting of, an amino acid sequence having at least 70% sequence identity to the amino acid sequence of SEQ ID NO:27; and
a variable heavy chain (VH) region comprising, or consisting of, an amino acid sequence having at least 70% sequence identity to the amino acid sequence of SEQ ID NO:31.

In another aspect, provided herein is an antigen-binding molecule capable of binding to a cancer cell antigen and an immune cell surface molecule, comprising: (i) a cancer cell antigen-binding polypeptide, and (ii) an immune cell surface molecule-binding aptamer.

In some embodiments the antigen-binding molecule comprises a CD3ε-binding aptamer and a CEA-binding polypeptide.

In accordance with various aspects described herein, in some embodiments the aptamer and antigen-binding polypeptide are covalently associated with one another.

In another aspect, provided herein is a complex, optionally an in vitro complex, comprising the antigen-binding molecule described herein bound to a cancer cell antigen and/or an immune cell surface molecule.

In another aspect, provided herein is a composition comprising the antigen-binding molecule described herein and at least one pharmaceutically-acceptable carrier, diluent or excipient.

In another aspect the antigen-binding molecule or the composition described herein is provided for use in therapy, or in a method of medical treatment.

In another aspect, the antigen-binding molecule or the composition described herein is provided for use in the treatment or prevention of a cancer.

In another aspect, provided herein is the use of the antigen-binding molecule or the composition described herein, in the manufacture of a medicament for treating or preventing a cancer.

In another aspect, provided herein is a method of treating or preventing a cancer, comprising administering to a subject a therapeutically or prophylactically effective amount of the antigen-binding molecule or the composition described herein.

In another aspect, provided herein is a method of diagnosing or prognosing a cancer in a subject, comprising contacting a sample from the subject with an antigen-binding molecule described herein, and detecting binding of the antigen-binding molecule to a cancer cell antigen and/or an immune cell surface molecule.

In another aspect, provided herein is the antigen-binding molecule or composition for use, the use, or the method described herein, wherein the cancer is selected from the group consisting of: pancreatic cancer, brain cancer, colorectal cancer, liver cancer, breast cancer and gastric cancer.

In another aspect, provided herein is a kit of parts comprising a predetermined quantity of the antigen-binding molecule or the composition described herein.

In another aspect, provided herein is a vimentin-binding aptamer comprising, or consisting of, a nucleic acid sequence having at least 80% sequence identity to SEQ ID NO:58.

In another aspect, provided herein is a TfR-binding aptamer comprising, or consisting of, a nucleic acid sequence having at least 80% sequence identity to SEQ ID NO:59.

### DESCRIPTION

Provided herein relates to antigen-binding molecules capable of binding to a cancer cell antigen and an immune cell surface molecule, comprising: (i) an aptamer, and (ii) an antigen-binding polypeptide. More specifically, provided herein relates to antigen-binding molecules capable of binding to a cancer cell antigen and an immune cell surface molecule. In particular embodiments the cancer cell antigen is mortalin and the immune cell surface molecule is a CD3-TCR complex polypeptide.

Antigen-binding molecules described herein are useful to potentiate cell killing of cancer cells expressing cancer cell antigens by immune cells. Through binding to both the cancer cell antigen and the immune cell surface molecule, the antigen-binding molecule brings immune cells into close proximity to cancer cells, forming a link between the cells, which in turn causes the immune cell to exert e.g. effector activity on the cancer cell. Such constructs provide for specific targeting of e.g. cytotoxic activity of CTLs against cancer cells expressing the cancer cell antigen, and thus represent a targeted therapy expected to have minimal off-target effects.

Cancer Cell Antigens

The antigen-binding molecules described herein bind to a cancer cell antigen.

Herein, a cancer cell antigen is an antigen which is expressed by a cancer cell. A cancer cell antigen may be any peptide/polypeptide, glycoprotein, lipoprotein, glycan, glycolipid, lipid, or fragment thereof. A cancer cell antigen's expression may be associated with a cancer. A cancer cell antigen may be abnormally expressed by a cancer cell (e.g. the cancer cell antigen may be expressed with abnormal localisation), or may be expressed with an abnormal structure by a cancer cell. A cancer cell antigen may be capable of eliciting an immune response.

In some embodiments, the antigen is expressed at the cell surface of the cancer cell (i.e. the cancer cell antigen is a cancer cell surface antigen). In some embodiments, the part of the antigen which is bound by the antigen-binding molecule described herein is displayed on the external surface of the cancer cell (i.e. is extracellular). In some embodiments, the antigen is anchored to the cell membrane, e.g. via a transmembrane domain or other membrane anchor (e.g. a lipid anchor such as a GPI anchor). In some embodiments, the cancer cell antigen is expressed at the cell surface (i.e. is expressed in or at the cell membrane) of a cancerous cell, but is normally expressed inside the cell (i.e. is expressed inside comparable non-cancerous cells).

The cancer cell antigen may be a cancer-associated antigen. In some embodiments the cancer cell antigen is an antigen whose expression is associated with the development, progression or severity of symptoms of a cancer. The cancer-associated antigen may be associated with the cause or pathology of the cancer, or may be expressed abnormally as a consequence of the cancer. In some embodiments, the antigen is an antigen whose expression is upregulated (e.g. at the RNA and/or protein level) by cells of a cancer, e.g. as compared to the level of expression of by comparable non-cancerous cells (e.g. non-cancerous cells derived from the same tissue/cell type).

In some embodiments, the cancer-associated antigen may be preferentially expressed by cancerous cells, and not expressed by comparable non-cancerous cells (e.g. non-cancerous cells derived from the same tissue/cell type). In some embodiments, the cancer-associated antigen may be the product of a mutated oncogene or mutated tumor suppressor gene. In some embodiments, the cancer-associated antigen may be the product of an overexpressed cellular protein, a cancer antigen produced by an oncogenic virus, an oncofetal antigen, or a cell surface glycolipid or glycoprotein.

Cancer cell antigens are reviewed by Zarour H M, DeLeo A, Finn O J, et al. Categories of Tumor Antigens. In: Kufe D W, Pollock R E, Weichselbaum R R, et al., editors. Holland-Frei Cancer Medicine. 6th edition. Hamilton (ON): BC Decker; 2003. Cancer cell antigens include oncofetal antigens: CEA, Immature laminin receptor, TAG-72; oncoviral antigens such as HPV E6 and E7; overexpressed proteins: BING-4, calcium-activated chloride channel 2, cyclin-B1, 9D7, Ep-CAM, EphA3, Her2/neu, telomerase, mesothelin, SAP-1, surviving; cancer-testis antigens: BAGE, CAGE, GAGE, MAGE, SAGE, XAGE, CT9, CT10, NY-ESO-1, PRAME, SSX-2; lineage restricted antigens: MART1, Gp100, tyrosinase, TRP-1/2, MC1R, prostate specific antigen; mutated antigens: 1-catenin, BRCA1/2, CDK4, CML66, Fibronectin, MART-2, p53, Ras, TGF-βRII; post-translationally altered antigens: MUC1, idiotypic antigens: Ig, TCR. Other cancer cell antigens include heat-shock protein 70 (HSP70), heat-shock protein 90 (HSP90), glucose-regulated protein 78 (GRP78), vimentin, nucleolin, feto-acinar pancreatic protein (FAPP), alkaline phosphatase placental-like 2 (ALPPL-2), siglec-5, stress-induced phosphoprotein 1 (STIP1), protein tyrosine kinase 7 (PTK7), and cyclophilin B.

For example, where the cancer cell is a breast cancer cell, the antigen may be one of EpCAM (epithelial cell adhesion molecule), Her2/neu (Human Epidermal growth factor Receptor 2), MUC-1, EGFR (epidermal growth factor receptor), TAG-12 (tumor associated glycoprotein 12), IGF1 R (insulin-like growth factor 1 receptor), TACSTD2 (tumor associated calcium signal transducer 2), CD318, CD340, CD104, or N-cadherin. For example, where the cancer cell is a prostate cancer cell, the antigen may be one of EpCAM, MUC-1, EGFR, PSMA (prostate specific membrane antigen), PSA (prostate specific antigen), TACSTD2, PSCA (prostate stem cell antigen), PCSA (prostate cell surface antigen), CD318, CD104, or N-cadherin. For example, where the cancer cell is a colorectal cancer cell, the antigen may be one of EpCAM, CD66c, CD66e, CEA (carcinoembryonic antigen), TACSTD2, CK20 (cytokeratin 20), CD104, MUC-1, CD318, or N-cadherin. For example, where the cancer cell is a lung cancer cell the antigen may be one or CK18, CK19, CEA, EGFR, TACSTD2, CD318, CD104, or EpCAM. For example, where the cancer cell is a pancreatic cancer cell the antigen may be one of HSP70, mHSP70, vimentin, HSP90, MUC-1, TACSTD2, CEA, CD104, CD318, N-cadherin, or EpCAM1. For example, where the cancer cell is an ovarian cancer cell the antigen may be one of MUC-1, TACSTD2, CD318, CD104, N-cadherin, or EpCAM. For example, where the cancer cell is a bladder cancer cell, the antigen may be one of CD34, CD146, CD62, CD105, CD106, VEGF receptor (vascular endothelial growth factor receptor), MUC-1, TACSTD2, EpCAM, CD318, EGFR, 6B5 or Folate binding receptor. For example, where the cancer cell is a cancer stem cell, the antigen may be one of CD133, CD135, CD117, or CD34. For example, where the cancer cell is a melanoma cancer cell, the antigen may be one of the melanocyte differentiation antigens, oncofetal antigens, cancer specific antigens, SEREX antigens or a combination thereof. Examples of melanocyte differentiation antigens, include but are not limited to tyrosinase, gp75, gp100, MART 1 or TRP-2. Examples of oncofetal antigens include antigens in the MAGE family (MAGE-A1, MAGE-A4), BAGE family, GAGE family or NY-ESO1. Examples of cancer-specific antigens include CDK4 and 13-catenin. Examples of SEREX antigens include D-1 and SSX-2.

In some preferred embodiments the cancer cell antigen is mortalin. Mortalin may also be referred to herein as mitochondrial 70 kDa heat shock protein, mHSP70, HSP70-9, HSPA9, CSA, GRP-75, GRP75, HEL-S-124m, HSPA9B, MOT, MOT2, MTHSP75, PBP74, CRP40, EVPLS, SAAN or SIDBA4. Mortalin belongs to the heat shock protein 70 family of proteins (Reviewed for example in Daugaard et al., FEBS Lett (2007) 581(19)). Mortalin is a heat shock cognate protein, meaning that it is a constitutively-expressed heat shock protein. Mortalin is primarily localised to mitochondria, but is also found in the endoplasmic reticulum, cell membrane and cytoplasmic vesicles. Mortalin has roles in cell proliferation, stress response and maintenance of the mitochondria. The structure and function of mortalin is discussed e.g. in Dores-Silva et al., PLoS One (2015) 10(1):e0117170. Mortalin is thought to be important for biogenesis of mitochondria and functioning of the cell machinery. Mortalin is involved in the importing and folding of nucleus-encoded proteins into the mitochondrial matrix. Mortalin interacts with p53, and in vivo deregulation of mortalin is correlated with age-related diseases and certain cancers. Mortalin displays ATPase activity, and interacts with adenosine nucleotides with high affinity. Mortalin has also been shown to interact with FGF1 (Mizukoshi et al., 1999 Biochem. J. 343 Pt 2 (2): 461-6).

Human mortalin protein is the protein identified by the UniProt Accession No. P38646 (GRP75_HUMAN) shown in SEQ ID NO:37. The N-terminal 46 amino acids of SEQ ID NO:37 constitute a mitochondrial-targeting signal peptide, and so the mature form (i.e. after processing to remove the signal peptide) of human mortalin protein has the amino acid sequence shown in SEQ ID NO:38.

In this specification “mortalin” refers to a mortalin from any species and includes isoforms, fragments, variants or homologues of mortalin from any species. In some embodiments, the mortalin is mammalian mortalin (e.g. cynomolgous, human and/or rodent (e.g. rat and/or murine) mortalin). In some embodiments, the mouse mortalin is the mouse mortalin isoform designated mot-2. Isoforms, fragments, variants or homologues of mortalin may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of immature or mature mortalin from a given species, e.g. human mortalin. Isoforms, fragments, variants or homologues of mortalin may optionally be functional isoforms, fragments, variants or homologues, e.g. having a functional property/activity of the reference mortalin (e.g. full-length human mortalin), as determined by analysis by a suitable assay for the functional property/activity. For example, an isoform, fragment, variant or homologue of mortalin may display one or more of ATPase activity, interaction with p53 or interaction with FGF1.

A fragment of mortalin may be of any length (by number of amino acids), although may optionally be at least 25% of the length of mature mortalin and may have a maximum length of one of 50%, 75%, 80%, 85%, 90%, 91%, 92%, 93%, 94%, 95%, 96%, 97%, 98%, or 99% of the length of mature mortalin. A fragment of mortalin may have a minimum length of 10 amino acids, and a maximum length of one of 15, 20, 25, 30, 40, 50, 100, 150, 200, 250, 300, 350, 400, 450, 500, 550, 600, or 650 amino acids.

In some embodiments, the mortalin has at least 70%, preferably one of 80%, 85%, 90%, 91%, 92%, 93%, 94%, 95%, 96%, 97%, 98%, 99% or 100% amino acid sequence identity to SEQ ID NO:37 or 38.

Mortalin is involved in tumorigenesis and metastasis of a variety of cancers, including colon, brain, and osteosarcoma cancers (Kaul et al. 2007 Exp Gerontol 42(4):263-74; Wadhwa et al. 2006 Int J Cancer 118(12):2973-80), as discussed in Sane et al., Cell Stress Chaperones. 2016 21(2): 313-326. Mortalin has been shown to inactivate p53 during the progression of cancer (Lu et al. 2011 Cell Death Differ. 18(6):1046-56; Sane et al. 2014 Cell Death Dis. 5:e1118), and to enhance cancer proliferation and metastasis through negatively regulating the Raf/MEK/ERK pathway and triggering innate tumor-suppressive mechanisms (Wu et al. 2013 Mol Cell Biol 33(20):4051-67). Mortalin inhibits the pro-apoptotic Bax protein in both p53-dependent and p53-independent fashion (Lu et al. 2011 supra; Yang et al. 2011 J Mol Biol. 2011 414(5):654-66), sequesters and inactivates p53 in the cytoplasm, and localises in the nucleus and inactivates p53-mediated control of centrosome duplication, causing genomic instability. Mortalin nuclear localisation also results in activation of hTERT and hnRNP-K, which promotes carcinogenesis (Ryu et al. 2014 J Biol Chem. 289(36):24832-44). Mortalin also contributes to the epithelial-to-mesenchymal transition, an important step in tumor invasion and metastasis (Chen et al. 2014 Int J Oncol. 44(1):247-55), and also promotes angiogenesis during tumor progression (Yoo et al. 2010 J Gene Med. 12(7):586-95). Mortalin also associates with DJ-1 protein, and this complex is thought to maintain cancer stem cells through the control of oxidative stress (Conte et al. 2009 Dev Biol. 334(1):109-18; Tai-Nagara et al. 2014 Blood 123(1):41-50).

In some embodiments, the cancer cell antigen is vimentin. Vimentin may also be referred to herein as VIM, CTRCT30 or HEL113. Vimentin is a type-Ill intermediate filament (IF) polypeptide which is expressed in by mesenchymal cells, and is known to be involved in maintaining cellular integrity and stress resistance. The structure and function of vimentin is described, for example, in Satelli and Li, Cell Mol Life Sci. (2011) 68(18):3033-46. Vimentin is overexpressed in various epithelial cancers, including prostate cancer, gastrointestinal tumors, tumors of the central nervous system, breast cancer, malignant melanoma, and lung cancer (Satelli and Li, supra).

Human vimentin protein is the protein identified by the UniProt Accession No. P08670 (VIME_HUMAN) shown in SEQ ID NO:39. In this specification “vimentin” refers to a vimentin from any species and includes isoforms, fragments, variants or homologues of vimentin from any species. In some embodiments, the vimentin is mammalian vimentin (e.g. cynomolgous, human and/or rodent (e.g. rat and/or murine) vimentin). Isoforms, fragments, variants or homologues of vimentin may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of vimentin from a given species, e.g. human vimentin. Isoforms, fragments, variants or homologues of vimentin may optionally be functional isoforms, fragments, variants or homologues, e.g. having a functional property/activity of the reference vimentin (e.g. full-length human vimentin), as determined by analysis by a suitable assay for the functional property/activity.

A fragment of vimentin may be of any length (by number of amino acids), although may optionally be at least 25% of the length of vimentin and may have a maximum length of one of 50%, 75%, 80%, 85%, 90%, 91%, 92%, 93%, 94%, 95%, 96%, 97%, 98%, or 99% of the length of vimentin. A fragment of vimentin may have a minimum length of 10 amino acids, and a maximum length of one of 15, 20, 25, 30, 40, 50, 100, 150, 200, 250, 300, 350, 400 or 450 amino acids.

In some embodiments, the vimentin has at least 70%, preferably one of 80%, 85%, 90%, 91%, 92%, 93%, 94%, 95%, 96%, 97%, 98%, 99% or 100% amino acid sequence identity to SEQ ID NO:39.

In some embodiments, the cancer cell antigen is HSP90. HSP90 is a 90 kDa heat shock protein that assists protein folding and stabilisation against heat stress, and facilitates protein degradation. The structure and function of HSP90 is described, for example, in Pearl Biopolymers. (2016) 105(8):594-607. HSP90 is involved in the activation and cellular stabilization of a range of proteins, including oncogenic protein kinases and nuclear steroid hormone receptors. Overexpression of HSP90 in cancer is linked to poor prognosis, e.g. see Bagatell and Whitesell, Mol Cancer Ther August 2004 3; 1021.

In this specification “HSP90” refers to a HSP90 from any species and includes isoforms, fragments, variants or homologues of a HSP90 from any species. In some embodiments, the HSP90 is a mammalian HSP90 (e.g. a cynomolgous, human and/or rodent (e.g. rat and/or murine) HSP90).

In some embodiments, the HSP90 is the protein HSP90A isoform 1 identified by the UniProt Accession No. P07900-1 (HS90A_HUMAN) shown in SEQ ID NO:40. In some embodiments, the HSP90 is the protein HSP90A isoform 2 identified by the UniProt Accession No. P07900-2, shown in SEQ ID NO:41. In some embodiments, the HSP90 is the protein HSP90B identified by the UniProt Accession No. P08238 (HS90B_HUMAN) shown in SEQ ID NO:42.

Isoforms, fragments, variants or homologues of a HSP90 may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of a HSP90 from a given species, e.g. a human HSP90. Isoforms, fragments, variants or homologues of a HSP90 may optionally be functional isoforms, fragments, variants or homologues, e.g. having a functional property/activity of the reference HSP90 (e.g. a full-length human HSP90), as determined by analysis by a suitable assay for the functional property/activity.

A fragment of a HSP90 may be of any length (by number of amino acids), although may optionally be at least 25% of the length of a HSP90 and may have a maximum length of one of 50%, 75%, 80%, 85%, 90%, 91%, 92%, 93%, 94%, 95%, 96%, 97%, 98%, or 99% of the length of a HSP90. A fragment of a HSP90 may have a minimum length of 10 amino acids, and a maximum length of one of 15, 20, 25, 30, 40, 50, 100, 150, 200, 250, 300, 350, 400, 450, 500, 550, 600, 650, 700, 750, 800 or 850 amino acids.

In some embodiments, the HSP90 has at least 70%, preferably one of 80%, 85%, 90%, 91%, 92%, 93%, 94%, 95%, 96%, 97%, 98%, 99% or 100% amino acid sequence identity to SEQ ID NO:40, 41 or 42.

In some embodiments, the cancer cell antigen is transferrin receptor (TfR). As used herein, transferrin receptor (TfR) may refer to transferrin receptor 1 (also be referred to as TFRC, CD71 or TFR1), or transferrin receptor 2 (also referred to as TFR2, HFE3 or TFRC2). The structure and function of TfR is described, for example, in Tortorella and Karagiannis, J Membr Biol. (2014) 247(4):291-307. TfR is a membrane glycoprotein expressed on the cellular surface and mediates cellular uptake of iron from the plasma glycoprotein transferrin. Iron uptake from transferrin involves the binding of transferrin to TfR. The bound transferrin is then internalized through receptor-mediated endocytosis in an endocytic vesicle. The release of transferrin from TfR is induced by a decrease in the pH within the endocytic vescile. TfR is expressed on a broad variety of cells at varying levels. For example, TfR is highly expressed on immature erythroid cells, placental tissue, and rapidly dividing cells, both normal and malignant. Thus, compounds capable of binding to TfR on the surface of TfR-expressing cells and internalizing into the cell would be very useful for targeted delivery of such compounds. Several studies have reported upregulated expression of transferrin receptors on metastatic and drug resistant tumours.

In this specification “TfR” refers to a TfR from any species and includes isoforms, fragments, variants or homologues of a TfR from any species. In some embodiments, the TfR is a mammalian TfR (e.g. a cynomolgous, human and/or rodent (e.g. rat and/or murine) TfR).

In some embodiments, the TfR is the protein TfR1 identified by the UniProt Accession No. P02786 (TFR1_HUMAN) shown in SEQ ID NO:43. In some embodiments, the TfR is the protein TfR isoform a identified by the UniProt Accession No. Q9UP52-1 (TFR2_HUMAN) shown in SEQ ID NO:44. In some embodiments, the TfR is the protein TfR2 isoform 3 identified by the UniProt Accession No. Q9UP52-2 shown in SEQ ID NO:45. In some embodiments, the TfR is the protein TfR2 isoform γ identified by the UniProt Accession No. Q9UP52-3 (TFR2_HUMAN) shown in SEQ ID NO:46.

Isoforms, fragments, variants or homologues of a TfR may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of a TfR from a given species, e.g. a human TfR. Isoforms, fragments, variants or homologues of a TfR may optionally be functional isoforms, fragments, variants or homologues, e.g. having a functional property/activity of the reference TfR (e.g. a full-length human TfR), as determined by analysis by a suitable assay for the functional property/activity. A fragment of a TfR may be of any length (by number of amino acids), although may optionally be at least 25% of the length of a TfR and may have a maximum length of one of 50%, 75%, 80%, 85%, 90%, 91%, 92%, 93%, 94%, 95%, 96%, 97%, 98%, or 99% of the length of a TfR. A fragment of a TfR may have a minimum length of 10 amino acids, and a maximum length of one of 15, 20, 25, 30, 40, 50, 100, 150, 200, 250, 300, 350, 400, 450, 500, 550, 600, 650, 700, 750 or 800 amino acids.

In some embodiments, the TfR has at least 70%, preferably one of 80%, 85%, 90%, 91%, 92%, 93%, 94%, 95%, 96%, 97%, 98%, 99% or 100% amino acid sequence identity to SEQ ID NO:43, 44, 45 or 46.

In some embodiments, the cancer cell antigen is platelet derived growth factor receptor alpha (PDGFR-a). PDGFR-a may also be referred to as PDGFRA, PDGFR-A, PDGFRα, and platelet derived growth factor receptor A. The structure and function of PDGFR-a is described, for example, in Velghe et al., Oncogene (2014) 33(20):2568-76. PDGFR-a is a cell-surface tyrosine kinase receptor implicated in regulating cell proliferation, cellular differentiation, cell growth and development. PDGFR-a is frequently expressed by tumor cells, predominantly by malignant tumor cells. The expression levels of PDGFR-a correlates with tumor growth, invasiveness, drug resistance and poor clinical outcomes. For example, PDGFR-a is highly over-expressed in glioblastoma (GBM). Thus, compounds capable of binding to PDGFRa on the surface of PDGFR-a expressing cells and internalizing into the cell are highly desirable.

In this specification “PDGFR-a” refers to a PDGFR-a from any species and includes isoforms, fragments, variants or homologues of a PDGFR-a from any species. In some embodiments, the PDGFR-a is a mammalian PDGFR-a (e.g. a cynomolgous, human and/or rodent (e.g. rat and/or murine) PDGFR-a).

In some embodiments, the PDGFR-a is the protein identified by the UniProt Accession No. P16234-1 (PGFRA_HUMAN) shown in SEQ ID NO:47, i.e. PDGFR-a isoform 1. The N-terminal 23 amino acids of SEQ ID NO:47 constitute a signal peptide, and so the mature form (i.e. after processing to remove the signal peptide) of human PDGFR-a isoform 1 has the amino acid sequence shown in SEQ ID NO:48. Residues 24 to 528 of SEQ ID NO:47 represent the extracellular domain (ECD) of PDGFR-a isoform 1, corresponding to the amino acid sequence shown SEQ ID NO:49. In some embodiments, the PDGFR-a is the protein identified by the UniProt Accession No. P16234-2 shown in SEQ ID NO:50, i.e. PDGFR-a isoform 2. The N-terminal 23 amino acids of SEQ ID NO:50 constitute a signal peptide, and so the mature form (i.e. after processing to remove the signal peptide) of human PDGFR-a isoform 2 has the amino acid sequence shown in SEQ ID NO:51. In some embodiments, the PDGFR-a is the protein identified by the UniProt Accession No. P16234-3 shown in SEQ ID NO:52, i.e. PDGFR-a isoform 3. The N-terminal 23 amino acids of SEQ ID NO:52 constitute a signal peptide, and so the mature form (i.e. after processing to remove the signal peptide) of human PDGFR-a isoform 3 has the amino acid sequence shown in SEQ ID NO:53. Residues 24 to 528 of SEQ ID NO:52 represent the extracellular domain (ECD) of PDGFR-a isoform 3, corresponding to the amino acid sequence shown SEQ ID NO:49.

Isoforms, fragments, variants or homologues of a PDGFR-a may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of a PDGFR-a from a given species, e.g. a human PDGFR-a. Isoforms, fragments, variants or homologues of a PDGFR-a may optionally be functional isoforms, fragments, variants or homologues, e.g. having a functional property/activity of the reference PDGFR-a (e.g. a full-length human PDGFR-a), as determined by analysis by a suitable assay for the functional property/activity.

A fragment of a PDGFR-a may be of any length (by number of amino acids), although may optionally be at least 25% of the length of a PDGFR-a and may have a maximum length of one of 50%, 75%, 80%, 85%, 90%, 91%, 92%, 93%, 94%, 95%, 96%, 97%, 98%, or 99% of the length of a PDGFR-a. A fragment of a PDGFR-a may have a minimum length of 10 amino acids, and a maximum length of one of 15, 20, 25, 30, 40, 50, 100, 150, 200, 250, 300, 350, 400, 450, 500, 550, 600, 650, 700, 750, 800, 850, 900, 950, 1000 or 1050 amino acids.

In some embodiments, the PDGFR-a has at least 70%, preferably one of 80%, 85%, 90%, 91%, 92%, 93%, 94%, 95%, 96%, 97%, 98%, 99% or 100% amino acid sequence identity to SEQ ID NO:47, 48, 49, 50, 51, 52 or 53.

In some embodiments, the cancer cell antigen is carcinoembryonic antigen (CEA). The structure and function of CEA is described, for example, in Berinstein, J Clin Oncol. (2002) 15; 20(8):2197-207. CEA is a member of the immunoglobulin supergene family, and is thought to play a role in tumorigenesis. CEA protein is processed and presented on major histocompatibility complex (MHC) proteins for multiple alleles, including HLA A2, A3, and A24, and T lymphocytes can recognize the processed epitopes of CEA and can become activated to lyse CEA-expressing cells.

In this specification “CEA” refers to a CEA from any species and includes isoforms, fragments, variants or homologues of a CEA from any species. In some embodiments, the CEA is a mammalian CEA (e.g. a cynomolgous, human and/or rodent (e.g. rat and/or murine) CEA).

In some embodiments, the CEA is the protein identified by the UniProt Accession No. Q13984-1 (Q13984_HUMAN) shown in SEQ ID NO:60.

Isoforms, fragments, variants or homologues of a CEA may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of a CEA from a given species, e.g. a human CEA. Isoforms, fragments, variants or homologues of a CEA may optionally be functional isoforms, fragments, variants or homologues, e.g. having a functional property/activity of the reference CEA (e.g. a full-length human CEA), as determined by analysis by a suitable assay for the functional property/activity.

A fragment of a CEA may be of any length (by number of amino acids), although may optionally be at least 25% of the length of a CEA and may have a maximum length of one of 50%, 75%, 80%, 85%, 90%, 91%, 92%, 93%, 94%, 95%, 96%, 97%, 98%, or 99% of the length of a CEA. A fragment of a CEA may have a minimum length of 10 amino acids, and a maximum length of one of 15, 20, 25, 30, 40, 50, 100, 150, 200, or 250 amino acids.

In some embodiments, the CEA has at least 70%, preferably one of 80%, 85%, 90%, 91%, 92%, 93%, 94%, 95%, 96%, 97%, 98%, 99% or 100% amino acid sequence identity to SEQ ID NO:60.

Immune Cell Surface Molecules

The antigen-binding molecules described herein bind to an immune cell surface molecule. Herein, an immune cell surface molecule is a molecule which is expressed at the cell surface of an immune cell.

An immune cell surface molecule may be any peptide/polypeptide, glycoprotein, lipoprotein, glycan, glycolipid, lipid, or fragment thereof. In some embodiments, an immune cell surface molecule is a peptide/polypeptide containing molecule (including e.g. glycoproteins, lipoproteins etc.) or fragment thereof. In some embodiments, the part of the immune cell surface molecule which is bound by the bispecific antigen-binding molecule described herein is on the external surface of the immune cell (i.e. is extracellular). In some embodiments, the immune cell surface molecule is anchored to the cell membrane, e.g. via a transmembrane domain or other cell membrane anchor (e.g. a lipid anchor such as a GPI anchor).

The immune cell surface molecule may be expressed at the cell surface of any immune cell. In some embodiments, the immune cell may be a cell of hematopoietic origin, e.g. a neutrophil, eosinophil, basophil, dendritic cell, lymphocyte, or monocyte. The lymphocyte may be e.g. a T cell, B cell, natural killer (NK) cell, NKT cell or innate lymphoid cell (ILC), or a precursor thereof (e.g. a thymocyte or pre-B cell). The cell may express one or more CD3 polypeptides (e.g. CD3ε, CD3γ, CD3δ, CD3ζ and/or CD3η), TCR polypeptides (TCRα, TCRβ, TCRγ and/or TCRδ), CD27, CD28, CD4, CD8, CD16, CCR5, CCR7, CD2, CD7, PD-1, and/or CTLA4.

In some embodiments, the immune cell is a T cell. In some embodiments, the T cell is a CD3+ T cell. In some embodiments, the T cell is a CD3+, CD8+ T cell. In some embodiments, the T cell is a CD3+, CD4+ T cell. In some embodiments, the T cell is a cytotoxic T cell (e.g. a cytotoxic T lymphocyte (CTL)), a T helper cell (e.g. a Th1, Th2, Th9, Th17, Th22 or Tfh cell), a regulatory T cell (Treg), a central memory cell (Tcm), or an effector memory cell (Tem).

In some embodiments, the immune cell is an NK cell. In some embodiments, the NK cell is a CD16+NK cell.

In some embodiments, the immune cell surface molecule is selected from one or more CD3 polypeptides (e.g. CD3ε, CD3γ, CD3δ, CD3ζ or CD3η), TCR polypeptides (TCRα, TCRβ, TCRγ and TCRδ), CD27, CD28, CD4, CD8, CCR5, CCR7, CD2, CD7, PD-1, and CTLA4.

CD3 is a complex of polypeptides expressed at the cell surface of T lymphocytes. In mammals, the CD3 complex contains a CD3γ chain, a CD3δ chain and two CD3ε chains. These chains associate with the TCR polypeptides (TCRα and TCRβ) and CD3ζ/CD3η chains to form the CD3-TCR complex, which generates the activation signal in T lymphocytes.

In some embodiments, the immune cell surface molecule is a molecule expressed at the cell surface of a T cell. In some embodiments, the immune cell surface molecule is a polypeptide of the CD3-TCR complex. In some embodiments, the immune cell surface molecule is a CD3 polypeptide (e.g. CD3ε, CD3γ, CD3δ, CD3ζ or CD3η), a complex containing a CD3 polypeptide, a TCR polypeptide (TCRα, TCRβ, TCRγ or TCRδ) or a complex containing a TCR polypeptide.

In some embodiments, the immune cell surface molecule is CD3ε. CD3ε may also be referred to as CD3E, IMD18, T3E or TCRE. Human CD3ε is the protein identified by the UniProt Accession No. P07766 (CD3E_HUMAN) shown in SEQ ID NO:54. Residues 1 to 22 are constitute a signal peptide, and so the amino acid sequence of the mature protein corresponds to residues 23 to 207 of SEQ ID NO:54, shown in SEQ ID NO:55. Residues 23 to 126 of SEQ ID NO:54 form the extracellular domain of CD3ε, shown in SEQ ID NO:56.

In this specification “CD3ε” refers to CD3ε from any species and includes isoforms, fragments, variants or homologues of CD3ε from any species. In some embodiments, the CD3ε is mammalian CD3ε (e.g. cynomolgous, human and/or rodent (e.g. rat and/or murine) CD3ε). Isoforms, fragments, variants or homologues of vimentin may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of CD3ε from a given species, e.g. human CD3ε. Isoforms, fragments, variants or homologues of CD3ε may optionally be functional isoforms, fragments, variants or homologues, e.g. having a functional property/activity of the reference CD3ε (e.g. full-length human CD3ε), as determined by analysis by a suitable assay for the functional property/activity.

A fragment of CD3ε may be of any length (by number of amino acids), although may optionally be at least 25% of the length of CD3ε and may have a maximum length of one of 50%, 75%, 80%, 85%, 90%, 91%, 92%, 93%, 94%, 95%, 96%, 97%, 98%, or 99% of the length of CD3ε. A fragment of CD3ε may have a minimum length of 10 amino acids, and a maximum length of one of 15, 20, 25, 30, 40, 50, 100, 150 or 200 amino acids.

In some embodiments, the CD3ε has at least 70%, preferably one of 80%, 85%, 90%, 91%, 92%, 93%, 94%, 95%, 96%, 97%, 98%, 99% or 100% amino acid sequence identity to SEQ ID NO:54, 55 or 56.

In some embodiments, the immune cell surface molecule is CCR5. In some embodiments, the immune cell surface molecule is CCR5. CCR5 may also be referred to as CCR5, CC-CKR-5, CCCKR5, CCR-5, CD195, CKR-5, CKR5, CMKBR5, IDDM22 or C-C motif chemokine receptor 5. Human CCR5 is the protein identified by the UniProt Accession No. P51681 (CCR5_HUMAN) shown in SEQ ID NO:57.

In this specification “CCR5” refers to CCR5 from any species and includes isoforms, fragments, variants or homologues of CCR5 from any species. In some embodiments, the CCR5 is mammalian CCR5 (e.g. cynomolgous, human and/or rodent (e.g. rat and/or murine) CCR5). Isoforms, fragments, variants or homologues of vimentin may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of CCR5 from a given species, e.g. human CCR5. Isoforms, fragments, variants or homologues of CCR5 may optionally be functional isoforms, fragments, variants or homologues, e.g. having a functional property/activity of the reference CCR5 (e.g. full-length human CCR5), as determined by analysis by a suitable assay for the functional property/activity.

A fragment of CCR5 may be of any length (by number of amino acids), although may optionally be at least 25% of the length of CCR5 and may have a maximum length of one of 50%, 75%, 80%, 85%, 90%, 91%, 92%, 93%, 94%, 95%, 96%, 97%, 98%, or 99% of the length of CCR5. A fragment of CCR5 may have a minimum length of 10 amino acids, and a maximum length of one of 15, 20, 25, 30, 40, 50, 100, 150, 200, 250, 300 or 350 amino acids.

In some embodiments, the CCR5 has at least 70%, preferably one of 80%, 85%, 90%, 91%, 92%, 93%, 94%, 95%, 96%, 97%, 98%, 99% or 100% amino acid sequence identity to SEQ ID NO:57.

Aptamers

Antigen-binding molecules described herein comprise an aptamer. As used herein, an “aptamer” refers to a nucleic acid which binds (e.g. with high affinity and specificity) to a target molecule e.g. a peptide/polypeptide, glycoprotein, lipoprotein, glycan, glycolipid, lipid, or small molecule. Aptamer structure and properties are reviewed, for example, in Zhou and Rossi Nat Rev Drug Discov. 2017 16(3):181-202.

“Nucleic acid” refers to deoxyribonucleotides or ribonucleotides and polymers thereof in either single-, double- or multiple-stranded form, or complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof.

Examples of polynucleotides contemplated herein include single and double stranded DNA, single and double stranded RNA (including siRNA), and hybrid molecules having mixtures of single and double stranded DNA and RNA. Nucleic acids can be linear or branched. For example, nucleic acids can be a linear chain of nucleotides or the nucleic acids can be branched, e.g., such that the nucleic acids comprise one or more arms or branches of nucleotides. The branched nucleic acids may be repetitively branched to form higher ordered structures such as dendrimers and the like.

Nucleic acids, including nucleic acids with a phosphothioate backbone can include one or more reactive moieties. As used herein, the term reactive moiety includes any group capable of reacting with another molecule, e.g., a nucleic acid or polypeptide through covalent, noncovalent or other interactions. By way of example, the nucleic acid can include an amino acid reactive moiety that reacts with an amino acid on a protein or polypeptide through a covalent, non-covalent or other interaction.

“Nucleic acid” as used herein also encompass nucleic acids containing known nucleotide/nucleoside analogs or modified backbone residues or linkages, which are synthetic, naturally occurring, and non-naturally occurring. The nucleic acids containing nucleotide/nucleoside analogs or modified backbone residues or linkages may have similar binding properties as a reference nucleic acid (lacking the analog/modification), and may be metabolized in a manner similar to the reference nucleic acid. Examples of such analogs include, without limitation, phosphodiester derivatives including, e.g., phosphoramidate, phosphorodiamidate, phosphorothioate (also known as phosphothioate), phosphorodithioate, phosphonocarboxylic acids, phosphonocarboxylates, phosphonoacetic acid, phosphonoformicacid, methyl phosphonate, boron phosphonate, or O-methylphosphoroamidite linkages (see Eckstein, Oligonucleotides and Analogues: A Practical Approach, Oxford University Press); and peptide nucleic acid backbones and linkages. Other analog nucleic acids include those with positive charge backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

An aptamer may be referred to as an oligonucleotide-based target-binding moiety. An “oligonucleotide” as used herein refers to a molecule comprising a chain of two or more nucleotides, and generally comprising fewer than 100 nucleotides. An oligonucleotide may comprise fewer than 100, fewer than 90, fewer than 80, fewer than 70, fewer than 60 or fewer than 50 nucleotides.

Aptamers may comprise, or consist of, RNA or DNA, and may be single-stranded or double-stranded. Aptamers may have secondary or tertiary structure and, thus, may be able to fold into diverse and intricate molecular structures. Almost every aptamer identified to date is a non-naturally occurring molecule.

Aptamers may comprise chemically modified nucleotides or nucleosides, for example in which the sugar and/or phosphate and/or base is chemically modified. Such modifications may improve the stability of the aptamer or make the aptamer more resistant to degradation. The aptamers of the present disclosure may include chemical modifications as described herein such as a chemical substitution at a sugar position, a phosphate position, and/or a base position of the nucleic acid including, for example, incorporation of a modified nucleotide, incorporation of a capping moiety (e.g. 3′ capping), conjugation to a high molecular weight, non-immunogenic compound (e.g. polyethylene glycol (PEG)), conjugation to a lipophilic compound, substitutions in the phosphate backbone. Base modifications may include 5-position pyrimidine modifications, modifications at exocyclic amines, substitution of 4-thiouridine, substitution of 5-bromo- or 5-iodo-uracil, backbone modifications. Sugar modifications may include 2′-amine nucleotides (2′-NH₂), 2′-fluoro nucleotides (2′-F), and 2′-O-methyl (2′-OMe) nucleotides. A wide range of nucleotide, nucleoside, base and phosphate modifications are known to those or ordinary skill in the art, e.g. as described in Eaton et al., Bioorganic & Medicinal Chemistry, Vol. 5, No. 6, pp 1087-1096, 1997.

Aptamers may be synthesised by methods which are well known to the skilled person. For example, aptamers may be chemically synthesised, e.g. on a solid support. Solid phase synthesis may use phosphoramidite chemistry. Briefly, a solid supported nucleotide is detritylated, then coupled with a suitably activated nucleoside phosphoramidite to form a phosphite triester linkage. Capping may then occur, followed by oxidation of the phosphite triester with an oxidant, typically iodine. The cycle may then be repeated to assemble the aptamer (e.g., see Sinha, N. D.; Biernat, J.; McManus, J.; Koster, H. Nucleic Acids Res. 1984, 12, 4539; and Beaucage, S. L.; Lyer, R. P. (1992). Tetrahedron 48 (12): 2223).

Aptamers can be selected in vitro from very large libraries of randomized sequences by the process of systemic evolution of ligands by exponential enrichment (SELEX as described in Ellington A D, Szostak J W (1990) In vitro selection of RNA molecules that bind specific ligands. Nature 346:818-822; Tuerk C, Gold L (1990) Systematic evolution of ligands by exponential enrichment: RNA ligands to bacteriophage T4 DNA polymerase. Science 249:505-510) or by developing SOMAmers (slow off-rate modified aptamers) (Gold L et al. (2010) Aptamer-based multiplexed proteomic technology for biomarker discovery. PLoS ONE 5(12):el 5004). Applying the SELEX and the SOMAmer technology includes for instance adding functional groups that mimic amino acid side chains to expand the aptamer's chemical diversity. As a result high affinity aptamers for a protein may be enriched and identified. Aptamers may exhibit many desirable properties for targeted drug delivery, such as ease of selection and synthesis, high binding affinity and specificity, low immunogenicity, and versatile synthetic accessibility. Anticancer agents (e.g. chemotherapy drugs, toxins, and siRNAs) may be successfully delivered to cancer cells in vitro using aptamers.

Aptamers can be thought of as the nucleic acid equivalent of monoclonal antibodies and often have Kd's in the nM or pM range, e.g. less than one of 500 nM, 100 nM, 50 nM, 10 nM, 1 nM, 500 pM, 100 pM. Aptamers are normally mono-specific, i.e. having high affinity and specificity for a single target molecule. As with monoclonal antibodies, they may be useful in virtually any situation in which target binding is required, including use in therapeutic and diagnostic applications, in vitro or in vivo. In vitro diagnostic applications may include use in detecting the presence or absence of a target molecule.

An aptamer of the antigen-binding molecule described herein may be capable of binding to a cancer cell antigen, e.g. a cancer cell antigen as described herein. In some embodiments, the aptamer is capable of binding to a cancer cell antigen selected from the group consisting of: mortalin, vimentin, HSP90, TfR, PDGFR-a and CEA.

In some embodiments, the aptamer is capable of binding to mortalin. Mortalin-binding aptamers such as P19, tP19 and P1 are described e.g. in WO 2013/154735 A1, which is specifically incorporated herein by reference. In some embodiments the aptamer capable of binding to mortalin comprises, or consists of, one of SEQ ID NOs:1, 2, 3 or 8, or a nucleic acid sequence having at least 80% sequence identity to one of SEQ ID NOs:1, 2, 3 or 8.

In some embodiments, the aptamer is capable of binding to vimentin. Vimentin-binding aptamers such as P15 are described e.g. in WO 2013/154735 A1, which is specifically incorporated herein by reference. In some embodiments the aptamer capable of binding to vimentin comprises, or consists of, SEQ ID NO:4 or 58, or a nucleic acid sequence having at least 80% sequence identity to SEQ ID NO:4 or 58.

In some embodiments, the aptamer is capable of binding to HSP90. HSP90-binding aptamers such as P11, P7 and P6 are described e.g. in WO 2013/154735 A1, which is specifically incorporated herein by reference. In some embodiments the aptamer capable of binding to HSP90 comprises, or consists of, one of SEQ ID NOs:5, 6, 7 and 8, or a nucleic acid sequence having at least 80% sequence identity to one of SEQ ID NOs:5, 6, 7 and 8.

In some embodiments, the aptamer is capable of binding to TfR. TfR-binding aptamers such as TR14 and TR18 are described e.g. in WO 2016/061386 A1, which is specifically incorporated herein by reference. In some embodiments the aptamer capable of binding to TfR comprises, or consists of, one of SEQ ID NOs:9, 10, 11 and 59, or a nucleic acid sequence having at least 80% sequence identity to one of SEQ ID NOs:9, 10, 11 and 59.

In some embodiments, the aptamer is capable of binding to PDGFR-a. PDGFR-a-binding aptamers such as PDR3 and PDR9 are described e.g. in WO 2016/061401 A1, which is specifically incorporated herein by reference. In some embodiments the aptamer capable of binding to PDGFR-a comprises, or consists of, SEQ ID NO:12 or 13, or a nucleic acid sequence having at least 80% sequence identity to SEQ ID NO:12 or 13.

An aptamer of the antigen-binding molecule described herein may be capable of binding to an immune cell surface molecule, e.g. an immune cell surface molecule as described herein. In some embodiments, the aptamer is capable of binding to an immune cell surface molecule selected from: one or more CD3 polypeptides (e.g. CD3ε, CD3γ, CD3δ, CD3ζ and/or CD3η), TCR polypeptides (TCRα, TCRβ, TCRγ and/or TCRδ), CD27, CD28, CD4, CD8, CD16, CCR5, CCR7, CD2, CD7, PD-1, and/or CTLA4. In some embodiments, the aptamer is capable of binding to an immune cell surface molecule selected from CD3ε and CCR5.

In some embodiments, the aptamer is capable of binding to CD4. CD4-binding aptamers are described in Zhou et al., Langmuir (2012) 28(34):12544-12549, Zhang et al., American Journal of Clinical Pathology, (2010) 134(4) and Wheeler et al., J Clin Invest. (2011) 121(6):2401-2412, each specifically incorporated herein by reference.

In some embodiments, the aptamer is capable of binding to CD7. CD7-binding aptamers are described in WO 2014/147559 A1, specifically incorporated herein by reference.

In some embodiments, the aptamer is capable of binding to CD8. CD8-binding aptamers are described in Wang et al., J Allergy Clin Immunol. (2013) 132(3):713-722 and Oelkrug et al., Journal of Cellular and Molecular Medicine (2015) 19:11-20, both of which are specifically incorporated herein by reference.

In some embodiments, the aptamer is capable of binding to PD-1. PD-1-binding aptamers are described in, Prodeus et al. Molecular Therapy Nucleic Acids (2015) 4:e237, Ti-Hsuan Ku, Sensors (2015) 15, 16281-16313, and WO2016/019270 A1, each of which are specifically incorporated herein by reference.

In some embodiments, the aptamer is capable of binding to CTLA4. CTLA4-binding aptamers are described in Herrmann et al., J Clin Invest. (2014)124(7):2977-2987, Gilboa et al., Clin Cancer Res; 19(5); 1054-62, and Santulli-Marotto et al., Cancer Res. (2003) 63(21):7483-9, each specifically incorporated herein by reference.

In some embodiments, the aptamer is capable of binding to CD3ε. In some embodiments the aptamer capable of binding to CD3ε comprises, or consists of, SEQ ID NO:15, 16 or 61, or a nucleic acid sequence having at least 80% sequence identity to SEQ ID NO:15, 16 or 61.

In some embodiments, the aptamer is capable of binding to CCR5. CCR5-binding aptamers are described e.g. in Zhou et al., Chemistry & Biology (2015) 22: 379-390 and US 20160053265 A1, which are specifically incorporated herein by reference. In some embodiments the aptamer capable of binding to CCR5 comprises, or consists of, SEQ ID NO:14, or a nucleic acid sequence having at least 80% sequence identity to SEQ ID NO:14.

Where a nucleic acid sequence has at least 80% (i.e. 80% or more) sequence identity to a reference sequence, the nucleic acid sequence may have e.g. 80%, 85%, 90%, 91%, 92%, 93%, 94%, 95%, 96%, 97%, 98%, 99% or 100% sequence identity to the reference sequence. In embodiments, nucleic acid sequence may have 80%, 85%, 90%, 91%, 92%, 93%, 94%, 95%, 96%, 97%, 98%, 99% or 100% sequence identity to the entire sequence of the reference sequence or to continuous portions (e.g., a portion of 10, 20, 30, 40, 50, 60, 70, 80, 90 continuous nucleotides) of the reference sequence. In some embodiments, the nucleic acid sequence has at least 80% (i.e. 80% or more) sequence identity to a nucleic acid that hybridizes to a given sequence.

In some embodiments, in the aptamer may comprise one or more additional nucleotides in addition to the target-binding sequence (i.e. the cancer cell antigen-binding or immune cell surface molecule-binding sequence). For example, an aptamer of the antigen-binding molecule described herein may comprise 1, 2, 3, 4, 5, 6, 7, 8, 9, 10, 11, 12, 13, 14, 15, 16, 17, 18, 19 or 20 nucleotides at one or both end(s) of the target-binding sequence.

In some embodiments, the aptamer of the antigen-binding molecule described herein may be, or may comprise, ribonucleic acid (RNA). In some embodiments, the aptamers of the antigen-binding molecule described herein may be, or may comprise, deoxyribonucleic acid (DNA). The aptamer may be single-stranded.

Aptamers of the antigen-binding molecule described herein may comprise one or more chemically-modified bases or nucleotides. In some embodiments, one or more nucleotides are chemically modified at the 2′ position of ribose. Such modifications may include O-methyl modification (2′-OMe), fluoride modification (2′-F) or amine modification (2-NH₂). In some embodiments, the aptamers may comprise one or more 2′-fluoro modified pyrimidine. In some embodiments, the aptamers may comprise one or more 2′-O-methylated purine. In some embodiments, the aptamers may comprise one or more 2′-O-methylated purine. In some embodiments, each base of a given type (e.g. A, T, C, G, U) may contain the same chemical modification. In some embodiments the ribose of certain nucleotides (e.g. A, T, C, G, U) may be independently modified.

Aptamers of the antigen-binding molecule described herein may be less than 100 (99 or fewer) nucleotides in length. The length calculation may optionally exclude nucleotides or carbon moieties of any spacer, linker or sticky bridge which forms part of the antigen-binding molecule. The length calculation may also optionally exclude any compound moiety conjugated to the aptamer (e.g. any nucleic acid moiety such as siRNA, saRNA, miRNA etc.).

Where the nucleic sequence is less than 100 (99 or fewer) nucleotides in length, the sequence of the aptamer may be one of 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 nucleotides in length. In some embodiments, the sequence is fewer than 90 nucleotides in length. In some embodiments, the sequence is fewer than 80 nucleotides in length. In some embodiments, the sequence is fewer than 70 nucleotides in length. In some embodiments, the sequence is fewer than 60 nucleotides in length. In some embodiments, the sequence is fewer than 50 nucleotides in length. In some embodiments, the sequence is fewer than 40 nucleotides in length.

In some embodiments, the sequence is between 20 and 99 nucleotides in length. In some embodiments, the sequence is between 25 and 99 nucleotides in length. In some embodiments, the sequence is between 30 and 99 nucleotides in length. In some embodiments, the sequence is between 35 and 99 nucleotides in length. In some embodiments, the sequence is between 40 and 99 nucleotides in length. In some embodiments, the sequence is between 45 and 99 nucleotides in length. In some embodiments, the sequence is between 50 and 99 nucleotides in length. In some embodiments, the sequence is between 55 and 99 nucleotides in length. In some embodiments, the sequence is between 60 and 99 nucleotides in length. In some embodiments, the sequence is between 65 and 99 nucleotides in length. In some embodiments, the sequence is between 70 and 99 nucleotides in length. In some embodiments, the sequence is between 75 and 99 nucleotides in length. In some embodiments, the sequence is between 80 and 99 nucleotides in length. In some embodiments, the sequence is between 85 and 99 nucleotides in length.

Methods for synthesising DNA and RNA aptamers are well known in the art, and are described, for example, in Roy and Caruthers, Molecules (2013) 18:14268-14284, which is hereby incorporated by reference in its entirety. Suitable methods include synthesis according to the phosphoramidite method.

Antigen-Binding Polypeptides

Antigen-binding molecules described herein comprise an antigen-binding polypeptide.

As used herein, a “polypeptide” refers to a molecule comprising a chain of two or more peptides linked by peptide bonds; a “peptide” refers to a chain of two or more amino acid monomers linked by peptide bonds. In some embodiments, a polypeptide may comprise at least 20, 30, 40 or 50 amino acids.

An “antigen-binding polypeptide” refers to a polypeptide which is capable of binding to a target molecule. Examples of antigen-binding polypeptides include antibodies and antigen-binding antibody fragments. An “antibody” is used herein in the broadest sense, and encompasses monoclonal antibodies, polyclonal antibodies, monospecific and multispecific antibodies (e.g., bispecific antibodies), and antibody fragments, as long as they display binding to the relevant target molecule.

The antigen-binding polypeptide described herein preferably displays specific binding to the relevant target e.g., the cancer cell surface antigen, or the immune cell surface antigen). An antigen-binding polypeptide that specifically binds to a target molecule preferably binds the target with greater affinity, and/or with greater duration than it binds to other, non-target molecules.

In some embodiments, the antigen-binding polypeptide displays specific binding for the target antigen. As used herein, “specific binding” refers to binding which is selective for the antigen, and which can be discriminated from non-specific binding to non-target antigen.

An antigen-binding polypeptide of the antigen-binding molecule described herein may be capable of binding to an immune cell surface molecule, e.g. an immune cell surface molecule as described herein. In some embodiments, the antigen-binding polypeptide is capable of binding to an immune cell surface molecule selected from: one or more CD3 polypeptides (e.g. CD3ε, CD3γ, CD3δ, CD3ζ and/or CD3η), TCR polypeptides (TCRα, TCRβ, TCRγ and/or TCRδ), CD27, CD28, CD4, CD8, CD16, CCR5, CCR7, CD2, CD7, PD-1, and/or CTLA4.

An antigen-binding polypeptide of the antigen-binding molecule described herein may be capable of binding to a cancer cell antigen, e.g. a cancer cell antigen as described herein. In some embodiments, the antigen-binding polypeptide is capable of binding to a cancer cell antigen selected from the group consisting of: mortalin, vimentin, HSP90, TfR, PDGFR-a and CEA.

The ability of a given polypeptide to bind specifically to a given molecule can be determined by analysis according to methods known in the art, such as by ELISA, Surface Plasmon Resonance (SPR; see e.g. Hearty et al., Methods Mol Biol (2012) 907:411-442), Bio-Layer Interferometry (see e.g. Lad et al., (2015) J Biomol Screen 20(4): 498-507), flow cytometry, or by a radiolabeled antigen binding assay (RIA) enzyme-linked immunosorbent assay. Through such analysis binding to a given molecule can be measured and quantified. In some embodiments, the binding may be the response detected in a given assay.

In some embodiments, the extent of binding of the antigen-binding polypeptide to an non-target molecule is less than about 10% of the binding of the antibody to the target molecule as measured, e.g. by ELISA, SPR, Bio-Layer Interferometry or by RIA. Alternatively, binding specificity may be reflected in terms of binding affinity where the antigen-binding polypeptide binds with a dissociation constant (KD) that is at least 0.1 order of magnitude (i.e. 0.1×10ⁿ, where n is an integer representing the order of magnitude) greater than the KD of the antigen-binding polypeptide towards a non-target molecule. This may optionally be one of at least 0.2, 0.3, 0.4, 0.5, 0.6, 0.7, 0.8, 0.9, 1.0, 1.5, or 2.0.

In certain embodiments, the antigen-binding polypeptide binds to the target molecule with a KD of ≤10 μM, ≤1 μM, ≤100 nM, ≤10 nM, ≤1 nM, ≤0.1 nM, ≤0.01 nM, or ≤0.001 nM as determined by analysis according to SPR, Bio-Layer Interferometry or by RIA.

In some embodiments, the antigen-binding polypeptide binds to the same or an overlapping epitope of the target molecule as an antibody which is capable of binding to the target molecule (i.e. the cancer cell surface antigen, or the immune cell surface antigen). In some embodiments, the antigen-binding polypeptide displays competitive binding with an antibody which is capable of binding to the target molecule. Whether a given antigen-binding polypeptide displays such competitive binding can be determined by various methods known to the skilled person, including competition ELISA.

In some embodiments, the antigen-binding polypeptide comprises the complementarity-determining regions (CDRs) of an antibody which is capable of binding to the target molecule (i.e. the cancer cell surface antigen, or the immune cell surface antigen). Antibodies generally comprise six CDRs; three in the light chain variable region (VL): LC-CDR1, LC-CDR2, LC-CDR3, and three in the heavy chain variable region (VH): HC-CDR1, HC-CDR2 and HC-CDR3. The six CDRs together define the paratope of the antibody, which is the part of the antibody which binds to the target molecule. There are several different conventions for defining antibody CDRs, such as those described in Kabat et al., Sequences of Proteins of Immunological Interest, 5th Ed. Public Health Service, National Institutes of Health, Bethesda, Md. (1991), Chothia et al., J. Mol. Biol. 196:901-917 (1987), and VBASE2, as described in Retter et al., Nucl. Acids Res. (2005) 33 (suppl 1): D671-D674. Unless otherwise specified, CDRs of the antigen-binding polypeptides described herein are defined according to Kabat et al., Sequences of Proteins of Immunological Interest, 5th Ed. Public Health Service, National Institutes of Health, Bethesda, Md. (1991).

The antigen-binding polypeptide may be designed and prepared using the sequences of monoclonal antibodies (mAbs) capable of binding to the cancer cell antigens, and mAbs capable binding to the immune cell surface molecules. Antigen-binding regions of antibodies, such as single chain variable fragment (scFv), Fab and Fab₂ fragments may also be used/provided. An ‘antigen-binding region’ is any fragment of an antibody which is capable of binding to the target for which the given antibody is specific.

In some embodiments, the antigen-binding molecule described herein comprises an antigen-binding polypeptide comprising the CDRs of an antibody capable of binding to an immune cell surface molecule as described herein. In some embodiments the antigen-binding polypeptide comprises the CDRs of an antibody capable of binding to a cancer cell antigen as described herein. In some embodiments, one or more of the CDRs of the antigen-binding polypeptide may be a variant of the reference CDR(s), e.g. comprising 1, 2 or 3 substitutions with respect to the amino acid sequence of the reference CDR(s).

In some embodiments, the antigen-binding molecule described herein comprises an antigen-binding polypeptide comprising the VH and/or VL domains of antibody capable of binding to an immune cell surface molecule as described herein. In some embodiments, the antigen-binding polypeptide comprises the VH and/or VL domains of an antibody capable of binding to a cancer cell antigen as described herein.

In some embodiments, the VL and/or VH domain of the antigen-binding polypeptide may be a variant of the reference VL/VH domain, e.g. comprising 1, 2, 3, 4, 5, 6, 7, 8, 9 or 10 substitutions with respect to the amino acid sequence of the reference domain(s).

The VL and VH region of an antigen-binding region of an antibody together constitute the Fv region. In some embodiments, the antigen-binding polypeptide of the antigen-binding molecule described herein comprises, or consists of, an Fv region which binds to an immune cell surface molecule. In some embodiments, the antigen-binding polypeptide comprises, or consists of, an Fv region which binds to a cancer cell antigen.

The VL and light chain constant (CL) region, and the VH region and heavy chain constant 1 (CH1) region of an antigen-binding region of an antibody together constitute the Fab region. In some embodiments, the antigen-binding polypeptide of the antigen-binding molecule described herein comprises, or consists of, a Fab region which binds to an immune cell surface molecule. In some embodiments, the antigen-binding polypeptide comprises, or consists of, a Fab region which binds to a cancer cell antigen.

In some embodiments, the antigen-binding polypeptide of the antigen-binding molecule described herein comprises, or consists of, a whole antibody which binds to an immune cell surface molecule. In some embodiments, the antigen-binding polypeptide of the antigen-binding molecule described herein comprises, or consists of, a whole antibody which binds to a cancer cell antigen. As used herein, “whole antibody” refers to an antibody having a structure which is substantially similar to the structure of an immunoglobulin (Ig). Different kinds of immunoglobulins and their structures are described e.g. in Schroeder and Cavacini J Allergy Clin Immunol. (2010) 125(202): S41-S52, which is hereby incorporated by reference in its entirety.

Immunoglobulins of type G (i.e. IgG) are −150 kDa glycoproteins comprising two heavy chains and two light chains. From N- to C-terminus, the heavy chains comprise a VH followed by a heavy chain constant region comprising three constant domains (CH1, CH2, and CH3), and similarly the light chain comprise a VL followed by a CL. Depending on the heavy chain, immunoglobulins may be classed as IgG (e.g. IgG1, IgG2, IgG3, IgG4), IgA (e.g. IgA1, IgA2), IgD, IgE, or IgM. The light chain may be kappa (κ) or lambda (λ).

In some embodiments, the antigen-binding polypeptide of the antigen-binding molecule described herein comprises, or consists of, an IgG (e.g. IgG1, IgG2, IgG3, IgG4), IgA (e.g. IgA1, IgA2), IgD, IgE, or IgM which binds to an immune cell surface molecule. In some embodiments, the antigen-binding polypeptide of the antigen-binding molecule described herein comprises, or consists of, an IgG (e.g. IgG1, IgG2, IgG3, IgG4), IgA (e.g. IgA1, IgA2), IgD, IgE, or IgM which binds to a cancer cell antigen.

In some embodiments, the antibody which binds to an immune cell surface molecule is an antibody capable of binding to CD3ε. In some embodiments, an antibody capable of binding to CD3ε is selected from an antibody, or CD3ε binding antibody fragment, disclosed in WO 2014/167022 A1 (e.g. clone H2C, clone CH2527), WO 2008/119565 A2, WO 2008/119566 A2 or WO 2008119567 A2 (e.g. clone A2J, clone I2C, clone E2M, clone F7O, clone G4H, clone H2C, clone E1L, clone F12Q, clone F6A, clone H1E), WO 2014/122143 A1 (e.g. clone SP34, BC-3), WO 2000/041474 A1 (clone UCHT1), WO 2007/033230 A2 (e.g. clone 28F11, clone 27H5, clone 23F10, clone 15C3), US 2016/0326249 A1, U.S. Pat. No. 7,635,472 B2, anti-CD3ε antibody clone 145-2C11 (Rockland Immunochemicals, Inc.), clone OKT3 (available e.g. from Novus Biologicals; described in Kjer-Nielsen et al., PNAS (2004) 101(20):7675-80), clone 4D10A6 (Abbiotec), clone HIT3a (Origene Technologies), clone OTI3E10 (Origene Technologies), clone CL1497 (Atlas Antibodies), clone CL1466 (Atlas Antibodies), clone RBT-CD3e (antibodies-online), clone CD3-12 (Aviva Systems Biology), clone CA17.2A12 (Aviva Systems Biology), clone 4E2 (Invitrogen Antibodies), clone FN-18 (Invitrogen Antibodies), clone 400 (Abbiotec), clone C3e/1308 (NSJ Bioreagents), clone CRIS-7 (NSJ Bioreagents), clone 500A2 (eBioscience), clone BB23-8E6 (Fitzgerald Industries International), clone PPT3 (Fitzgerald Industries International), clone CT-3 (Bio-Rad Antibodies), clone CD3-12 (Cell Signaling Technology), clone AT3H3 (IBL—America (Immuno-Biological Laboratories)), clone APA1/1 (BioLegend), clone 145-2C11 (MBL International), clone 301 (Sino Biological), clone MEM-92 (EXBIO Praha, A.S.), clone 3F3A1 (Proteintech Group Inc), clone REA613 (Miltenyi Biotec), clone HH3E (Dianova GmbH), clone MM0167-3F43 (Novus Biologicals), clone JXT3 (Acris Antibodies GmbH), clone SK7 (STEMCELL Technologies, Inc.), clone CA-3 (Bosterbio), clone 33-2A3 (Abcam), clone B-B11 (Abcam), clone 13B131 (United States Biological), clone MEM-57 (Enzo Life Sciences, Inc.), clone TR66 (Enzo Life Sciences, Inc.), clone LE-CD3 (Santa Cruz Biotechnology, Inc.), clone A9 (Santa Cruz Biotechnology, Inc.), clone KT3 (ThermoFisher Scientific), clone 611 B3 (Creative Diagnostics).

In some embodiments, the antibody which binds to a cancer cell antigen is an antibody capable of binding to mortalin. In some embodiments, an antibody capable of binding to mortalin is selected from an antibody, or mortalin-binding antibody fragment, disclosed in U.S. Pat. No. 8,591,893 B2 or WO 2006/022344 A1 (e.g. clone 52-3, clone 37-6, clone 37-1, clone 38-4, clone 38-5, clone 71-1 or clone 96-5), WO 2016/120325 A1 (e.g. cmHsp70.1, also described in Stangl et al. PNAS USA. (2011) 108(2):733-8), WO2011/071099 A1 or U.S. Pat. No. 8,586,042 B2 (e.g. hybridoma strain C-26 (FERM P-21875) or hybridoma strain C-69 (FERM P-21876)), Multhoff and Hightower, Cell Stress and Chaperones (2011) 16:251-255, or the anti-mortalin antibody clone 9F8 (Novus Biologicals), clone S52A-42 (Rockland Immunochemicals, Inc.), clone 30A5 (MBL International), clone 419605 (R&D Systems), clone 10D7 (GeneTex), clone 10F705 (United States Biological), or clone D-9 (Santa Cruz Biotechnology, Inc.).

In some embodiments, the antigen-binding molecule described herein comprises an antigen-binding polypeptide which is capable of binding to CD3ε. In some embodiments, the antigen-binding molecule comprises a CD3ε-binding domain comprising the amino acid sequences i) to vi):

(SEQ ID NO: 26)
i) LC-CDR1: RASSSVSYMN
(SEQ ID NO: 27)
ii) LC-CDR2: DTSKVAS
(SEQ ID NO: 28)
iii) LC-CDR3: QQWSSNPLT
(SEQ ID NO: 30)
iv) HC-CDR1: RYTMH
(SEQ ID NO: 31)
v) HC-CDR2: YINPSRGYTNYNQKFKD
(SEQ ID NO: 32)
vi) HC-CDR3: YYDDHYCLDY;

or a variant thereof in which one or two or three amino acids in one or more of the sequences i) to vi) are replaced with another amino acid.

In some embodiments, the antigen-binding molecule comprises a CD3ε-binding domain comprising:
a variable light chain (VL) region comprising, or consisting of, an amino acid sequence having at least 70% sequence identity to the amino acid sequence of SEQ ID NO:25; and
a variable heavy chain (VH) region comprising, or consisting of, an amino acid sequence having at least 70% sequence identity to the amino acid sequence of SEQ ID NO:29.

The CD3ε-binding domain may comprise an amino acid sequence having at least 70%, more preferably one of at least 75%, 80%, 85%, 86%, 87%, 88%, 89%, 90%, 91%, 92%, 93%, 94%, 95%, 96%, 97%, 98%, 99%, or 100%, sequence identity to the SEQ ID NO:25. The CD3ε-binding domain may comprise an amino acid sequence having at least 70%, more preferably one of at least 75%, 80%, 85%, 86%, 87%, 88%, 89%, 90%, 91%, 92%, 93%, 94%, 95%, 96%, 97%, 98%, 99%, or 100%, sequence identity to the SEQ ID NO:29.

In some embodiments, the antigen-binding molecule described herein comprises an antigen-binding polypeptide which is capable of binding to mortalin. In some embodiments, the antigen-binding molecule comprises a mortalin-binding domain comprising the amino acid sequences i) to vi):

(SEQ ID NO: 18)
i) LC-CDR1: KSSQSLLDSDGKTYLN
(SEQ ID NO: 19)
ii) LC-CDR2: LVSKLDS
(SEQ ID NO: 20)
iii) LC-CDR3: WQGTHFPRT
(SEQ ID NO: 22)
iv) HC-CDR1: SYWMH
(SEQ ID NO: 23)
v) HC-CDR2: EIDPSDSYTKYNQKFKG
(SEQ ID NO:24)
vi) HC-CDR3: GDY;

or a variant thereof in which one or two or three amino acids in one or more of the sequences i) to vi) are replaced with another amino acid.

In some embodiments, the antigen-binding molecule comprises a mortalin-binding domain comprising:
a variable light chain (VL) region comprising, or consisting of, an amino acid sequence having at least 70% sequence identity to the amino acid sequence of SEQ ID NO:17; and
a variable heavy chain (VH) region comprising, or consisting of, an amino acid sequence having at least 70% sequence identity to the amino acid sequence of SEQ ID NO:21.

The mortalin-binding domain may comprise an amino acid sequence having at least 70%, more preferably one of at least 75%, 80%, 85%, 86%, 87%, 88%, 89%, 90%, 91%, 92%, 93%, 94%, 95%, 96%, 97%, 98%, 99%, or 100%, sequence identity to the SEQ ID NO:17. The mortalin-binding domain may comprise an amino acid sequence having at least 70%, more preferably one of at least 75%, 80%, 85%, 86%, 87%, 88%, 89%, 90%, 91%, 92%, 93%, 94%, 95%, 96%, 97%, 98%, 99%, or 100%, sequence identity to the SEQ ID NO:21.

The light chain and heavy chain CDRs may also be particularly useful in conjunction with a number of different framework regions. Accordingly, light and/or heavy chains having LC-CDRs1-3 or HCs-CDR1-3 as described herein may possess an alternative framework region. Suitable framework regions are well known in the art and are described for example in M. Lefranc & G. Lefranc (2001) “The Immunoglobulin FactsBook”, Academic Press, incorporated herein by reference.

Molecular biology techniques suitable for producing the polypeptides such as antigen-binding polypeptides as described herein are well known in the art, such as those set out in Sambrook et al., Molecular Cloning: A Laboratory Manual, New York: Cold Spring Harbor Press, 1989.

Polypeptides may be expressed from a nucleotide sequence. The nucleotide sequence may be contained in a vector present in a cell, or may be incorporated into the genome of the cell.

A “vector” as used herein refers to a nucleic acid used as a vehicle to transfer exogenous genetic material into a cell. A vector may be an expression vector for expression of the genetic material in the cell. Such vectors may include a promoter sequence operably linked to the nucleotide sequence encoding the gene sequence to be expressed. A vector may also include a termination codon and expression enhancers. Any suitable vectors, promoters, enhancers and termination codons known in the art may be used to express polypeptides from a vector. Suitable vectors include plasmids, binary vectors, viral vectors and artificial chromosomes (e.g. yeast artificial chromosomes). The term “operably linked” may include the situation where a selected nucleotide sequence and regulatory nucleotide sequence (e.g. promoter and/or enhancer) are covalently linked in such a way as to place the expression of the nucleotide sequence under the influence or control of the regulatory sequence (thereby forming an expression cassette). Thus a regulatory sequence is operably linked to the selected nucleotide sequence if the regulatory sequence is capable of effecting transcription of the nucleotide sequence. Where appropriate, the resulting transcript may then be translated into a desired protein or polypeptide.

Any cell suitable for the expression of polypeptides may be used for producing polypeptides according to the present disclosure. The cell may be a prokaryote or eukaryote. Suitable prokaryotic cells include E. coli. Examples of eukaryotic cells include a yeast cell, a plant cell, insect cell or a mammalian cell (e.g. Chinese Hamster Ovary (CHO) cells). In some cases the cell is not a prokaryotic cell because some prokaryotic cells do not allow for the same post-translational modifications as eukaryotes. In addition, very high expression levels are possible in eukaryotes and proteins can be easier to purify from eukaryotes using appropriate tags. Specific plasmids may also be utilised which enhance secretion of the protein into the media.

Methods of producing a polypeptide of interest may involve culture or fermentation of a cell modified to express the polypeptide. The culture or fermentation may be performed in a bioreactor provided with an appropriate supply of nutrients, air/oxygen and/or growth factors. Secreted polypeptides can be collected by partitioning culture media/fermentation broth from the cells, extracting the protein content, and separating individual proteins to isolate secreted polypeptide. Culture, fermentation and separation techniques are well known to those of skill in the art. Bioreactors include one or more vessels in which cells may be cultured. Culture in the bioreactor may occur continuously, with a continuous flow of reactants into, and a continuous flow of cultured cells from, the reactor. Alternatively, the culture may occur in batches. The bioreactor monitors and controls environmental conditions such as pH, oxygen, flow rates into and out of, and agitation within the vessel such that optimum conditions are provided for the cells being cultured.

Following culture of cells that express the polypeptide of interest, that polypeptide is preferably isolated. Any suitable method for separating polypeptides from cell culture known in the art may be used. In order to isolate a polypeptide of interest from a culture, it may be necessary to first separate the cultured cells from media containing the polypeptide of interest. If the polypeptide of interest is secreted from the cells, the cells may be separated from the culture media that contains the secreted polypeptide by centrifugation. If the polypeptide of interest collects within the cell, it will be necessary to disrupt the cells prior to centrifugation, for example using sonification, rapid freeze-thaw or osmotic lysis. Centrifugation will produce a pellet containing the cultured cells, or cell debris of the cultured cells, and a supernatant containing culture medium and the polypeptide of interest. It may then be desirable to isolate the polypeptide of interest from the supernatant or culture medium, which may contain other protein and non-protein components. A common approach to separating polypeptide components from a supernatant or culture medium is by precipitation. Polypeptides of different solubility are precipitated at different concentrations of precipitating agent such as ammonium sulfate. For example, at low concentrations of precipitating agent, water soluble proteins are extracted. Thus, by adding increasing concentrations of precipitating agent, proteins of different solubility may be distinguished. Dialysis may be subsequently used to remove ammonium sulfate from the separated proteins. Other methods for distinguishing different polypeptides are known in the art, for example ion exchange chromatography and size chromatography. These may be used as an alternative to precipitation, or may be performed subsequently to precipitation.

Once the polypeptide of interest has been isolated from culture it may be necessary to concentrate the protein. A number of methods for concentrating a protein of interest are known in the art, such as ultrafiltration or lyophilisation.

Antigen-Binding Molecules Comprising an Aptamer and an Antigen-Binding Polypeptide

The antigen-binding molecules described herein comprise at least (i) an aptamer moiety and (ii) an antigen-binding polypeptide moiety. The aptamer and an antigen-binding polypeptide components of the antigen-binding molecules described herein may be covalently or non-covalently associated with one another.

It will be appreciated that the aptamer and antigen-binding polypeptide can be associated through any suitable association provided that the antigen-binding molecule retains the capability to bind to the cancer cell antigen and the immune cell surface molecule.

The association can be direct or indirect. For example, a conjugate between a first moiety (e.g. —NH2, —COOH, —N-hydroxysuccinimide, or -maleimide) and a second moiety (e.g., sulfhydryl, sulfur-containing amino acid) provided herein can be direct, e.g., by covalent bond or linker, or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like).

In some embodiments, the aptamer and the antigen-binding polypeptide may be linked by conjugation. That is, in some embodiments the antigen-binding molecule may be a conjugate of an aptamer and an antigen-binding polypeptide.

In some embodiments, conjugates are formed using conjugate chemistry including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

In some embodiments, the aptamer and antigen-binding polypeptide are non-covalently attached through a non-covalent chemical linker or covalent chemical linker formed by a reaction between a component of the aptamer and a component of the antigen-binding polypeptide. In some embodiments, the aptamer or antigen-binding polypeptide includes one or more reactive moieties, e.g., a covalent reactive moiety, as described herein (e.g., alkyne, azide, amine, ester, N-hydroxy-succinimide, maleimide or thiol reactive moiety). In some embodiments, the aptamer or antigen-binding polypeptide includes a linker comprising one or more reactive moieties, e.g., a covalent reactive moiety, as described herein (e.g., alkyne, azide, amine, ester, N-hydroxy-succinimide, maleimide or thiol reactive moiety

Useful reactive moieties or functional groups used for conjugate chemistries herein include, for example: (a) carboxyl groups and various derivatives thereof including, but not limited to, Nhydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc. (c) halo alkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold; (h) amine or sulfhydryl groups, which can be, for example, acylated, alkylated or oxidized; (i) alkenes, which can undergo, for example, cycloadditions, acylation, Michael addition, etc; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g. phosphines) to form, for example, phosphate diester bonds; and (n) sulfones, for example, vinyl sulfone.

The reactive functional groups can be chosen such that they do not participate in, or interfere with, the chemical stability of the targets described herein (i.e. the cancer cell antigen and/or immune cell surface molecule). By way of example, the aptamers may include a vinyl sulfone or other reactive moiety. Optionally, the aptamers can include a reactive moiety having the formula S—S—R. R can be, for example, a protecting group. Optionally, R is hexanol. As used herein, the term hexanol includes compounds with the formula C6H130H and includes, 1-hexanol, 2-hexanol, 3-hexanol, 2-methyl-1-pentanol, 3-methyl-1-pentanol, 4-methyl-1-pentanol, 2-methyl-2-pentanol, 3-methyl-2-pentanol, 4-methyl-2-pentanol, 2-methyl-3-pentanol, 3-methyl-3-pentanol, 2,2-dimethyl-1-butanol, 2,3-dimethyl-1-butanol, 3,3-dimethyl-1-butanol, 2,3-dimethyl-2-butanol, 3,3-dimethyl-2-butanol, and 2-ethyl-1-butanol. Optionally, R is 1-hexanol.

In some embodiments, conjugation of the aptamer and the antigen-binding polypeptide is achieved via click chemistry. Click chemistry and its use to produce conjugates of biomolecules is described in Nwe and Brechbiel Cancer Biother Radiopharm. (2009) 24(3):289-302, which is hereby incorporated by reference in its entirety.

Click chemistry reactions include cycloadditions and additions to double or triple bonds. More specific examples include [3+2] cycloadditions, such as the Cu(I)-catalyzed stepwise Huisgen 1,3-dipolar cycloaddition, thiol-alkene and thiol alkyne click reactions, Diels-Alder reactions and inverse electron demand Diels-Alder reactions, [4+1] cycloadditions, nucleophilic substitution especially to small strained rings, non-aldol type carbonyl reactions, such as the formation of ureas, and Michael Additions. Other examples include native chemical ligation of a thiolate group of an N-terminal cysteine with a C-terminal thioester, hydrazide formation by reaction of an aldehyde with a hydrazine, or oxime formation by reaction of an aldehyde with an aminooxy derivative, e.g. a hydroxylamine.

The aptamer and antigen-binding polypeptide may each comprise a member of a functional group pair for conjugation by click chemistry. Suitable functional group pairs include: an azide and an alkyne; a thiol and an alkene; a thiol and an alkyne; a diene and a dienophile; an isonitrile and a tetrazine; an epoxy and an aziridine; an amine and an isocyanate, an active methylene and an activated olefin, a thioester and a thiol; an aldehyde and a hydrazine; an aldehyde and an aminoxy derivative. Examples of each functional group suitable for the Click chemistry reactions are known in the art.

A particularly preferred functional group pair is an azide and an alkyne. Azides and alkynes may be used in the Huisgen 1,3-dipolar cycloaddition. The reaction forms a 1,2,3-triazole link. The alkyne may be a terminal or internal alkyne. The reaction of the azide with the alkyne may be catalysed. For example, the reaction may be a Copper(I)-catalyzed azide-alkyne cycloaddition (CuAAC), a Ruthenium catalysed 1,3-dipolar azide-alkyne cycloaddition (RuAAC) or a Silver(I) catalyzed azide-alkyne cycloaddition (Ag AAC). Alternatively, the reaction may be uncatalysed, e.g. where the alkyne is cylooctyne. The reaction conditions for such reactions are known in the art.

The choice of which member of a given functional group pair is provided on the aptamer, and which member of the pair is provided on the antigen-binding polypeptide is not particularly limited. The choice may be determined, for example, by ease of preparation of the aptamer and antigen-binding polypeptide, and/or the compatibility of the functional group with other functional groups in the aptamer and antigen-binding polypeptide. For example, an isocyanate may not be suitable as a functional group in the antigen-binding polypeptide because of potential inter- or intra-molecular reaction with amine groups of the polypeptide. The functional groups may be incorporated in the aptamer and/or antigen-binding peptides during synthesis.

An example of a molecule comprising an oligonucleotide and an antigen-binding polypeptide conjugated through a “click” reaction is provided in Gong et al., Bioconjugate Chem. (2016) 27(1): 217-225, which is hereby incorporated by reference in its entirety. Here, conjugation was achieved by alkyne-azide cycloaddition (the Cu-free click reaction), in which the antibody was prepared including a dibenzocyclooctyne (DBCO) moiety in the antibody constant region, and subsequently linked covalently with an azide-modified oligonucleotide.

In some embodiments, the aptamer and antigen-binding polypeptide are conjugated by a method employing 2,4-Dihydroxy-1,4-benzoxazinone (DIBOA). In some embodiments, the antigen-binding molecule described herein is prepared by a method essentially as described in Example 1.

Accordingly, in some embodiments the antigen-binding molecule described herein is produced by a method comprising: reducing the antigen-binding polypeptide, e.g. by treatment with a reducing agent (eg. tris(2-carboxyethyl)phosphine (TCEP)); optionally purifying the reduced antigen-binding polypeptide (e.g. from reducing agent); contacting the reduced antigen-binding polypeptide with Br—CH₂CO-DIBOA; optionally purifying the antigen-binding polypeptide-CH₂CO-DIBOA molecule; contacting the antigen-binding polypeptide-CH₂CO-DIBOA molecule with the aptamer; optionally purifying antigen-binding polypeptide-aptamer conjugate; optionally concentrating the antigen-binding polypeptide-aptamer conjugate.

In embodiments, the antigen-binding polypeptide is covalently bound to the aptamer using conjugate chemistry as described above, including, but not limited to, nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition).

In some embodiments the antigen-binding molecule described herein may be formed through association via specific binding partners. One of the pair of binding partners is attached to (or incorporated during synthesis of) the aptamer, and the other member of the pair of binding partners is attached to (or incorporated during synthesis of) the antigen-binding polypeptide. The aptamer and antigen-binding polypeptide then associate through the specific binding partners to form the antigen-binding molecule. Examples of suitable binding partner pairs include single stranded oligonucleotides having complementary sequences, and biotin and avidin/streptavidin.

In some embodiments, the antigen-binding molecule may comprise one or more linker sequences between the aptamer and the antigen-binding polypeptide. The linker sequence may comprise, or consist of, a sequence of amino acids (i.e. may be a peptide linker). The linker sequence may comprise, or consist of, a sequence of nucleotides (i.e. may be oligonucleotide linker). The linker sequence may comprise, or consist of, a hydrocarbon spacer.

Peptide linker sequences are known to the skilled person, and are described, for example in Chen et al., Adv Drug Deliv Rev (2013) 65(10): 1357-1369, hereby incorporated by reference. In some embodiments, the peptide linker sequence may be a flexible peptide linker sequence. In some embodiments, the peptide linker sequence may comprise 1-25, 1-20, 1-15, 1-10 or 1-5 amino acids. In some embodiments, a peptide linker sequence may comprise fewer than 25, 20, 15, 10 or 5 amino acids.

The oligonucleotide linker sequence may comprise 1-50, 1-40, 1-30, 1-25, 1-20, 1-15, 1-10 or 1-5 nucleotides. In some embodiments, an oligonucleotide linker sequence may comprise fewer than 50, 40, 30, 25, 20, 15, 10 or 5 nucleotides.

In some embodiments, the oligonucleotide linker sequence may participate in the formation of a non-covalent complex of the aptamer and antigen-binding polypeptide through the formation of a “sticky bridge”. A sticky bridge comprises complementary oligonucleotides. One member of the pair of complementary oligonucleotide sequences may be provided at the 3′ or 5′ end of the sequence of the aptamer, and the other member of the pair of complementary oligonucleotide sequences may be attached to the antigen-binding polypeptide. The complementary sticky sequences are allowed to hybridise and form a non-covalent complex comprising the aptamer and the antigen-binding polypeptide. The sticky sequence may be GC- or AU-rich, and each sticky sequence may comprise about 16 nucleotides, e.g. 14 to 20 nucleotides or one of 14, 15, 16, 17, 18, 19 or 20 nucleotides.

A hydrocarbon spacer may be an optionally substituted C₁₋₃₀ alkyl or optionally substituted C₂₋₃₀ alkenyl; or polyethylene glycol molecule(s). In some embodiments the hydrocardon spacer may be a polycarbon spacer, consistent with formation of a “sticky bridge”. The polycarbon spacer may be an optionally substituted C₁₀₋₃₀ alkyl, optionally substituted C₁₀₋₁₅ alkyl, optionally substituted C₁₅₋₂₀ alkyl, optionally substituted C₂₀₋₂₅ alkyl, optionally substituted C₂₅₋₃₀ alkyl, optionally substituted C₁₀₋₃₀ alkenyl, optionally substituted C₁₀₋₁₅ alkenyl, optionally substituted C₁₅₋₂₀ alkenyl, optionally substituted C₂₀₋₂₅ alkenyl, optionally substituted C₂₅₋₃₀ alkenyl.

The antigen-binding molecule described herein may additionally comprise a polycarbon linker, e.g. between a sticky bridge forming oligonucleotide linker and the aptamer. Such polycarbon linkers are useful for providing rigidity to the aptamer, decreasing the likelihood that the inclusion of the additional sticky sequences will interfere with proper aptamer folding (e.g. see Zhou et al. Nucleic Acids Res. 2009; 37(9):3094-3109). The polycarbon linker may comprise one or more of an oligonucleotide sequence, hydrocarbon spacer elements such as optionally substituted C₁₋₃₀ alkyl or optionally substituted C₂₋₃₀ alkenyl; or polyethylene glycol molecule(s). In some embodiments the polycarbon linker may be a polycarbon linker, consistent with formation of a “sticky bridge”. The polycarbon linker may be an optionally substituted C₁₀₋₃₀ alkyl, optionally substituted C₁₀₋₁₅ alkyl, optionally substituted C₁₅₋₂₀ alkyl, optionally substituted C₂₀₋₂₅ alkyl, optionally substituted C₂₅₋₃₀ alkyl, optionally substituted C₁₀₋₃₀ alkenyl, optionally substituted C₁₀₋₁₅ alkenyl, optionally substituted C₁₅₋₂₀ alkenyl, optionally substituted C₂₀₋₂₅ alkenyl, optionally substituted C₂₅₋₃₀ alkenyl.

A polycarbon linker may be incorporated between a member of the pair of complementary oligonucleotide sequences and the aptamer and/or between a member of the pair of complementary oligonucleotide sequences and the antigen-binding polypeptide.

In embodiments, the linker may include or have the structure below. In embodiments, the linker connects with the 3′ phosphate of the guanine on one end and the 5′ phosphate of the thymidine on the other end, and the nucleobases may be modified.

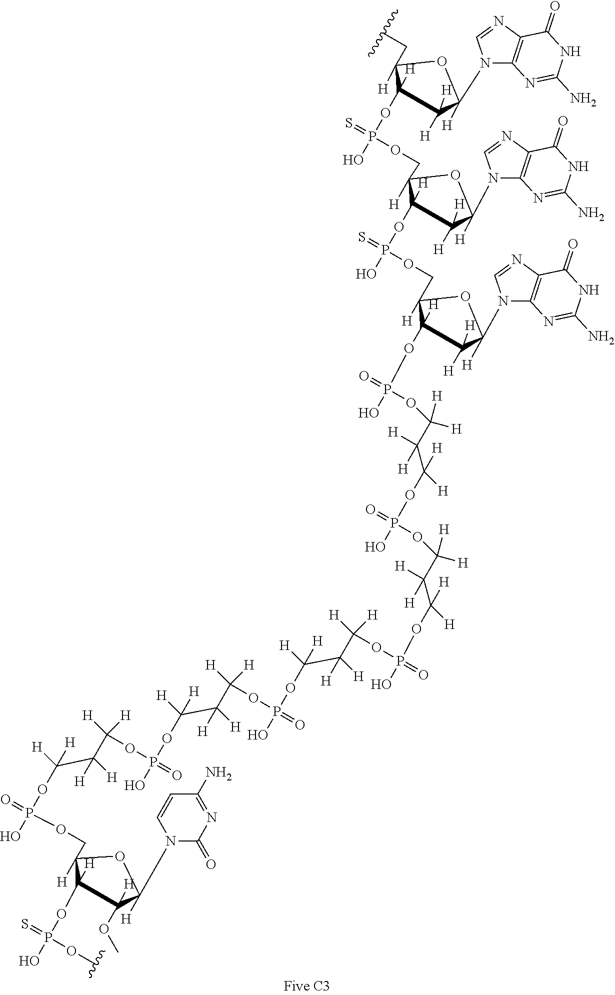

In embodiments, the above formula represents a portion of an aptamer linked at the 3′-OH end with a (CH₂)₃ linker, which links to the 5′-phosphate of the hybridization sequence (i.e. sticky bridge, sticky sequence).

The linker may be a bond, substituted (e.g. substituted with a substituent group, size-limited substituent group or lower substituent group) or unsubstituted alkylene, substituted (e.g. substituted with a substituent group, size-limited substituent group or lower substituent group) or unsubstituted heteroalkylene, substituted (e.g. substituted with a substituent group, size-limited substituent group or lower substituent group) or unsubstituted cycloalkylene, substituted (e.g. substituted with a substituent group, size-limited substituent group or lower substituent group) or unsubstituted heterocycloalkylene, substituted (e.g. substituted with a substituent group, size-limited substituent group or lower substituent group) or unsubstituted arylene, substituted (e.g. substituted with a substituent group, size-limited substituent group or lower substituent group) or unsubstituted heteroarylene, substituted (e.g. substituted with a substituent group, size-limited substituent group or lower substituent group) or unsubstituted cyclo-heteroalkylene or —(CH₂)ₙ—PO₄—[(CH₂)ₙ—PO₄]_{z}—(CH₂)ₙ, in which the symbol n is an integer from 1 to 5 (e.g., 3) and the symbol z is an integer from 0 to 50 (e.g. from 0 to 25, 0 to 10, or 0 to 5). In embodiments, n is 3 and z is 0 to 5 or 1 to 5. In embodiments, n is 3 and z is 0 to 4 or 1 to 4. In embodiments, n is 3 and z is 0 to 3 or 1 to 3. In embodiments, n is 3 and z is 3. Linkers may be added during the synthesis in sequence.

In embodiments, the linker is a covalent linker (i.e. a linker that covalently attaches at least two (e.g. 2) portions of a compound). In embodiments, the linker is or includes a substituted (e.g. substituted with a substituent group, size-limited substituent group or lower substituent group) or unsubstituted alkylene or heteroalkylene linker. In embodiments, heteroalkylene linkers are connected to each other with an intervening phosphate bond. In embodiments, the covalent linker is a substituted (e.g. substituted with a substituent group, size-limited substituent group or lower substituent group) or unsubstituted heteroalkylene linker.

In embodiments, the linker is a substituted (e.g. substituted with a substituent group, size-limited substituent group or lower substituent group) or unsubstituted heteroalkylene or substituted (e.g. substituted with a substituent group, size-limited substituent group or lower substituent group) or unsubstituted cyclo-heteroalkylene. A “cyclo-heteroalkylene,” as used herein is a heteroalkylene having a one or more divalent cyclic moieties within the heteroalkylene chain. The cyclic moiety may be a substituted (e.g. substituted with a substituent group, size-limited substituent group or lower substituent group) or unsubstituted cycloalklylene, substituted (e.g. substituted with a substituent group, size-limited substituent group or lower substituent group) or unsubstituted heterocycloalkylene, substituted (e.g. substituted with a substituent group, size-limited substituent group or lower substituent group) or unsubstituted arylene or substituted (e.g. substituted with a substituent group, size-limited substituent group or lower substituent group) or unsubstituted heteroarylene. In embodiments, the cyclic moiety is a substituted (e.g. substituted with a substituent group, size-limited substituent group or lower substituent group) or unsubstituted ribose (e.g., a nucleoside). In embodiments, the cyclic moiety serves as a branch point of the linker thereby forming a branched linker. The cyclic moiety branch point may be used to attach additional functional moieties to the conjugates provided herein, such as detectable moieties, drug moieties or biomolecule.

In embodiments, the linker includes one or more C3 linkers as set forth in the above structure. In embodiment, a C3 linker is or contains a —(CH₂)ₙ—PO₄— moiety. In embodiments, the linker is or contains a moiety having the formula:

—(CH₂)ₙ—PO₄—[(CH₂)ₙ—PO₄]_{z}—(CH₂)ₙ—.

In the formula above, the symbol n is an integer from 1 to 5 (e.g., 3) and the symbol z is an integer from 0 to 50 (e.g. from 0 to 25, 0 to 10, or 0 to 5). In embodiments, n is 3 and z is 0 to 5 or 1 to 5. In embodiments, n is 3 and z is 0 to 4 or 1 to 4. In embodiments, n is 3 and z is 0 to 3 or 1 to 3. In embodiments, n is 3 and z is 3.

For example, the linker may have the structure below, where the linker connects with the 3′ phosphate of the guanine on one end and the 5′ phosphate of the thymidine on the other end:

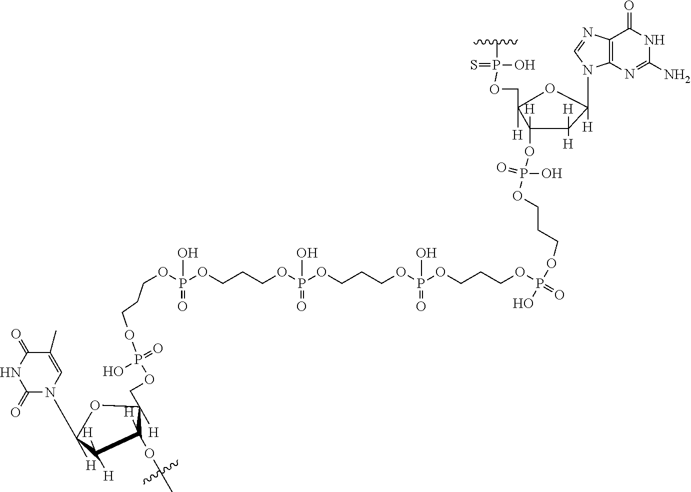

In embodiments, linker may include an unsubstituted C₃ heteroalkylene.

In embodiments, linker may include an unsubstituted C₆ to C₁₂ heteroalkylene.

In embodiments, the linker includes an unsubstituted C₃ alkylene (e.g. as described above). In embodiments, the linker may be unsubstituted Cis alkylene. In embodiments, the linker includes an unsubstituted C₆ to C₁₆ alkylene. In embodiments, the linker may be a substituted (e.g. substituted with a substituent group, size-limited substituent group or lower substituent group) or unsubstituted alkylene, substituted (e.g. substituted with a substituent group, size-limited substituent group or lower substituent group) or unsubstituted heteroalkylene, substituted (e.g. substituted with a substituent group, size-limited substituent group or lower substituent group) or unsubstituted cycloalkylene, substituted (e.g. substituted with a substituent group, size-limited substituent group or lower substituent group) or unsubstituted heterocycloalkylene, substituted (e.g. substituted with a substituent group, size-limited substituent group or lower substituent group) or unsubstituted arylene, or substituted (e.g. substituted with a substituent group, size-limited substituent group or lower substituent group) or unsubstituted heteroarylene. In embodiments, the linker may be a substituted (e.g. substituted with a substituent group, size-limited substituent group or lower substituent group) or unsubstituted C₁-C₄₀ alkylene, substituted (e.g. substituted with a substituent group, size-limited substituent group or lower substituent group) or unsubstituted 2 to 40 membered heteroalkylene, substituted (e.g. substituted with a substituent group, size-limited substituent group or lower substituent group) or unsubstituted C₃-C₈ cycloalkylene, substituted (e.g. substituted with a substituent group, size-limited substituent group or lower substituent group) or unsubstituted 3 to 8 membered heterocycloalkylene, substituted (e.g. substituted with a substituent group, size-limited substituent group or lower substituent group) or unsubstituted C₆-C₁₀ arylene, or substituted (e.g. substituted with a substituent group, size-limited substituent group or lower substituent group) or unsubstituted 5 to 10 membered heteroarylene. In embodiments, the linker may be an unsubstituted C₁-C₄₀ alkylene, unsubstituted 2 to 40 membered heteroalkylene, unsubstituted C₃-C₈ cycloalkylene, unsubstituted 3 to 8 membered heterocycloalkylene, unsubstituted C₆-C₁₀ arylene, or unsubstituted 5 to 10 membered heteroarylene. In embodiments, the linker may be a substituted (e.g. substituted with a substituent group, size-limited substituent group or lower substituent group) 2 to 40 membered heteroalkylene.

A linker may be a bond, nucleic acid sequence, two nucleic acid sequences, DNA sequence, two DNA sequences, nucleic acid analog sequence, substituted (e.g. substituted with a substituent group, size-limited substituent group or lower substituent group) or unsubstituted alkylene, substituted (e.g. substituted with a substituent group, size-limited substituent group or lower substituent group) or unsubstituted heteroalkylene, substituted (e.g. substituted with a substituent group, size-limited substituent group or lower substituent group) or unsubstituted cycloalkylene, substituted (e.g. substituted with a substituent group, size-limited substituent group or lower substituent group) or unsubstituted heterocycloalkylene, substituted (e.g. substituted with a substituent group, size-limited substituent group or lower substituent group) or unsubstituted arylene, or substituted (e.g. substituted with a substituent group, size-limited substituent group or lower substituent group) or unsubstituted heteroarylene.

In embodiments, the linker is or contains a substituted (e.g. substituted with a substituent group, size-limited substituent group or lower substituent group) or unsubstituted alkylene, substituted (e.g. substituted with a substituent group, size-limited substituent group or lower substituent group) or unsubstituted heteroalkylene, substituted (e.g. substituted with a substituent group, size-limited substituent group or lower substituent group) or unsubstituted cycloalkylene, substituted (e.g. substituted with a substituent group, size-limited substituent group or lower substituent group) or unsubstituted heterocycloalkylene, substituted (e.g. substituted with a substituent group, size-limited substituent group or lower substituent group) or unsubstituted arylene, or substituted (e.g. substituted with a substituent group, size-limited substituent group or lower substituent group) or unsubstituted heteroarylene. In embodiments, the linker is a substituted (e.g. substituted with a substituent group, size-limited substituent group or lower substituent group) or unsubstituted C₁-C₂₀ alkylene, substituted (e.g. substituted with a substituent group, size-limited substituent group or lower substituent group) or unsubstituted 2 to 20 membered heteroalkylene, substituted (e.g. substituted with a substituent group, size-limited substituent group or lower substituent group) or unsubstituted C₃-C₈ cycloalkylene, substituted (e.g. substituted with a substituent group, size-limited substituent group or lower substituent group) or unsubstituted 3 to 8 membered heterocycloalkylene, substituted (e.g. substituted with a substituent group, size-limited substituent group or lower substituent group) or unsubstituted C₆-C₁₀ arylene, or substituted (e.g. substituted with a substituent group, size-limited substituent group or lower substituent group) or unsubstituted 5 to 10 membered heteroarylene. In embodiments, the linker is an unsubstituted C1-C₂₀ alkylene, unsubstituted 2 to 20 membered heteroalkylene, unsubstituted C₃-C₈ cycloalkylene, unsubstituted 3 to 8 membered heterocycloalkylene, unsubstituted C₆-C₁₀ arylene, or unsubstituted 5 to 10 membered heteroarylene. In embodiments, the linker is an unsubstituted C₁-C₂₀ alkylene. In embodiments, the linker is a substituted (e.g. substituted with a substituent group, size-limited substituent group or lower substituent group) or unsubstituted C₁-C₄₀ alkylene, substituted (e.g. substituted with a substituent group, size-limited substituent group or lower substituent group) or unsubstituted 2 to 40 membered heteroalkylene, substituted (e.g. substituted with a substituent group, size-limited substituent group or lower substituent group) or unsubstituted C₃-C₈ cycloalkylene, substituted (e.g. substituted with a substituent group, size-limited substituent group or lower substituent group) or unsubstituted 3 to 8 membered heterocycloalkylene, substituted (e.g. substituted with a substituent group, size-limited substituent group or lower substituent group) or unsubstituted C₆-C₁₀ arylene, or substituted (e.g. substituted with a substituent group, size-limited substituent group or lower substituent group) or unsubstituted 5 to 10 membered heteroarylene. In embodiments, the linker is a substituted (e.g. substituted with a substituent group, size-limited substituent group or lower substituent group) or unsubstituted C₁-C₄₀ alkylene. In embodiments, the linker is a substituted (e.g. substituted with a substituent group, size-limited substituent group or lower substituent group) or unsubstituted 2 to 40 membered heteroalkylene. In embodiments, the linker is a substituted (e.g. substituted with a substituent group, size-limited substituent group or lower substituent group) 2 to 40 membered heteroalkylene. In embodiments, the linker includes alkyl phosphates (e.g., propyl phosphates). In embodiments, the linker has alkyl phosphates (e.g., propyl phosphates) bonded to the reminder of the compound by phosphates at both ends. In embodiments, the linker has 1-5 alkyl phosphates (e.g., propyl phosphates) bonded to the reminder of the compound by phosphates at both ends. In embodiments, the linker has 1-4 alkyl phosphates (e.g., propyl phosphates) bonded to the reminder of the compound by phosphates at both ends. In embodiments, the linker has 4 alkyl phosphates (e.g., propyl phosphates) bonded to the reminder of the compound by phosphates at both ends. A person having ordinary skill in the art will recognize that a linker having alkyl phosphates that is bonded to the remainder of the compound by phosphates on both ends will have one more phosphate than alkylene groups (e.g., a linker having 4 alkyl phosphates that is bonded to the reminder of the compound by phosphates at both ends will have five phosphates and four alkyl groups with alternating phosphate groups and alkyl groups).

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

The symbol “ [symbol] ” denotes the point of attachment of a chemical moiety to the remainder of a molecule or chemical formula.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —Cl₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl, heteroalkyl, cycloalkyl, heterocycloalkyl, aryl, heteroaryl, substituted with at least one substituent selected from:
   (i) oxo, halogen, —CCl₃, —CBr₃, —CF₃, —Cl₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl, heteroalkyl, cycloalkyl, heterocycloalkyl, aryl, heteroaryl, substituted with at least one substituent selected from:
      (a) oxo, halogen, —CCl₃, —CBr₃, —CF₃, —Cl₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl, heteroalkyl, cycloalkyl, heterocycloalkyl, aryl, heteroaryl, substituted with at least one substituent selected from: oxo, halogen, —CCl₃, —CBr₃, —CF₃, —Cl₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

The term “oxo,” as used herein, means an oxygen that is double bonded to a carbon atom.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

As used herein, the term “isomers” refers to compounds having the same number and kind of atoms, and hence the same molecular weight, but differing in respect to the structural arrangement or configuration of the atoms.

Also provided herein is a method for producing an antigen-binding molecule described herein. Generally, the method comprises covalently or non-covalently associating an aptamer as described herein and an antigen-binding polypeptide as described herein to form an antigen-binding molecule described herein. In some embodiments the methods comprise preparing an aptamer as described herein. In some embodiments the methods comprise preparing an antigen-binding polypeptide as described herein.

Antigen-binding molecules described herein may additionally comprise one or more additional moieties for use of the antigen-binding molecules in therapeutic, prophylactic, diagnostic and/or prognostic applications.

The antigen-binding molecules may be used to deliver molecules useful in therapeutic/prophylactic and/or diagnostic/prognostic applications. The molecule may form part of the antigen-binding molecule described herein. In some embodiments, the therapeutic, prophylactic, diagnostic and/or prognostic molecule (e.g. therapeutic moiety, imaging moiety etc.) is covalently or non-covalently associated with the antigen-binding molecule.

The antigen-binding molecule may be used to deliver a therapeutic, prophylactic, diagnostic and/or prognostic molecule to a target cell or tissue. The therapeutic, prophylactic, diagnostic and/or prognostic molecule may be internalised by the target cell.

Where the antigen-binding molecule described herein comprises one or more therapeutic, prophylactic, diagnostic and/or prognostic molecules, the molecule(s) may be covalently (e.g. directly or through a covalently-bonded intermediary) attached to the aptamer and/or antigen-binding polypeptide, e.g. as described hereinabove. In some embodiments, the molecule(s) may be non-covalently (e.g. through ionic bond(s), van der Waal's bond(s)/interactions, hydrogen bond(s), polar bond(s), or combinations or mixtures thereof) attached to the aptamer and/or antigen-binding polypeptide.

In some embodiments the molecule is a therapeutic/prophylactic moiety. The therapeutic/prophylactic moiety provides therapeutic benefit (prevention, eradication, amelioration of a disease/condition to be treated/prevented) when given to a subject in need thereof. Therapeutic/prophylactic moieties as provided herein may include, without limitation, peptides, proteins, nucleic acids (e.g. antisense nucleic acids, e.g. miRNA, siRNA, saRNA), nucleic acid analogs, small molecules, immunostimulators, antibodies, enzymes (e.g. nucleases, e.g. zinc finger nucelases, transcription activator-like effector nucleases, Cas-9), prodrugs, cytotoxic agents (e.g. toxins) including, but not limited to ricin, doxorubicin, daunorubicin, taxol, ethidium bromide, mitomycin, etoposide, tenoposide, vincristine, vinblastine, colchicine, dihydroxy anthracin dione, actinomycin D, diphteria toxin, Pseudomonas exotoxin (PE) A, PE40, abrin, and glucocorticoid. In some embodiments, the therapeutic/prophylactic moiety is an anti-cancer agent or chemotherapeutic agent (e.g. gemcitabine) as described herein.

In some embodiments the molecule is a detectable moiety. In some embodiments the detectable moiety is a detectable spectroscopic, photochemical, biochemical, immunochemical, chemical, or other physical means. Exemplary detectable moieties include, without limitation, ³²P, radionuclides, positron-emitting isotopes, fluorescent dyes, fluorophores, antibodies, bioluminescent molecules, chemoluminescent molecules, photoactive molecules, metals, electron-dense reagents, enzymes (e.g., as commonly used in an ELISA), magnetic contrast agents, quantum dots, nanoparticles, biotin, digoxigenin, haptens and proteins or other entities which can be made detectable, e.g., by incorporating a radiolabel into a peptide or antibody specifically reactive with a target antigen. Any method known in the art for conjugating an antibody to the moiety may be employed, e.g., using methods described in Hermanson, Bioconjugate Techniques 1996, Academic Press, Inc., San Diego. Exemplary fluorophores include fluorescein, rhodamine, GFP, coumarin, FITC, Alexa fluor, Cy3, Cy5, BODIPY, and cyanine dyes. Exemplary radionuclides include Fluorine-18, Gallium-68, and Copper-64. Exemplary magnetic contrast agents include gadolinium, iron oxide and iron platinum, and manganese. In some embodiments, the imaging moiety is a bioluminescent molecule.

Functional Properties of the Antigen-Binding Molecules

The antigen-binding molecule described herein may be characterised by reference to certain functional properties. In some embodiments, the antigen-binding molecule described herein may possess one or more of the following properties:
binds to a cancer cell antigen;
binds to an immune cell surface molecule;
binds to cancer cell antigen-expressing cells;
binds to immune cell surface molecule-expressing cells;
increases/enhances killing of cancer cell antigen-expressing cells by immune cell surface molecule-expressing cells (e.g. in an in vitro assay);
inhibits the development/progression of a cancer (e.g. in vivo); and inhibits the proliferation and/or survival of cells of a cancer.

It will be appreciated that “a cancer cell antigen” refers to a cancer cell antigen according to any embodiment described herein, and that likewise “an immune cell surface molecule” refers to an immune cell surface molecule according to any embodiment described herein.

The antigen-binding molecule described herein preferably retains the target-binding properties of its constituent aptamer and antigen-binding polypeptide components. The antigen-binding molecule described herein is multispecific, i.e. displays specific binding to more than one target. In particular, the antigen-binding molecule is binding to a cancer cell antigen and an immune cell surface molecule, and so is at least bispecific. The term “bispecific” means that the antigen-binding molecule is able to bind specifically to at least two distinct antigenic determinants.

The antigen-binding molecule described herein displays at least monovalent binding with respect to the cancer cell antigen, and also displays at least monovalent binding with respect to the immune cell surface molecule. Binding valency refers to the number of binding sites in an antigen-binding molecule for a given antigenic determinant. For example, the molecule chBiTE represented graphically in FIG. 1 is monovalent with respect to binding to mortalin, and bivalent with respect to binding to CD3ε.

In some embodiments the antigen-binding molecule comprises one binding site for a cancer cell antigen, and one binding site for an immune cell surface molecule. In some embodiments the antigen-binding molecule comprises one binding site for a cancer cell antigen, and two binding sites for an immune cell surface molecule. In some embodiments the antigen-binding molecule comprises two binding sites for a cancer cell antigen, and one binding site for an immune cell surface molecule.

In some embodiments the antigen-binding molecule described herein is capable of simultaneously binding the cancer cell antigen and the immune cell surface molecule, particularly wherein the cancer cell antigen and the immune cell surface molecule are expressed on different cells.

The antigen-binding molecule which is capable of binding to a cancer cell antigen and an immune cell surface molecule preferably binds the cancer cell antigen and the immune cell surface molecule with greater affinity, and/or with greater duration than it binds to proteins other than the cancer cell antigen or the immune cell surface molecule (i.e. non-targets).

For example, where the cancer cell antigen is mortalin, the antigen-binding molecule may bind to mortalin with greater affinity as compared to the affinity of binding to one or more other members of the heat shock protein 70 family, e.g. Hsp70-1a, Hsp70-1b, Hsp70-1t, Hsp70-2, Hsp70-5, Hsp70-6, or Hsc70. For example, where the immune cell surface molecule is CD3ε the antigen-binding molecule may bind to CD3ε with greater affinity as compared to the affinity of binding to one or more other CD3 polypeptides, e.g. CD3δ, CD3γ, CD3ζ or CD3η.

In some embodiments, the extent of binding of an antigen-binding molecule to an non-target is less than about 10% of the binding of the antibody to the target as measured, e.g., by ELISA, SPR, Bio-Layer Interferometry (BLI), MicroScale Thermophoresis (MST), or by a radioimmunoassay (RIA). Alternatively, the binding specificity may be reflected in terms of binding affinity, where the antigen-binding molecule described herein binds to the cancer cell antigen and/or the immune cell surface molecule with an affinity that is at least 0.1 order of magnitude greater than the affinity towards a non-target molecule. In some embodiments, the antigen-binding molecule described herein binds to the cancer cell antigen and/or the immune cell surface molecule with an affinity that is one of at least 0.2, 0.3, 0.4, 0.5, 0.6, 0.7, 0.8, 0.9, 1.0, 1.5, or 2.0 orders of magnitude greater than the affinity towards another, non-target molecule.

Binding affinity of an antigen-binding molecule for its target is often described in terms of its dissociation constant (K_{D}). Binding affinity can be measured by methods known in the art, such as by ELISA, Surface Plasmon Resonance (SPR; see e.g. Hearty et al., Methods Mol Biol (2012) 907:411-442; or Rich et al., Anal Biochem. 2008 Feb. 1; 373(1):112-20), Bio-Layer Interferometry (see e.g. Lad et al., (2015) J Biomol Screen 20(4): 498-507; or Concepcion et al., Comb Chem High Throughput Screen. 2009 September; 12(8):791-800), MicroScale Thermophoresis (MST) analysis (see e.g. Jerabek-Willemsen et al., Assay Drug Dev Technol. 2011 August; 9(4): 342-353), or by a radiolabelled antigen binding assay (RIA).

In some embodiments, the antigen-binding molecule described herein binds to the cancer cell antigen with a K_{D} of 10 μM or less, preferably one of ≤5 μM, 52 μM, ≤1 μM, ≤500 nM, ≤100 nM, ≤75 nM, ≤50 nM, ≤40 nM, ≤30 nM, ≤20 nM, ≤15 nM, ≤12.5 nM, ≤10 nM, 59 nM, 58 nM, 57 nM, 56 nM, ≤5 nM, 54 nM ≤3 nM, ≤2 nM, ≤1 nM, ≤500 μM. In some embodiments, the antigen-binding molecule described herein binds to the immune cell surface molecule with a K_{D} of 10 μM or less, preferably one of ≤5 μM, 52 μM, ≤1 μM, 5500 nM, ≤100 nM, ≤75 nM, ≤50 nM, ≤40 nM, ≤30 nM, ≤20 nM, ≤15 nM, ≤12.5 nM, ≤10 nM, ≤9 nM, ≤8 nM, ≤7 nM, ≤6 nM, ≤5 nM, ≤4 nM ≤3 nM, ≤2 nM, ≤1 nM, ≤500 μM.

In some embodiments, the antigen-binding molecule described herein binds to the cancer cell antigen with an affinity of binding (e.g. as determined by ELISA) of EC50=1000 ng/ml or less, preferably one of ≤900 ng/ml, ≤800 ng/ml, ≤700 ng/ml, ≤600 ng/ml, ≤500 ng/ml, ≤400 ng/ml, ≤300 ng/ml, ≤200 ng/ml, ≤100 ng/ml, 590 ng/ml, ≤80 ng/ml, ≤70 ng/ml, ≤60 ng/ml, ≤50 ng/ml, ≤40 ng/ml, ≤30 ng/ml, ≤20 ng/ml, ≤15 ng/ml, 510 ng/ml, ≤7.5 ng/ml, ≤5 ng/ml, ≤2.5 ng/ml, or ≤1 ng/ml. In some embodiments, the antigen-binding molecule described herein binds to the immune cell surface molecule with an affinity of binding (e.g. as determined by ELISA) of EC50=1000 ng/ml or less, preferably one of ≤900 ng/ml, 5800 ng/ml, ≤700 ng/ml, ≤600 ng/ml, ≤500 ng/ml, ≤400 ng/ml, ≤300 ng/ml, ≤200 ng/ml, ≤100 ng/ml, ≤90 ng/ml, ≤80 ng/ml, 570 ng/ml, ≤60 ng/ml, ≤50 ng/ml, ≤40 ng/ml, ≤30 ng/ml, ≤20 ng/ml, ≤15 ng/ml, ≤10 ng/ml, ≤7.5 ng/ml, 55 ng/ml, ≤2.5 ng/ml, or ≤1 ng/ml.

Affinity of binding to the cancer cell antigen and/or the immune cell surface molecule may be analysed in vitro by ELISA assay. Suitable assays are well known in the art and can be performed by the skilled person, for example, as described in Antibody Engineering, vol. 1 (2^{nd} Edn), Springer Protocols, Springer (2010), Part V, pp 657-665.

The antigen-binding molecule described herein preferably binds to the cancer cell antigen in a region of the cancer cell antigen which is accessible to an antigen-binding molecule (i.e., an extracellular antigen-binding molecule) when the cancer cell antigen is expressed at the cell surface (i.e. in or at the cell membrane). In some embodiments the antigen-binding molecule described herein is capable of binding to the cancer cell antigen when the cancer cell antigen is expressed at the cell surface. In some embodiments the antigen-binding molecule described herein is capable of binding to the cancer cell antigen when the cancer cell antigen is displayed on the external surface of a cell expressing the cancer cell antigen at the cell surface.

The antigen-binding molecule described herein preferably binds to the immune cell surface molecule in a region of the immune cell surface molecule which is accessible to an antigen-binding molecule (i.e., an extracellular antigen-binding molecule) when the immune cell surface molecule is expressed at the cell surface (i.e. in or at the cell membrane). In some embodiments the antigen-binding molecule described herein is capable of binding to the immune cell surface molecule when the immune cell surface molecule is expressed at the cell surface. In some embodiments the antigen-binding molecule described herein is capable of binding to the immune cell surface molecule when the immune cell surface molecule is displayed on the external surface of a cell expressing the immune cell surface molecule at the cell surface.

For example, where the cancer cell antigen is mortalin, the antigen-binding molecule may bind to mortalin-expressing cells, such as cells expressing mortalin at the cell surface, e.g. PANC-1, U251 and/or HCT116 cells. For example, where the immune cell surface molecule is a CD3-TCR complex polypeptide (e.g. CD3ε), the antigen-binding molecule may bind to cells expressing the CD3-TCR complex polypeptide, such as T cells.

The ability of an antigen-binding molecule to bind to a given cell type can be analysed by contacting cells with the antigen-binding molecule, and detecting antigen-binding molecule bound to the cells, e.g. after a washing step to remove unbound antigen-binding molecule. The ability of an antigen-binding molecule to bind to immune cell surface molecule-expressing cells and/or cancer cell antigen-expressing cells can be analysed by methods such as flow cytometry and immunofluorescence microscopy, e.g. as described in the experimental examples of the present application.

In some embodiments, antigen-binding molecules described herein remains on the cell surface of the cells to which they bind. In some embodiments, antigen-binding molecules described herein may not be completely internalised by the cells to which they bind (i.e. internalisation may be incomplete). In some embodiments, the antigen-binding molecule described herein displays less than 100% internalisation, e.g. one of ≤99%, ≤98%, ≤97%, ≤96%, ≤95%, ≤94%, ≤93%, ≤92%, ≤91%, ≤90%, ≤85%, ≤80%, ≤75%, ≤70%, ≤65%, ≤60%, ≤55%, ≤50%, ≤45%, ≤40%, ≤35%, ≤30%, ≤25%, ≤20%, ≤15%, ≤10%, ≤5% by cells to which the antigen-binding molecule binds.

For example, where the cancer cell antigen is mortalin, the antigen-binding molecule may not be completely internalised by mortalin-expressing cells (e.g. cells expressing mortalin at the cell surface, e.g. PANC-1, U251 and/or HCT116 cells).

Internalisation of an antigen-binding molecule by cells can be analysed e.g. by contacting live cells with the antigen-binding molecule, and detecting the antigen-binding molecule after sufficient period of time for internalisation. Internalisation can be determined by detection of the localisation of the antigen-binding molecule. Where the antigen-binding molecule remains on the surface of the cell (e.g. is detected on the cell surface, and/or is not detected inside the cell), the antigen-binding molecule is determined not to have been internalised. Where the antigen-binding molecule is detected inside the cell (e.g. localised to the cytoplasm or a cellular organelle), the antigen-binding molecule is determined to have been internalised.

Internalisation of an antigen-binding molecule can be analysed by methods such as flow cytometry, or immunofluorescence microscopy, e.g. as described in the experimental examples of the present application. Assays for antigen-binding molecule internalisation include those described e.g. in Naith et al., J Immunol Methods (2016) 431:11-21 and Liao-Chan et al., PLoS One (2015) 10(4): e0124708. Percentages of cell-surface bound and/or internalised antigen-binding molecule can be determined.

Advantageously, antigen-binding molecules which remain on the cell surface of the cells to which they bind provide for simultaneous binding of cells (e.g. simultaneous binding of cells expressing the cancer cell antigen, and of cells expressing the immune cell surface molecule).

In some embodiments, the antigen-binding molecule described herein is capable of increasing killing of cells expressing the cancer cell antigen by cells expressing the immune cell surface molecule.

For example, in embodiments wherein the cancer cell surface molecule is mortalin and wherein the immune cell surface molecule is a CD3-TCR complex polypeptide (e.g. CD3E), the antigen-binding molecule may be capable of increasing/enhancing killing of cells expressing mortalin by T cells.

The cancer cell antigen-expressing cells preferably express the cancer cell antigen at the cell surface. That is, in some embodiments ‘cancer cell antigen-expressing cells’ are cells expressing the cancer cell antigen at the cell surface. The cancer cell antigen-expressing cells may be cancer cells. For example, in embodiments wherein the cancer cell surface molecule is mortalin, mortalin-expressing cells may be pancreatic cancer cells, glioblastoma cells and/or colorectal carcinoma cells (e.g. PANC-1, U251 and/or HCT116 cells).

The cells expressing the immune cell surface molecule may be T cells. T cells may be effector T cells. The T cells may be CD8+ T cells, e.g. cytotoxic T lymphocytes (CTLs).

Cell killing may be through induction of apoptosis, cytotoxicity and/or cell lysis (cytolysis) of the cancer cell antigen-expressing cells. Cell killing may involve expression by the T cell of one of more of perforin, granzyme A, granzyme B, FAS ligand, and interferon-γ.

The ability of a given antigen-binding molecule to increase/enhance killing of cells expressing the cancer cell antigen by cells expressing the immune cell surface molecule can be analysed e.g. using an in vitro cell killing assay. Such assays may involve in vitro culture of cancer cell antigen-expressing cells in the presence of the antigen-binding molecule and immune cell surface molecule-expressing cells, and measuring a marker of cell killing after a period of time. Such assays may use isolated T cell populations (e.g. CD8+ T cells) or peripheral blood mononuclear cells. Examples of in vitro cell killing assays include release assays such as the ⁵¹Cr release assay, the lactate dehydrogenase (LDH) release assay (used in the experimental examples of the present application), the 3-(4,5-dimethylthiazol-2-yl)-2,5-diphenyl tetrazolium bromide (MTT) release assay, and the calcein-acetoxymethyl (calcein-AM) release assay. These assays measure cell killing based on the detection of factors released from lysed cells. Release assays and other cell killing assays are described in Zaritskaya et al., Expert Rev Vaccines (2010) 9(6): 601-616.

In some embodiments, the antigen-binding molecule described herein results in a level of cell killing in an in vitro cell killing assay which is more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times, ≥10 times, ≥20 times, ≥30 times, ≥40 times, ≥50 times, ≥60 times, ≥70 times, ≥80 times, ≥90 times, ≥100 times the level of cell killing in the absence of the antigen-binding molecule, or in the presence of an appropriate control antigen-binding molecule (e.g. monospecific antigen-binding molecule having the same specificity as the antigen-binding molecule for one of the targets), in a comparable assay.

In some embodiments, the antigen-binding molecule described herein is capable of inhibiting the development or progression of a cancer, e.g. as determined in an in vitro or in vivo assay in a model for the development and/or progression of the cancer. The cancer may be a cancer expressing the cancer cell antigen, or may comprise cells expressing the cancer cell antigen.

Inhibition of the development of a cancer may be inferred by observation of a delayed or prevented onset of, and/or reduced severity of, symptoms of the cancer in response to treatment with the antigen-binding molecule. Inhibition of the progression of a cancer may be inferred by observation of delayed, prevented and/or reduced invasion and/or metastasis in response to treatment with the antigen-binding molecule.

In some embodiments, the antigen-binding molecule described herein is capable of inhibiting the development or progression of a cancer to less than 100%, e.g. one of 99% or less, 95% or less, 90% or less, 85% or less, 75% or less, 70% or less, 65% or less, 60% or less, 55% or less, 50% or less, 45% or less, 40% or less, 35% or less, 30% or less, 25% or less, 20% or less, 15% or less, 10% or less, 5% or less, or 1% or less of the development/progression of the cancer in the absence of treatment with the antigen-binding molecule (or in the presence of treatment with an appropriate control). In some embodiments, the antigen-binding molecule described herein is capable of inhibiting the development or progression of a cancer to less than 1 times, e.g. one of ≤0.99 times, ≤0.95 times, ≤50.9 times, ≤0.85 times, ≤0.8 times, ≤0.85 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of development/progression of the cancer in the absence of treatment with the antigen-binding molecule (or in the presence of treatment with an appropriate control).

Compositions

Also provided are compositions comprising the antigen-binding molecule described herein.

Antigen-binding molecules according to described herein may be formulated as pharmaceutical compositions or medicaments for clinical use and may comprise a pharmaceutically acceptable carrier, diluent, excipient or adjuvant. The composition may be formulated for topical, parenteral, systemic, intracavitary, intravenous, intra-arterial, intramuscular, intrathecal, intraocular, intraconjunctival, intratumoral, subcutaneous, intradermal, intrathecal, oral or transdermal routes of administration which may include injection or infusion. Suitable formulations may comprise the antigen-binding molecule in a sterile or isotonic medium. Medicaments and pharmaceutical compositions may be formulated in fluid, including gel, form. Fluid formulations may be formulated for administration by injection or infusion (e.g. via catheter) to a selected region of the human or animal body.

Antigen-binding molecules described herein may be formulated as pharmaceutical compositions for clinical use and may comprise a pharmaceutically acceptable carrier, diluent, excipient or adjuvant.

Methods are also provided herein for the production of pharmaceutically useful compositions, such methods of production may comprise one or more steps selected from: isolating as antigen-binding molecule as described herein; and/or mixing an antigen-binding molecule as described herein with a pharmaceutically acceptable carrier, adjuvant, excipient or diluent.

For example, a further aspect provided herein relates to a method of formulating or producing a medicament or pharmaceutical composition for use in the treatment of a cancer, the method comprising formulating a pharmaceutical composition or medicament by mixing an antigen-binding molecule as described herein with a pharmaceutically acceptable carrier, adjuvant, excipient or diluent.

Therapeutic and Prophylactic Applications

The antigen-binding molecules and pharmaceutical compositions described herein find use in therapeutic and prophylactic methods.

Provided herein is an antigen-binding molecule or pharmaceutical composition described herein for use in a method of medical treatment or prophylaxis. Also provided herein is the use of an antigen-binding molecule or pharmaceutical composition described herein in the manufacture of a medicament for treating or preventing a disease or condition. Further provided herein is a method of treating or preventing a disease or condition, comprising administering to a subject a therapeutically or prophylactically effective amount of an antigen-binding molecule or pharmaceutical composition described herein.

‘Treatment’ may, for example, be reduction in the development or progression of a disease/condition, alleviation of the symptoms of a disease/condition or reduction in the pathology of a disease/condition. Treatment or alleviation of a disease/condition may be effective to prevent progression of the disease/condition, e.g. to prevent worsening of the condition or to slow the rate of development. In some embodiments treatment or alleviation may lead to an improvement in the disease/condition, e.g. a reduction in the symptoms of the disease/condition or reduction in some other correlate of the severity/activity of the disease/condition. Prevention of a disease/condition may refer to prevention of a worsening of the condition or prevention of the development of the disease/condition, e.g. preventing an early stage disease/condition developing to a later, chronic, stage.

In particular, the antigen-binding molecules and pharmaceutical compositions described herein find use to treat or prevent cancers.

The cancer to be treated/prevented may be any unwanted cell proliferation (or any disease manifesting itself by unwanted cell proliferation), neoplasm or tumor. The cancer may be benign or malignant and may be primary or secondary (metastatic). A neoplasm or tumor may be any abnormal growth or proliferation of cells and may be located in any tissue. The cancer may be of tissues/cells derived from e.g. the adrenal gland, adrenal medulla, anus, appendix, bladder, blood, bone, bone marrow, brain, breast, cecum, central nervous system (including or excluding the brain) cerebellum, cervix, colon, duodenum, endometrium, epithelial cells (e.g. renal epithelia), gallbladder, oesophagus, glial cells, heart, ileum, jejunum, kidney, lacrimal glad, larynx, liver, lung, lymph, lymph node, lymphoblast, maxilla, mediastinum, mesentery, myometrium, nasopharynx, omentum, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, white blood cells.

The cancer to be treated/prevented may be any kind of cancer, including any one of an acute lymphoblastic leukemia (ALL), acute myeloid leukemia (AML), adrenocortical carcinoma, AIDS-related cancer (e.g. Kaposi sarcoma, AIDS-related lymphoma, primary CNS lymphoma), anal cancer, appendix cancer, astrocytoma, basal cell carcinoma of the skin, bile duct cancer (e.g. cholangiocarcinoma), bladder cancer, bone cancer (e.g. Ewing sarcoma, osteosarcoma, malignant fibrous histiocytoma), brain tumor, breast cancer, bronchial tumor, Burkitt lymphoma, carcinoid tumor, carcinoma of unknown primary, cardiac tumor, central nervous system cancer (e.g. atypical teratoid/rhabdoid tumor, embryonal tumor, germ cell tumor, primary CNS lymphoma), cervical cancer, chordoma, chronic lymphocytic leukemia (CLL), chronic myelogenous leukemia (CML), chronic myeloproliferative neoplasm, colorectal cancer, craniopharyngioma, cutaneous T-cell lymphoma (e.g. mycosis fungoides, Sézary syndrome), ductal carcinoma in situ (DCIS), endometrial cancer (uterine cancer), ependymoma, esophageal cancer, esthesioneuroblastoma, extracranial germ cell tumor, extragonadal germ cell tumor, eye cancer (e.g. intraocular melanoma, retinoblastoma) fallopian tube cancer, malignant fibrous histiocytoma of bone, gallbladder cancer, gastric (stomach) cancer, gastrointestinal carcinoid tumor, gastrointestinal stromal tumor (GIST), ovarian germ cell tumor, testicular cancer, gestational trophoblastic disease, hairy cell leukemia, head and neck cancer, heart tumor, hepatocellular (liver) cancer, histiocytosis, Langerhans cell, Hodgkin lymphoma, hypopharyngeal cancer, islet cell tumor (pancreatic neuroendocrine tumor), kidney (renal cell) cancer, laryngeal cancer, papillomatosis, leukemia, lip and oral cavity cancer, lung cancer (non-small cell lung cancer (NSCLC) and small cell lung cancer (SCLC)) lymphoma, male breast cancer, melanoma, Merkel cell carcinoma, mesothelioma, metastatic cancer, metastatic squamous neck cancer with occult primary, midline tract carcinoma involving NUT gene, mouth cancer, multiple endocrine neoplasia syndromes, multiple myeloma/plasma cell neoplasms, mycosis fungoides, myelodysplastic syndrome, myelodysplastic/myeloproliferative neoplasm, myelogenous leukemia, chronic myeloid leukemia, acute myeloid leukemia (AML), nasal cavity and paranasal sinus cancer, nasopharyngeal cancer, neuroblastoma, non-Hodgkin lymphoma, oral cancer, lip and oral cavity cancer, oropharyngeal cancer, osteosarcoma, ovarian cancer, pancreatic cancer, papillomatosis, paraganglioma, paranasal sinus cancer, nasal cavity cancer, parathyroid cancer, penile cancer, pharyngeal cancer, pheochromocytoma, pituitary tumor, plasma cell neoplasm/multiple myeloma, pleuropulmonary blastoma, pregnancy and breast cancer, primary peritoneal cancer, prostate cancer, rectal cancer, recurrent cancer, retinoblastoma, rhabdomyosarcoma, salivary gland cancer, vascular tumor, uterine sarcoma, skin cancer, small intestine cancer, squamous cell carcinoma of the skin, T-cell lymphoma, throat cancer, thymoma, thymic carcinoma, thyroid cancer, transitional cell cancer of the renal pelvis and ureter, urethral cancer, vaginal cancer, vulvar cancer or Wilms tumor.

In some embodiments, the cancer to be treated/prevented comprises cells expressing a cancer cell antigen, e.g. a cancer cell antigen as described herein (e.g. mortalin). In some embodiments, the cells express the cancer cell antigen at the cell surface.

In some embodiments, the cancer to be treated comprises cells expressing a cancer cell antigen for which the antigen-binding molecule is specific. In some embodiments, the antigen-binding molecule comprises a cancer cell antigen-binding domain, and the cancer to be treated/prevented comprises cells expressing the cancer cell antigen, e.g. cells expressing cancer cell antigen at the cell surface.

In some embodiments, the cancer to be treated/prevented is a cancer which is positive for the cancer cell antigen. For example, where the cancer cell antigen is mortalin, the cancer may be a “mortalin-positive cancer”. In some embodiments, the cancer over-expresses the cancer cell antigen. Overexpression of a cancer cell antigen can be determined by detection of a level of expression of the cancer cell antigen which is greater than the level of expression by equivalent non-cancerous cells/non-tumor tissue.

Cancer cell antigen expression may be determined by any suitable means. Expression may be gene expression or protein expression. Gene expression can be determined e.g. by detection of mRNA encoding the cancer cell antigen, for example by quantitative real-time PCR (qRT-PCR). Protein expression can be determined e.g. by detection of the cancer cell antigen, for example by antibody-based methods, for example by western blot, immunohistochemistry, immunocytochemistry, flow cytometry, or ELISA.

In some embodiments, a patient may be selected for treatment described herein based on the detection of a cancer expressing the cancer cell antigen, or overexpressing the cancer cell antigen, e.g. in a sample obtained from the subject.

Mortalin has been implicated in a variety of cancers, and is upregulated in the tumors derived from a range of tissues (Wadhwa et al., (2006) Int J Cancer 118(12):2973-80). Mortalin is over-expressed, and correlates with poor survival in colorectal adenocarcinomas, is upregulated in hepatocellular carcinoma and associated with metastasis, and is also implicated in gastric cancer and breast cancer (Dundas et al., J Pathol (2005) 205: 74-81; Rozenberg et al., Int J Cancer (2013) 133:514-518; Chen et al., Anat Rec (Hoboken) (2011) 294:1344-1351; Yi et al., Mol Cell Proteomics (2008) 7(2):315-25; Ando et al., Gastric Cancer (2014) 17(2):255-62; Jin et al. J Exp Clin Cancer Res (2016) 35:42). Surface expression of mortalin has been proposed to contribute to resistance to complement-dependent cytotoxicity (CDC) of cancer cells by facilitating elimination of the complement membrane attack complex (MAC) from the cell surface through exo-vesiculation (Pilzer et al. Int Immunol (2005) 17:1239-48).

In some embodiments the cancer is a cancer expressing mortalin, e.g. a cancer expressing mortalin at the cell surface (i.e. is expressed in or at the cell membrane). In some embodiments, the cancer over-expresses mortalin. Overexpression of mortalin can be determined by detection of a level of expression of mortalin which is greater than the level of expression of mortalin by equivalent non-cancerous cells/non-tumor tissue.

In some embodiments, the cancer to be treated/prevented is pancreatic cancer (e.g. pancreatic carcinoma), brain cancer (e.g. glioblastoma), colorectal cancer (e.g. colorectal carcinoma), liver cancer (e.g. hepatocellular carcinoma), breast cancer or gastric cancer.

In some embodiments, the cancer to be treated/prevented is pancreatic cancer (e.g. pancreatic carcinoma), brain cancer (e.g. glioblastoma), colorectal cancer (e.g. colorectal carcinoma), liver cancer (e.g. hepatocellular carcinoma), breast cancer or gastric cancer which comprises cells expressing the cancer cell antigen at the cell surface. In some embodiments, the cancer to be treated/prevented is pancreatic cancer (e.g. pancreatic carcinoma), brain cancer (e.g. glioblastoma), colorectal cancer (e.g. colorectal carcinoma), liver cancer (e.g. hepatocellular carcinoma) or gastric cancer comprising cells which over-express the cancer cell antigen.

The treatment may be aimed at reducing the number of cells of the cancer, and/or reducing the size of a tumour.

Administration of the antigen-binding molecules and compositions described herein may delay or prevent the onset of symptoms of the cancer. Administration of the antigen-binding molecules and compositions described herein may reduce the severity of symptoms of the cancer. Administration of the antigen-binding molecules and compositions described herein may delay or prevent the onset of invasion and/or metastasis. Administration of the antigen-binding molecules and compositions described herein reduce invasion and/or metastasis.

Methods of medical treatment may also involve in vivo, ex vivo, and adoptive immunotherapies, including those using autologous and/or heterologous cells or immortalised cell lines.

Administration of an antigen-binding molecule or composition described herein is preferably in a “therapeutically effective” or “prophylactically effective” amount, this being sufficient to show benefit to the subject. The actual amount administered, and rate and time-course of administration, will depend on the nature and severity of the disease or disorder. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Administration may be alone or in combination with other treatments, either simultaneously or sequentially dependent upon the condition to be treated. The antigen-binding molecule or composition described herein and a therapeutic agent may be administered simultaneously or sequentially.

In some embodiments, the methods comprise additional therapeutic or prophylactic intervention, e.g. for the treatment/prevention of a cancer. In some embodiments, the therapeutic or prophylactic intervention is selected from chemotherapy, immunotherapy, radiotherapy, surgery, vaccination and/or hormone therapy.

Simultaneous administration refers to administration of the antigen-binding molecule, nucleic acid, vector, cell or composition and therapeutic agent together, for example as a pharmaceutical composition containing both agents (combined preparation), or immediately after each other and optionally via the same route of administration, e.g. to the same artery, vein or other blood vessel. Sequential administration refers to administration of one of the antigen-binding molecule/composition or therapeutic agent followed after a given time interval by separate administration of the other agent. It is not required that the two agents are administered by the same route, although this is the case in some embodiments. The time interval may be any time interval.

Chemotherapy and radiotherapy respectively refer to treatment of a cancer with a drug or with ionising radiation (e.g. radiotherapy using X-rays or γ-rays). The drug may be a chemical entity, e.g. small molecule pharmaceutical, antibiotic, DNA intercalator, protein inhibitor (e.g. kinase inhibitor), or a biological agent, e.g. antibody, antibody fragment, aptamer, nucleic acid (e.g. DNA, RNA), peptide, polypeptide, or protein. The drug may be formulated as a pharmaceutical composition or medicament.

The formulation may comprise one or more drugs (e.g. one or more active agents) together with one or more pharmaceutically acceptable diluents, excipients or carriers.

A treatment may involve administration of more than one drug. A drug may be administered alone or in combination with other treatments, either simultaneously or sequentially dependent upon the condition to be treated. For example, the chemotherapy may be a co-therapy involving administration of two drugs, one or more of which may be intended to treat the cancer.

The chemotherapy may be administered by one or more routes of administration, e.g. parenteral, intravenous injection, oral, subcutaneous, intradermal or intratumoral.

The chemotherapy may be administered according to a treatment regime. The treatment regime may be a pre-determined timetable, plan, scheme or schedule of chemotherapy administration which may be prepared by a physician or medical practitioner and may be tailored to suit the patient requiring treatment.

The treatment regime may indicate one or more of: the type of chemotherapy to administer to the patient; the dose of each drug or radiation; the time interval between administrations; the length of each treatment; the number and nature of any treatment holidays, if any etc. For a co-therapy a single treatment regime may be provided which indicates how each drug is to be administered.

Chemotherapeutic drugs and biologics may be selected from: alkylating agents such as cisplatin, carboplatin, mechlorethamine, cyclophosphamide, chlorambucil, ifosfamide; purine or pyrimidine anti-metabolites such as azathiopurine or mercaptopurine; alkaloids and terpenoids, such as vinca alkaloids (e.g. vincristine, vinblastine, vinorelbine, vindesine), podophyllotoxin, etoposide, teniposide, taxanes such as paclitaxel (Taxol™), docetaxel; topoisomerase inhibitors such as the type I topoisomerase inhibitors camptothecins irinotecan and topotecan, or the type II topoisomerase inhibitors amsacrine, etoposide, etoposide phosphate, teniposide; antitumor antibiotics (e.g. anthracyline antibiotics) such as dactinomycin, doxorubicin (Adriamycin™), epirubicin, bleomycin, rapamycin; antibody based agents, such as anti-PD-1 antibodies, anti-PD-L1 antibodies, anti-TIM-3 antibodies, anti-CTLA-4, anti-4-1BB, anti-GITR, anti-CD27, anti-BLTA, anti-OX43, anti-VEGF, anti-TNFα, anti-IL-2, antiGpIIb/IIIa, anti-CD-52, anti-CD20, anti-RSV, anti-HER2/neu(erbB2), anti-TNF receptor, anti-EGFR antibodies, monoclonal antibodies or antibody fragments, examples include: cetuximab, panitumumab, infliximab, basiliximab, bevacizumab (Avastin®), abciximab, daclizumab, gemtuzumab, alemtuzumab, rituximab (Mabthera®), palivizumab, trastuzumab, etanercept, adalimumab, nimotuzumab; EGFR inihibitors such as erlotinib, cetuximab and gefitinib; anti-angiogenic agents such as bevacizumab (Avastin®); cancer vaccines such as Sipuleucel-T (Provenge®).

Further chemotherapeutic drugs may be selected from: 13-cis-Retinoic Acid, 2-Chlorodeoxyadenosine, 5-Azacitidine 5-Fluorouracil, 6-Mercaptopurine, 6-Thioguanine, Abraxane, Accutane®, Actinomycin-D Adriamycin®, Adrucil®, Afinitor®, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Aranesp®, Aredia®, Arimidex®, Aromasin®, Arranon®, Arsenic Trioxide, Asparaginase, ATRA Avastin®, Azacitidine, BCG, BCNU, Bendamustine, Bevacizumab, Bexarotene, BEXXAR®, Bicalutamide, BiCNU, Blenoxane®, Bleomycin, Bortezomib, Busulfan, Busulfex®, Calcium Leucovorin, Campath®, Camptosar®, Camptothecin-11, Capecitabine, Carac™, Carboplatin, Carmustine, Casodex®, CC-5013, CCI-779, CCNU, CDDP, CeeNU, Cerubidine®, Cetuximab, Chlorambucil, Cisplatin, Citrovorum Factor, Cladribine, Cortisone, Cosmegen®, CPT-11, Cyclophosphamide, Cytadren®, Cytarabine Cytosar-U®, Cytoxan®, Dacogen, Dactinomycin, Darbepoetin Alfa, Dasatinib, Daunomycin, Daunorubicin, Daunorubicin Hydrochloride, Daunorubicin Liposomal, DaunoXome®, Decadron, Decitabine, Delta-Cortef®, Deltasone®, Denileukin, Diftitox, DepoCyt™, Dexamethasone, Dexamethasone Acetate, Dexamethasone Sodium Phosphate, Dexasone, Dexrazoxane, DHAD, DIC, Diodex, Docetaxel, Doxil®, Doxorubicin, Doxorubicin Liposomal, Droxia™, DTIC, DTIC-Dome®, Duralone®, Eligard™, Ellence™, Eloxatin™, Elspar®, Emcyt®, Epirubicin, Epoetin Alfa, Erbitux, Erlotinib, Erwinia L-asparaginase, Estramustine, Ethyol Etopophos®, Etoposide, Etoposide Phosphate, Eulexin®, Everolimus, Evista®, Exemestane, Faslodex®, Femara®, Filgrastim, Floxuridine, Fludara®, Fludarabine, Fluoroplex®, Fluorouracil, Fluoxymesterone, Flutamide, Folinic Acid, FUDR®, Fulvestrant, Gefitinib, Gemcitabine, Gemtuzumab ozogamicin, Gleevec™, Gliadel® Wafer, Goserelin, Granulocyte—Colony Stimulating Factor, Granulocyte Macrophage Colony Stimulating Factor, Herceptin®, Hexadrol, Hexalen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab Tiuxetan, Idamycin®, Idarubicin, Ifex®, IFN-alpha, Ifosfamide, IL-11, IL-2, Imatinib mesylate, Imidazole Carboxamide, Interferon alfa, Interferon Alfa-2b (PEG Conjugate), Interleukin-2, Interleukin-11, Intron A® (interferon alfa-2b), Iressa®, Irinotecan, Isotretinoin, Ixabepilone, Ixempra™, Kidrolase, Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred®, Lomustine, L-PAM, L-Sarcolysin, Lupron®, Lupron Depot®, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, Mustine, Mutamycin®, Myleran®, Mylocel™, Mylotarg®, Navelbine®, Nelarabine, Neosar, Neulasta™, Neumega®, Neupogen®, Nexavar, Nilandron®, Nilutamide, Nipent®, Nitrogen Mustard, Novaldex®, Novantrone®, Octreotide, Octreotide acetate, Oncospar®, Oncovin®, Ontak®, Onxal™, Oprevelkin, Orapred®, Orasone®, Oxaliplatin, Paclitaxel, Paclitaxel Protein-bound, Pamidronate, Panitumumab, Panretin®, Paraplatin®, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant Purinethol®, Raloxifene, Revlimid®, Rheumatrex®, Rituxan®, Rituximab, Roferon-A® (Interferon Alfa-2a), Rubex®, Rubidomycin hydrochloride, Sandostatin® Sandostatin LAR®, Sargramostim, Solu-Cortef®, Solu-Medrol®, Sorafenib, SPRYCEL™, STI-571, Streptozocin, SU11248, Sunitinib, Sutent®, Tamoxifen, Tarceva®, Targretin®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Torisel®, Tositumomab, Trastuzumab, Treanda®, Tretinoin, Trexall™, Trisenox®, TSPA, TYKERB®, VCR, Vectibix™, Velban®, Velcade®, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, VP-16, Vumon®, Xeloda®, Zanosar®, Zevalin™, Zinecard®, Zoladex®, Zoledronic acid, Zolinza, Zometa®.

Multiple doses of the antigen-binding molecule or composition may be provided. One or more, or each, of the doses may be accompanied by simultaneous or sequential administration of another therapeutic agent.

Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days).

Methods of Detection

Antigen-binding molecules described herein may be used in methods that involve the antigen-binding molecule to the cancer cell antigen and/or the immune cell surface molecule to which the antigen-binding molecule is capable of binding. Such methods may involve detection of the bound complex of the antigen-binding molecule and the cancer cell antigen and/or the immune cell surface molecule.

As such, a method is provided, the method comprising contacting a sample containing, or suspected to contain, the cancer cell antigen and/or the immune cell surface molecule, and detecting the formation of a complex of the antigen-binding molecule and the cancer cell antigen and/or the immune cell surface molecule. Also provided is a method comprising contacting a sample containing, or suspected to contain, a cell expressing the cancer cell antigen and/or a cell expressing the immune cell surface molecule, and detecting the formation of a complex of the antigen-binding molecule and a cell expressing the cancer cell antigen and/or a cell expressing the immune cell surface molecule.

Suitable method formats are well known in the art, including immunoassays such as sandwich assays, e.g. ELISA. The methods may involve labelling the antigen-binding molecule, or target(s), or both, with a detectable moiety, e.g. a detectable moiety as described hereinabove. In some embodiment the detectable moiety is a fluorescent label, a luminescent label, an immuno-detectable label or a radio-label. In some embodiments, the detectable moiety may be selected from: a radio-nucleotide, positron-emitting radionuclide (e.g. for positron emission tomography (PET)), MRI contrast agent or fluorescent label. Analysis in vitro or in vivo may involve analysis by positron emission tomography (PET), magnetic resonance imaging (MRI), or fluorescence imaging, e.g. by detection of appropriately labelled species.

Methods of this kind may provide the basis of methods for the diagnostic and/or prognostic evaluation of a disease or condition, e.g. a cancer. Such methods may be performed in vitro on a patient sample, or following processing of a patient sample. Once the sample is collected, the patient is not required to be present for the in vitro method to be performed, and therefore the method may be one which is not practised on the human or animal body.

Detection in a sample may be used for the purpose of diagnosis of a disease or condition (e.g. a cancer), predisposition to a disease or condition, or for providing a prognosis (prognosticating) for a disease or condition. The diagnosis or prognosis may relate to an existing (previously diagnosed) disease or condition.

Such methods may involve detecting or quantifying one or more of: the cancer cell antigen, cells expressing the cancer cell antigen, the immune cell surface molecule, or cells expressing the immune cell surface molecule, e.g. in a patient sample. Where the method comprises quantifying the relevant factor, the method may further comprise comparing the determined amount against a standard or reference value as part of the diagnostic or prognostic evaluation. Other diagnostic/prognostic tests may be used in conjunction with those described herein to enhance the accuracy of the diagnosis or prognosis or to confirm a result obtained by using the tests described herein.

A sample may be taken from any tissue or bodily fluid. The sample may comprise or may be derived from: a quantity of blood; a quantity of serum derived from the individual's blood which may comprise the fluid portion of the blood obtained after removal of the fibrin clot and blood cells; a tissue sample or biopsy; pleural fluid; cerebrospinal fluid (CSF); or cells isolated from said individual. In some embodiments, the sample may be obtained or derived from a tissue or tissues which are affected by the disease/condition (e.g. tissue or tissues in which symptoms of the disease manifest, or which are involved in the pathogenesis of the disease/condition).

Methods described herein may preferably performed in vitro. The term “in vitro” is intended to encompass experiments with cells in culture whereas the term “in vivo” is intended to encompass experiments with intact multi-cellular organisms.

Subjects

The subject to be treated described herein may be any animal or human. The subject is preferably mammalian, more preferably human. The subject may be a non-human mammal, but is more preferably human. The subject may be male or female. The subject may be a patient. A subject may have been diagnosed with a disease or condition requiring treatment (e.g. a cancer), may be suspected of having such a disease/condition, or may be at risk of developing/contracting such a disease/condition.

Kits

In some aspects, a kit of parts is provided. In some embodiments the kit may have at least one container having a predetermined quantity of an antigen-binding molecule or composition described herein.

The kit may provide the antigen-binding molecule or composition together with instructions for administration to a patient in order to treat a specified disease/condition. The antigen-binding molecule or composition may be formulated so as to be suitable for injection or infusion to a tumor or to the blood.

In some embodiments, the kit may comprise materials for producing antigen-binding molecule or composition described herein.

In some embodiments the kit may further comprise at least one container having a predetermined quantity of another therapeutic agent (e.g. anti-infective agent or chemotherapy agent). In such embodiments, the kit may also comprise a second medicament or pharmaceutical composition such that the two medicaments or pharmaceutical compositions may be administered simultaneously or separately such that they provide a combined treatment for the specific disease or condition. The therapeutic agent may also be formulated so as to be suitable for injection or infusion to a tumor or to the blood.

Sequence Identity

Pairwise and multiple sequence alignment for the purposes of determining percent identity between two or more amino acid or nucleic acid sequences can be achieved in various ways known to a person of skill in the art, for instance, using publicly available computer software such as ClustalOmega (Soding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6(298)) and MAFFT (Katoh and Standley 2013, Molecular Biology and Evolution, 30(4) 772-780 software. When using such software, the default parameters, e.g. for gap penalty and extension penalty, are preferably used.

Sequences

| SEQ | | |
|---|---|---|
| ID NO: | DESCRIPTION | SEQUENCE |
| 1 | tP19 (mortalin- | CUCAAUGGCGAAUGCCCGCCUAAUAGGG |
| | binding) | |
| 2 | P19 (mortalin- | GGGAGACAAGAAUAAACGCUCAAUGGCGAAUGCCCGCCUAAUAGGGCGUUAUG |
| | binding) | ACUUGUUGAGUUCGACAGGAGGCUCACAACAGGC |
| 3 | P1 (mortalin- | GGGAGACAAGAAUAAACGCUCAAUGCGCUGAAUGCCCAGCCGUGAAAGCGUCG |
| | binding) | AUUUCCAUCCUUCGACAGGAGGCUCACAACAGGC |
| 4 | P15 (vimentin- | GGGAGACAAGAAUAAACGCUCAAAGUUGCGGCCCAACCGUUUAAUUCAGAAUAG |
| | binding) | UGUGAUGCCUUCGACAGGAGGCUCACAACAGGC |
| 5 | P11 (HSP90- | GGGAGACAAGAAUAAACGCUCAAAUGAUUGCCCAUUCGGUUAUGCUUGCGCUU |
| | binding) | CCUAAAGAGCUUCGACAGGAGGCUCACAACAGGC |
| 6 | P7 (HSP90- | GGGAGACAAGAAUAAACGCUCAAGGCCAUGUUGAAUGCCCAACUAAGCUUUGAG |
| | binding) | CUUUGGAGCUUCGACAGGAGGCUCACAACAGGC |
| 7 | P6 (HSP90- | GGGAGACAAGAAUAAACGCUCAACAAUGGAGCGUUAAACGUGAGCCAUUCGACA |
| | binding) | GGAGGCUCACAACAGGC |
| 8 | P19, P1, P7 | GAAUGCCC |
| | consensus | |
| 9 | TR14 (TfR- | GGGAGACAAGAAUAAACGCUCAAUGCGUUCACGUUUAUUCACAUUUUUGAAUUG |
| | binding) | AGCAUGAGCUUCGACAGGAGGCUCACAACAGGC |
| 10 | TR18 (TfR- | GGGAGACAAGAAUAAACGCUCAAUGCGUUUACGUUUAUUCACAUUUUUGAAUUG |
| | binding) | AGCAUGAGCUUCGACAGGAGGCUCACAACAGGC |
| 11 | Truncated TR14 | GGGGCUCAAUGCGUUCACGUUUAUUCACAUUUUUGAAUUGAGC |
| | (TfR-binding) | |
| | 43mer | |
| 12 | PDR3 (PDGFR- | GGGAGAGCGGAAGCGUGCUGGGCCUGCUCUUUAAUAAACCCACUUUCGAACAU |
| | a-binding) | CAGCGUAUGUCCAUAACCCAGAGGUGAUGGAUCCCCC |
| 13 | PDR9 (PDGFR- | GGGAGAGCGGAAGCGUGCUGGGCCUAUUGCAUCUUUCUGUUAUUUCCGAAUCC |
| | a-binding) | GUCCCGACUGUCAUAACCCAGAGGUGAUGGAUCCCCC |
| 14 | G3 (CCR5- | GGGAGGACGAUGCGGGCCUUCGUUUGUUUCGUCCACAGACGACUCGCCCGA |
| | binding) | |
| 15 | C3e2 (CD3s- | GGAGACAAGAAUAAACGCUCAAAUAGAAGCAGCAUCUUCCAAAUCAGUUUGUGU |
| | binding) | GUCCUCUAUUCGACAGGAGGCUCACAACAGGC |
| 16 | C3e3 (CD3ε- | GGGAGACAAGAAUAAACGCUCAAAUGCCUGUAGUUCGUAGCGAUUUAACUGCG |
| | binding) | UCAGUGAGGCUUCGACAGGAGGCUCACAACAGGC |
| 17 | 36-7 VL | DVVMTQTPLTLSVTIGQPASISCKSSQSLLDSDGKTYLNWLLQRPGQSPKRLIYLVSKL |
| | | DSGVPDRFTGSGSGTDFTLKISRVEAEDLGVYYCWQGTHFPRTFGGGTKLEIKR |
| 18 | 36-7 LC-CDR1 | KSSQSLLDSDGKTYLN |
| 19 | 36-7 LC-CDR2 | LVSKLDS |
| 20 | 36-7 LC-CDR3 | WQGTHFPRT |
| 21 | 36-7 VH | QVQLQQPGAELVKPGASVKLSCKASGYTFTSYWMHWVKQRPGQGLEWIGEIDPSDS |
| | | YTKYNQKFKGKATLTVDKSSSTAYMQLSSLTSEDSAVYYCARGDYWGQGTTLTVSS |
| 22 | 36-7 HC-CDR1 | SYWMH |
| 23 | 36-7 HC-CDR2 | EIDPSDSYTKYNQKFKG |
| 24 | 36-7 HC-CDR3 | GDY |
| 25 | MT-103 VL | DIQLTQSPAIMSASPGEKVTMTCRASSSVSYMNVVYQQKSGTSPKRWIYDTSKVASGV |
| | | PYRFSGSGSGTSYSLTISSMEAEDAATYYCQQWSSNPLTFGAGTKLELK |
| 26 | MT-103 LC-CDR1 | RASSSVSYMN |
| 27 | MT-103 LC-CDR2 | DTSKVAS |
| 28 | MT-103 LC-CDR3 | QQWSSNPLT |
| 29 | MT-103 VH | DIKLQQSGAELARPGASVKMSCKTSGYTFTRYTMHWVKQRPGQGLEWIGYINPSRG |
| | | YTNYNQKFKDKATLTTDKSSSTAYMQLSSLTSEDSAVYYCARYYDDHYCLDYWGQG |
| | | TTLTVSS |
| 30 | MT-103 HC-CDR1 | RYTMH |
| 31 | MT-103 HC-CDR2 | YINPSRGYTNYNQKFKD |
| 32 | MT-103 HC-CDR3 | YYDDHYCLDY |
| 33 | 36-7 VL | GACGTGGTGATGACCCAGACCCCGCTGACCCTGAGTGTAACGATCGGCCAGCCC |
| | nucleotide | GCCAGTATCAGCTGTAAGAGCAGTCAAAGCCTGCTGGACAGCGACGGCAAGACC |
| | | TACCTGAACTGGCTGCTTCAAAGGCCGGGACAGAGCCCCAAGAGGCTCATCTACC |
| | | TGGTGAGCAAGCTGGACTCAGGCGTGCCCGACAGGTTCACCGGGAGCGGGAGT |
| | | GGCACCGACTTCACCCTGAAGATCAGCAGGGTAGAGGCCGAGGACCTGGGTGTG |
| | | TACTACTGCTGGCAGGGAACCCATTTTCCCAGGACTTTCGGAGGCGGGACCAAAC |
| | | TGGAAATCAAGCGA |
| 34 | 36-7 VH | CAGGTGCAGCTCCAACAGCCAGGTGCCGAGCTGGTAAAACCTGGCGCGTCAGTG |
| | nucleotide | AAGCTGAGTTGTAAAGCAAGCGGCTACACCTTCACCAGCTACTGGATGCACTGGG |
| | | TTAAGCAGAGGCCAGGCCAAGGCCTGGAGTGGATCGGCGAGATCGACCCCAGCG |
| | | ACAGTTACACCAAGTACAACCAGAAGTTCAAGGGCAAGGCAACCCTCACGGTCGA |
| | | TAAGTCAAGCAGCACCGCGTACATGCAACTTAGTAGCCTGACCAGCGAGGATAGC |
| | | GCGGTCTACTATTGCGCCAGAGGCGACTACTGGGGCCAGGGCACCACTTTGACC |
| | | GTGTCTAGT |
| 35 | MT-103 VL | GACATCCAGCTGACCCAGAGCCCTGCCATCATGAGCGCCAGTCCTGGCGAGAAG |
| | nucleotide | GTGACCATGACCTGTAGGGCCTCTAGCTCAGTGAGCTACATGAACTGGTATCAAC |
| | | AGAAAAGCGGCACGAGCCCCAAGAGGTGGATCTACGACACCAGCAAAGTGGCGT |
| | | CAGGCGTACCTTATAGGTTTAGCGGCTCAGGATCAGGCACCAGCTACAGCCTGAC |
| | | CATCAGCTCTATGGAGGCCGAAGACGCCGCCACCTATTATTGCCAGCAGTGGTCT |
| | | AGCAACCCCCTGACCTTCGGAGCCGGCACCAAACTGGAACTGAAG |
| 36 | MT-103 VH | GACATCAAACTTCAGCAGAGCGGCGCTGAGCTGGCTCGACCAGGCGCGAGCGTG |
| | nucleotide | AAGATGAGCTGCAAGACCAGTGGCTATACCTTCACCAGGTACACCATGCACTGGG |
| | | TGAAGCAACGACCTGGACAGGGACTGGAGTGGATCGGCTACATCAACCCCAGCA |
| | | GGGGCTACACCAACTACAACCAGAAGTTCAAGGACAAGGCGACCCTGACGACCG |
| | | ACAAGAGCAGCAGCACCGCCTACATGCAGCTGAGCAGTCTCACCAGCGAGGACA |
| | | GCGCGGTGTACTACTGCGCAAGGTACTACGACGACCACTATTGCCTGGACTACTG |
| | | GGGCCAGGGCACCACGCTGACGGTGAGCTCT |
| 37 | Human mortalin | MISASRAAAARLVGAAASRGPTAARHQDSWNGLSHEAFRLVSRRDYASEAIKGAVVG |
| | (UniProt | IDLGTTNSCVAVMEGKQAKVLENAEGARTTPSVVAFTADGERLVGMPAKRQAVTNPN |
| | P38646) | NTFYATKRLIGRRYDDPEVQKDIKNVPFKIVRASNGDAWVEAHGKLYSPSQIGAFVLM |
| | | KMKETAENYLGHTAKNAVITVPAYFNDSQRQATKDAGQISGLNVLRVINEPTAAALAY |
| | | GLDKSEDKVIAVYDLGGGTFDISILEIQKGVFEVKSTNGDTFLGGEDFDQALLRHIVKEF |
| | | KRETGVDLTKDNMALQRVREAAEKAKCELSSSVQTDINLPYLTMDSSGPKHLNMKLT |
| | | RAQFEGIVTDLIRRTIAPCQKAMQDAEVSKSDIGEVILVGGMTRMPKVQQTVQDLFGR |
| | | APSKAVNPDEAVAIGAAIQGGVLAGDVTDVLLLDVTPLSLGIETLGGVFTKLINRNTTIPT |
| | | KKSQVFSTAADGQTQVEIKVCQGEREMAGDNKLLGQFTLIGIPPAPRGVPQIEVTFDID |
| | | ANGIVHVSAKDKGTGREQQIVIQSSGGLSKDDIENMVKNAEKYAEEDRRKKERVEAVN |
| | | MAEGIIHDTETKMEEFKDQLPADECNKLKEEISKMRELLARKDSETGENIRQAASSLQ |
| | | QASLKLFEMAYKKMASEREGSGSSGTGEQKEDQKEEKQ |
| 38 | Human mortalin | ASEAIKGAVVGIDLGTTNSCVAVMEGKQAKVLENAEGARTTPSVVAFTADGERLVGM |
| | (mature | PAKRQAVTNPNNTFYATKRLIGRRYDDPEVQKDIKNVPFKIVRASNGDAWVEAHGKLY |
| | sequence, | SPSQIGAFVLMKMKETAENYLGHTAKNAVITVPAYFNDSQRQATKDAGQISGLNVLRVI |
| | lacking | NEPTAAALAYGLDKSEDKVIAVYDLGGGTFDISILEIQKGVFEVKSTNGDTFLGGEDFD |
| | mitochondrial | QALLRHIVKEFKRETGVDLTKDNMALQRVREAAEKAKCELSSSVQTDINLPYLTMDSS |
| | signal peptide; | GPKHLNMKLTRAQFEGIVTDLIRRTIAPCQKAMQDAEVSKSDIGEVILVGGMTRMPKV |
| | UniProt P38646 | QQTVQDLFGRAPSKAVNPDEAVAIGAAIQGGVLAGDVTDVLLLDVTPLSLGIETLGGVF |
| | residues 47 to | TKLINRNTTIPTKKSQVFSTAADGQTQVEIKVCQGEREMAGDNKLLGQFTLIGIPPAPR |
| | 679) | GVPQIEVTFDIDANGIVHVSAKDKGTGREQQIVIQSSGGLSKDDIENMVKNAEKYAEED |
| | | RRKKERVEAVNMAEGIIHDTETKMEEFKDQLPADECNKLKEEISKMRELLARKDSETG |
| | | ENIRQAASSLQQASLKLFEMAYKKMASEREGSGSSGTGEQKEDQKEEKQ |
| 39 | Human vimentin | MSTRSVSSSSYRRMFGGPGTASRPSSSRSYVTTSTRTYSLGSALRPSTSRSLYASSP |
| | (UniProt | GGVYATRSSAVRLRSSVPGVRLLQDSVDFSLADAINTEFKNTRTNEKVELQELNDRFA |
| | P08670) | NYIDKVRFLEQQNKILLAELEQLKGQGKSRLGDLYEEEMRELRRQVDQLTNDKARVEV |
| | | ERDNLAEDIMRLREKLQEEMLQREEAENTLQSFRQDVDNASLARLDLERKVESLQEEI |
| | | AFLKKLHEEEIQELQAQIQEQHVQIDVDVSKPDLTAALRDVRQQYESVAAKNLQEAEE |
| | | WYKSKFADLSEAANRNNDALRQAKQESTEYRRQVQSLTCEVDALKGTNESLERQMR |
| | | EMEENFAVEAANYQDTIGRLQDEIQNMKEEMARHLREYQDLLNVKMALDIEIATYRKL |
| | | LEGEESRISLPLPNFSSLNLRETNLDSLPLVDTHSKRTLLIKTVETRDGQVINETSQHHD |
| | | DLE |
| 40 | Human HSP90A | MPEETQTQDQPMEEEEVETFAFQAEIAQLMSLIINTFYSNKEIFLRELISNSSDALDKIR |
| | isoform 1 | YESLTDPSKLDSGKELHINLIPNKQDRTLTIVDTGIGMTKADLINNLGTIAKSGTKAFMEA |
| | (UniProt | LQAGADISMIGQFGVGFYSAYLVAEKVTVITKHNDDEQYAWESSAGGSFTVRTDTGE |
| | P07900-1) | PMGRGTKVILHLKEDQTEYLEERRIKEIVKKHSQFIGYPITLFVEKERDKEVSDDEAEEK |
| | | EDKEEEKEKEEKESEDKPEIEDVGSDEEEEKKDGDKKKKKKIKEKYIDQEELNKTKPIW |
| | | TRNPDDITNEEYGEFYKSLTNDWEDHLAVKHFSVEGQLEFRALLFVPRRAPFDLFENR |
| | | KKKNNIKLYVRRVFIMDNCEELIPEYLNFIRGVVDSEDLPLNISREMLQQSKILKVIRKNL |
| | | VKKCLELFTELAEDKENYKKFYEQFSKNIKLGIHEDSQNRKKLSELLRYYTSASGDEMV |
| | | SLKDYCTRMKENQKHIYYITGETKDQVANSAFVERLRKHGLEVIYMIEPIDEYCVQQLK |
| | | EFEGKTLVSVTKEGLELPEDEEEKKKQEEKKTKFENLCKIMKDILEKKVEKVVVSNRLV |
| | | TSPCCIVTSTYGWTANMERIMKAQALRDNSTMGYMAAKKHLEINPDHSIIETLRQKAE |
| | | ADKNDKSVKDLVILLYETALLSSGFSLEDPQTHANRIYRMIKLGLGIDEDDPTADDTSAA |
| | | VTEEMPPLEGDDDTSRMEEVD |
| 41 | Human HSP90A | MPPCSGGDGSTPPGPSLRDRDCPAQSAEYPRDRLDPRPGSPSEASSPPFLRSRAPV |
| | isoform 2 | NWYQEKAQVFLWHLMVSGSTTLLCLWKQPFHVSAFPVTASLAFRQSQGAGQHLYKD |
| | (UniProt | LQPFILLRLLMPEETQTQDQPMEEEEVETFAFQAEIAQLMSLIINTFYSNKEIFLRELISN |
| | P07900-2) | SSDALDKIRYESLTDPSKLDSGKELHINLIPNKQDRTLTIVDTGIGMTKADLINNLGTIAK |
| | | SGTKAFMEALQAGADISMIGQFGVGFYSAYLVAEKVTVITKHNDDEQYAWESSAGGS |
| | | FTVRTDTGEPMGRGTKVILHLKEDQTEYLEERRIKEIVKKHSQFIGYPITLFVEKERDKE |
| | | VSDDEAEEKEDKEEEKEKEEKESEDKPEIEDVGSDEEEEKKDGDKKKKKKIKEKYIDQ |
| | | EELNKTKPIWTRNPDDITNEEYGEFYKSLTNDWEDHLAVKHFSVEGQLEFRALLFVPR |
| | | RAPFDLFENRKKKNNIKLYVRRVFIMDNCEELIPEYLNFIRGVVDSEDLPLNISREMLQQ |
| | | SKILKVIRKNLVKKCLELFTELAEDKENYKKFYEQFSKNIKLGIHEDSQNRKKLSELLRY |
| | | YTSASGDEMVSLKDYCTRMKENQKHIYYITGETKDQVANSAFVERLRKHGLEVIYMIE |
| | | PIDEYCVQQLKEFEGKTLVSVTKEGLELPEDEEEKKKQEEKKTKFENLCKIMKDILEKK |
| | | VEKVVVSNRLVTSPCCIVTSTYGWTANMERIMKAQALRDNSTMGYMAAKKHLEINPD |
| | | HSIIETLRQKAEADKNDKSVKDLVILLYETALLSSGFSLEDPQTHANRIYRMIKLGLGIDE |
| | | DDPTADDTSAAVTEEMPPLEGDDDTSRMEEVD |
| 42 | Human HSP9OB | MPEEVHHGEEEVETFAFQAEIAQLMSLIINTFYSNKEIFLRELISNASDALDKIRYESLTD |
| | (UniProt | PSKLDSGKELKIDIIPNPQERTLTLVDTGIGMTKADLINNLGTIAKSGTKAFMEALQAGA |
| | P08238) | DISMIGQFGVGFYSAYLVAEKVVVITKHNDDEQYAWESSAGGSFTVRADHGEPIGRGT |
| | | KVILHLKEDQTEYLEERRVKEVVKKHSQFIGYPITLYLEKEREKEISDDEAEEEKGEKEE |
| | | EDKDDEEKPKIEDVGSDEEDDSGKDKKKKTKKIKEKYIDQEELNKTKPIWTRNPDDITQ |
| | | EEYGEFYKSLTNDWEDHLAVKHFSVEGQLEFRALLFIPRRAPFDLFENKKKKNNIKLYV |
| | | RRVFIMDSCDELIPEYLNFIRGVVDSEDLPLNISREMLQQSKILKVIRKNIVKKCLELFSE |
| | | LAEDKENYKKFYEAFSKNLKLGIHEDSTNRRRLSELLRYHTSQSGDEMTSLSEYVSRM |
| | | KETQKSIYYITGESKEQVANSAFVERVRKRGFEVVYMTEPIDEYCVQQLKEFDGKSLV |
| | | SVTKEGLELPEDEEEKKKMEESKAKFENLCKLMKEILDKKVEKVTISNRLVSSPCCIVT |
| | | STYGWTANMERIMKAQALRDNSTMGYMMAKKHLEINPDHPIVETLRQKAEADKNDKA |
| | | VKDLVVLLFETALLSSGFSLEDPQTHSNRIYRMIKLGLGIDEDEVAAEEPNAAVPDEIPP |
| | | LEGDEDASRMEEVD |
| 43 | Human | MMDQARSAFSNLFGGEPLSYTRFSLARQVDGDNSHVEMKLAVDEEENADNNTKANV |
| | Transferrin | TKPKRCSGSICYGTIAVIVFFLIGFMIGYLGYCKGVEPKTECERLAGTESPVREEPGED |
| | Receptor 1 | FPAARRLYWDDLKRKLSEKLDSTDFTGTIKLLNENSYVPREAGSQKDENLALYVENQF |
| | (UniProt | REFKLSKVWRDQHFVKIQVKDSAQNSVIIVDKNGRLVYLVENPGGYVAYSKAATVTGK |
| | P02786) | LVHANFGTKKDFEDLYTPVNGSIVIVRAGKITFAEKVANAESLNAIGVLIYMDQTKFPIVN |
| | | AELSFFGHAHLGTGDPYTPGFPSFNHTQFPPSRSSGLPNIPVQTISRAAAEKLFGNME |
| | | GDCPSDWKTDSTCRMVTSESKNVKLTVSNVLKEIKILNIFGVIKGFVEPDHYVVVGAQ |
| | | RDAWGPGAAKSGVGTALLLKLAQMFSDMVLKDGFQPSRSIIFASWSAGDFGSVGATE |
| | | WLEGYLSSLHLKAFTYINLDKAVLGTSNFKVSASPLLYTLIEKTMQNVKHPVTGQFLYQ |
| | | DSNWASKVEKLTLDNAAFPFLAYSGIPAVSFCFCEDTDYPYLGTTMDTYKELIERIPEL |
| | | NKVARAAAEVAGQFVIKLTHDVELNLDYERYNSQLLSFVRDLNQYRADIKEMGLSLQW |
| | | LYSARGDFFRATSRLTTDFGNAEKTDRFVMKKLNDRVMRVEYHFLSPYVSPKESPFR |
| | | HVFWGSGSHTLPALLENLKLRKQNNGAFNETLFRNQLALATWTIQGAANALSGDVWD |
| | | IDNEF |
| 44 | Human | MERLWGLFQRAQQLSPRSSQTVYQRVEGPRKGHLEEEEEDGEEGAETLAHFCPMEL |
| | Transferrin | RGPEPLGSRPRQPNLIPWAAAGRRAAPYLVLTALLIFTGAFLLGYVAFRGSCQACGDS |
| | Receptor 2 | VLVVSEDVNYEPDLDFHQGRLYWSDLQAMFLQFLGEGRLEDTIRQTSLRERVAGSAG |
| | isoform α | MAALTQDIRAALSRQKLDHVWTDTHYVGLQFPDPAHPNTLHWVDEAGKVGEQLPLE |
| | (UniProt | DPDVYCPYSAIGNVTGELVYAHYGRPEDLQDLRARGVDPVGRLLLVRVGVISFAQKVT |
| | Q9UP52-1) | NAQDFGAQGVLIYPEPADFSQDPPKPSLSSQQAVYGHVHLGTGDPYTPGFPSFNQT |
| | | QFPPVASSGLPSIPAQPISADIASRLLRKLKGPVAPQEWQGSLLGSPYHLGPGPRLRL |
| | | VVNNHRTSTPINNIFGCIEGRSEPDHYVVIGAQRDAWGPGAAKSAVGTAILLELVRTFS |
| | | SMVSNGFRPRRSLLFISWDGGDFGSVGSTEWLEGYLSVLHLKAVVYVSLDNAVLGDD |
| | | KFHAKTSPLLTSLIESVLKQVDSPNHSGQTLYEQVVFTNPSWDAEVIRPLPMDSSAYS |
| | | FTAFVGVPAVEFSFMEDDQAYPFLHTKEDTYENLHKVLQGRLPAVAQAVAQLAGQLLI |
| | | RLSHDRLLPLDFGRYGDVVLRHIGNLNEFSGDLKARGLTLQWVYSARGDYIRAAEKLR |
| | | QEIYSSEERDERLTRMYNVRIMRVEFYFLSQYVSPADSPFRHIFMGRGDHTLGALLDH |
| | | LRLLRSNSSGTPGATSSTGFQESRFRRQLALLTWTLQGAANALSGDVWNIDNNF |
| 45 | Human | MAALTQDIRAALSRQKLDHVWTDTHYVGLQFPDPAHPNTLHWVDEAGKVGEQLPLE |
| | Transferrin | DPDVYCPYSAIGNVTGELVYAHYGRPEDLQDLRARGVDPVGRLLLVRVGVISFAQKVT |
| | Receptor 2 | NAQDFGAQGVLIYPEPADFSQDPPKPSLSSQQAVYGHVHLGTGDPYTPGFPSFNQT |
| | isoform β | QFPPVASSGLPSIPAQPISADIASRLLRKLKGPVAPQEWQGSLLGSPYHLGPGPRLRL |
| | (UniProt | VVNNHRTSTPINNIFGCIEGRSEPDHYVVIGAQRDAWGPGAAKSAVGTAILLELVRTFS |
| | Q9UP52-2) | SMVSNGFRPRRSLLFISWDGGDFGSVGSTEWLEGYLSVLHLKAVVYVSLDNAVLGDD |
| | | KFHAKTSPLLTSLIESVLKQVDSPNHSGQTLYEQVVFTNPSWDAEVIRPLPMDSSAYS |
| | | FTAFVGVPAVEFSFMEDDQAYPFLHTKEDTYENLHKVLQGRLPAVAQAVAQLAGQLLI |
| | | RLSHDRLLPLDFGRYGDVVLRHIGNLNEFSGDLKARGLTLQWVYSARGDYIRAAEKLR |
| | | QEIYSSEERDERLTRMYNVRIMRVEFYFLSQYVSPADSPFRHIFMGRGDHTLGALLDH |
| | | LRLLRSNSSGTPGATSSTGFQESRFRRQLALLTWTLQGAANALSGDVWNIDNNF |
| 46 | Human | MERLWGLFQRAQQLSPRSSQTVYQRVEGPRKGHLEEEEEDGEEGAETLAHFCPMEL |
| | Transferrin | RGPEPLGSRPRQPNLIPWAAAGRRAAPYLVLTALLIFTGAFLLGYVAFRGSCQACGDS |
| | Receptor 2 | VLVVSEDVNYEPDLDFHQGRLYWSDLQAMFLQFLGEGRLEDTIRQTSLRERVAGSAG |
| | isoform γ | MAALTQDIRAALSRQKLDHVWTDTHYVGLQFPDPAHPNTLHWVDEAGKVGEQLPLE |
| | (UniProt | DPDVYCPYSAIGNVTGELVYAHYGRPEDLQDLRARGVDPVGRLLLVRVGVISFAQKVT |
| | Q9UP52-3) | NAQDFGAQGVLIYPEPADFSQDPPKPSLSSQQAVYGHVHLGTGDPYTPGFPSFNQT |
| | | QKLKGPVAPQEWQGSLLGSPYHLGPGPRLRLVVNNHRTSTPINNIFGCIEGRSEPDH |
| | | YVVIGAQRDAWGPGAAKSAVGTAILLELVRTFSSMVSNGFRPRRSLLFISWDGGDFG |
| | | SVGSTEWLEGYLSVLHLKAVVYVSLDNAVLGDDKFHAKTSPLLTSLIESVLKQVDSPN |
| | | HSGQTLYEQVVFTNPSWDAEVIRPLPMDSSAYSFTAFVGVPAVEFSFMEDDQAYPFL |
| | | HTKEDTYENLHKVLQGRLPAVAQAVAQLAGQLLIRLSHDRLLPLDFGRYGDVVLRHIG |
| | | NLNEFSGDLKARGLTLQWVYSARGDYIRAAEKLRQEIYSSEERDERLTRMYNVRIMRV |
| | | EFYFLSQYVSPADSPFRHIFMGRGDHTLGALLDHLRLLRSNSSGTPGATSSTGFQESR |
| | | FRRQLALLTWTLQGAANALSGDVWNIDNNF |
| 47 | Human PGFR-a | MGTSHPAFLVLGCLLTGLSLILCQLSLPSILPNENEKVVQLNSSFSLRCFGESEVSWQY |
| | isoform 1 | PMSEEESSDVEIRNEENNSGLFVTVLEVSSASAAHTGLYTCYYNHTQTEENELEGRHI |
| | (UniProt | YIYVPDPDVAFVPLGMTDYLVIVEDDDSAIIPCRTTDPETPVTLHNSEGVVPASYDSRQ |
| | P16234-1) | GFNGTFTVGPYICEATVKGKKFQTIPFNVYALKATSELDLEMEALKTVYKSGETIVVTC |
| | | AVFNNEVVDLQWTYPGEVKGKGITMLEEIKVPSIKLVYTLTVPEATVKDSGDYECAAR |
| | | QATREVKEMKKVTISVHEKGFIEIKPTFSQLEAVNLHEVKHFVVEVRAYPPPRISWLKN |
| | | NLTLIENLTEITTDVEKIQEIRYRSKLKLIRAKEEDSGHYTIVAQNEDAVKSYTFELLTQV |
| | | PSSILDLVDDHHGSTGGQTVRCTAEGTPLPDIEWMICKDIKKCNNETSWTILANNVSNII |
| | | TEIHSRDRSTVEGRVTFAKVEETIAVRCLAKNLLGAENRELKLVAPTLRSELTVAAAVL |
| | | VLLVIVIISLIVLVVIWKQKPRYEIRWRVIESISPDGHEYIYVDPMQLPYDSRWEFPRDGL |
| | | VLGRVLGSGAFGKVVEGTAYGLSRSQPVMKVAVKMLKPTARSSEKQALMSELKIMTH |
| | | LGPHLNIVNLLGACTKSGPIYIITEYCFYGDLVNYLHKNRDSFLSHHPEKPKKELDIFGL |
| | | NPADESTRSYVILSFENNGDYMDMKQADTTQYVPMLERKEVSKYSDIQRSLYDRPAS |
| | | YKKKSMLDSEVKNLLSDDNSEGLTLLDLLSFTYQVARGMEFLASKNCVHRDLAARNVL |
| | | LAQGKIVKICDFGLARDIMHDSNYVSKGSTFLPVKWMAPESIFDNLYTTLSDVWSYGIL |
| | | LWEIFSLGGTPYPGMMVDSTFYNKIKSGYRMAKPDHATSEVYEIMVKCWNSEPEKRP |
| | | SFYHLSEIVENLLPGQYKKSYEKIHLDFLKSDHPAVARMRVDSDNAYIGVTYKNEEDKL |
| | | KDWEGGLDEQRLSADSGYIIPLPDIDPVPEEEDLGKRNRHSSQTSEESAIETGSSSST |
| | | FIKREDETIEDIDMMDDIGIDSSDLVEDSFL |
| 48 | Human PGFR-a | QLSLPSILPNENEKVVQLNSSFSLRCFGESEVSWQYPMSEEESSDVEIRNEENNSGLF |
| | isoform 1 | VTVLEVSSASAAHTGLYTCYYNHTQTEENELEGRHIYIYVPDPDVAFVPLGMTDYLVIV |
| | (mature | EDDDSAIIPCRTTDPETPVTLHNSEGVVPASYDSRQGFNGTFTVGPYICEATVKGKKF |
| | sequence, | QTIPFNVYALKATSELDLEMEALKTVYKSGETIVVTCAVFNNEVVDLQWTYPGEVKGK |
| | lacking signal | GITMLEEIKVPSIKLVYTLTVPEATVKDSGDYECAARQATREVKEMKKVTISVHEKGFIEI |
| | peptide; UniProt | KPTFSQLEAVNLHEVKHFVVEVRAYPPPRISWLKNNLTLIENLTEITTDVEKIQEIRYRS |
| | P16234-1 | KLKLIRAKEEDSGHYTIVAQNEDAVKSYTFELLTQVPSSILDLVDDHHGSTGGQTVRCT |
| | residues 24 to | AEGTPLPDIEWMICKDIKKCNNETSWTILANNVSNIITEIHSRDRSTVEGRVTFAKVEETI |
| | 1089) | AVRCLAKNLLGAENRELKLVAPTLRSELTVAAAVLVLLVIVIISLIVLVVIWKQKPRYEIR |
| | | WRVIESISPDGHEYIYVDPMQLPYDSRWEFPRDGLVLGRVLGSGAFGKVVEGTAYGL |
| | | SRSQPVMKVAVKMLKPTARSSEKQALMSELKIMTHLGPHLNIVNLLGACTKSGPIYIITE |
| | | YCFYGDLVNYLHKNRDSFLSHHPEKPKKELDIFGLNPADESTRSYVILSFENNGDYMD |
| | | MKQADTTQYVPMLERKEVSKYSDIQRSLYDRPASYKKKSMLDSEVKNLLSDDNSEGL |
| | | TLLDLLSFTYQVARGMEFLASKNCVHRDLAARNVLLAQGKIVKICDFGLARDIMHDSNY |
| | | VSKGSTFLPVKWMAPESIFDNLYTTLSDVWSYGILLWEIFSLGGTPYPGMMVDSTFYN |
| | | KIKSGYRMAKPDHATSEVYEIMVKCWNSEPEKRPSFYHLSEIVENLLPGQYKKSYEKI |
| | | HLDFLKSDHPAVARMRVDSDNAYIGVTYKNEEDKLKDWEGGLDEQRLSADSGYIIPLP |
| | | DIDPVPEEEDLGKRNRHSSQTSEESAIETGSSSSTFIKREDETIEDIDMMDDIGIDSSDL |
| | | VEDSFL |
| 49 | Human PGFR-a | QLSLPSILPNENEKVVQLNSSFSLRCFGESEVSWQYPMSEEESSDVEIRNEENNSGLF |
| | isoform 1 ECD | VTVLEVSSASAAHTGLYTCYYNHTQTEENELEGRHIYIYVPDPDVAFVPLGMTDYLVIV |
| | (UniProt | EDDDSAIIPCRTTDPETPVTLHNSEGVVPASYDSRQGFNGTFTVGPYICEATVKGKKF |
| | P16234-1 | QTIPFNVYALKATSELDLEMEALKTVYKSGETIVVTCAVFNNEVVDLQWTYPGEVKGK |
| | residues 24 | GITMLEEIKVPSIKLVYTLTVPEATVKDSGDYECAARQATREVKEMKKVTISVHEKGFIEI |
| | to 528) | KPTFSQLEAVNLHEVKHFVVEVRAYPPPRISWLKNNLTLIENLTEITTDVEKIQEIRYRS |
| | | KLKLIRAKEEDSGHYTIVAQNEDAVKSYTFELLTQVPSSILDLVDDHHGSTGGQTVRCT |
| | | AEGTPLPDIEWMICKDIKKCNNETSWTILANNVSNIITEIHSRDRSTVEGRVTFAKVEETI |
| | | AVRCLAKNLLGAENRELKLVAPTLRSELTVA |
| 50 | Human PGFR-a | MGTSHPAFLVLGCLLTGLSLILCQLSLPSILPNENEKVVQLNSSFSLRCFGESEVSWQY |
| | isoform 2 | PMSEEESSDVEIRNEENNSGLFVTVLEVSSASAAHTGLYTCYYNHTQTEENELEGRHI |
| | (UniProt | YIYVPDPDVAFVPLGMTDYLVIVEDDDSAIIPCRTTDPETPVTLHNSEGVVPASYDSRQ |
| | P16234-2) | GFNGTFTVGPYICEATVKGKKFQTIPFNVYALKGTCIISFLL |
| 51 | Human PGFR-a | QLSLPSILPNENEKVVQLNSSFSLRCFGESEVSWQYPMSEEESSDVEIRNEENNSGLF |
| | isoform 2 | VTVLEVSSASAAHTGLYTCYYNHTQTEENELEGRHIYIYVPDPDVAFVPLGMTDYLVIV |
| | (mature | EDDDSAIIPCRTTDPETPVTLHNSEGVVPASYDSRQGFNGTFTVGPYICEATVKGKKF |
| | sequence, | QTIPFNVYALKGTCIISFLL |
| | lacking signal | |
| | peptide; UniProt | |
| | P16234-2 | |
| | residues 24 to | |
| | 218) | |
| 52 | Human PGFR-a | MGTSHPAFLVLGCLLTGLSLILCQLSLPSILPNENEKVVQLNSSFSLRCFGESEVSWQY |
| | isoform 3 | PMSEEESSDVEIRNEENNSGLFVTVLEVSSASAAHTGLYTCYYNHTQTEENELEGRHI |
| | (UniProt | YIYVPDPDVAFVPLGMTDYLVIVEDDDSAIIPCRTTDPETPVTLHNSEGVVPASYDSRQ |
| | P16234-3) | GFNGTFTVGPYICEATVKGKKFQTIPFNVYALKATSELDLEMEALKTVYKSGETIVVTC |
| | | AVFNNEVVDLQWTYPGEVKGKGITMLEEIKVPSIKLVYTLTVPEATVKDSGDYECAAR |
| | | QATREVKEMKKVTISVHEKGFIEIKPTFSQLEAVNLHEVKHFVVEVRAYPPPRISWLKN |
| | | NLTLIENLTEITTDVEKIQEIRYRSKLKLIRAKEEDSGHYTIVAQNEDAVKSYTFELLTQV |
| | | PSSILDLVDDHHGSTGGQTVRCTAEGTPLPDIEWMICKDIKKCNNETSWTILANNVSNII |
| | | TEIHSRDRSTVEGRVTFAKVEETIAVRCLAKNLLGAENRELKLVAPTLRSELTVAAAVL |
| | | VLLVIVIISLIVLVVIWKQKPRYEIRWRVIESISPDGHEYIYVDPMQLPYDSRWEFPRDGL |
| | | VLGRVLGSGAFGKVVEGTAYGLSRSQPVMKVAVKMLKPTARSSEKQALMSELKIMTH |
| | | LGPHLNIVNLLGACTKSGPIYIITEYCFYGDLVNYLHKNRDSFLSHHPEKPKKELDIFGL |
| | | NPADESTRSSGQGCLSSGTLQELSVDLQARGPC |
| 53 | Human PGFR-a | QLSLPSILPNENEKVVQLNSSFSLRCFGESEVSWQYPMSEEESSDVEIRNEENNSGLF |
| | isoform 3 | VTVLEVSSASAAHTGLYTCYYNHTQTEENELEGRHIYIYVPDPDVAFVPLGMTDYLVIV |
| | (mature | EDDDSAIIPCRTTDPETPVTLHNSEGVVPASYDSRQGFNGTFTVGPYICEATVKGKKF |
| | sequence, | QTIPFNVYALKATSELDLEMEALKTVYKSGETIVVTCAVFNNEVVDLQWTYPGEVKGK |
| | lacking signal | GITMLEEIKVPSIKLVYTLTVPEATVKDSGDYECAARQATREVKEMKKVTISVHEKGFIEI |
| | peptide; UniProt | KPTFSQLEAVNLHEVKHFVVEVRAYPPPRISWLKNNLTLIENLTEITTDVEKIQEIRYRS |
| | P16234-3 | KLKLIRAKEEDSGHYTIVAQNEDAVKSYTFELLTQVPSSILDLVDDHHGSTGGQTVRCT |
| | residues 24 to | AEGTPLPDIEWMICKDIKKCNNETSWTILANNVSNIITEIHSRDRSTVEGRVTFAKVEETI |
| | 743) | AVRCLAKNLLGAENRELKLVAPTLRSELTVAAAVLVLLVIVIISLIVLVVIWKQKPRYEIR |
| | | WRVIESISPDGHEYIYVDPMQLPYDSRWEFPRDGLVLGRVLGSGAFGKVVEGTAYGL |
| | | SRSQPVMKVAVKMLKPTARSSEKQALMSELKIMTHLGPHLNIVNLLGACTKSGPIYIITE |
| | | YCFYGDLVNYLHKNRDSFLSHHPEKPKKELDIFGLNPADESTRSSGQGCLSSGTLQEL |
| | | SVDLQARGPC |
| 54 | Human CD3ε | MQSGTHWRVLGLCLLSVGVWGQDGNEEMGGITQTPYKVSISGTTVILTCPQYPGSEI |
| | (UniProt | LWQHNDKNIGGDEDDKNIGSDEDHLSLKEFSELEQSGYYVCYPRGSKPEDANFYLYL |
| | P07766) | RARVCENCMEMDVMSVATIVIVDICITGGLLLLVYYWSKNRKAKAKPVTRGAGAGGRQ |
| | | RGQNKERPPPVPNPDYEPIRKGQRDLYSGLNQRRI |
| 55 | Human CD3ε | DGNEEMGGITQTPYKVSISGTTVILTCPQYPGSEILWQHNDKNIGGDEDDKNIGSDED |
| | (mature | HLSLKEFSELEQSGYYVCYPRGSKPEDANFYLYLRARVCENCMEMDVMSVATIVIVDI |
| | sequence, | CITGGLLLLVYYWSKNRKAKAKPVTRGAGAGGRQRGQNKERPPPVPNPDYEPIRKG |
| | lacking signal | QRDLYSGLNQRRI |
| | peptide; UniProt | |
| | P07766 | |
| | residues 23 to | |
| | 207) | |
| 56 | Human CD3ε | DGNEEMGGITQTPYKVSISGTTVILTCPQYPGSEILWQHNDKNIGGDEDDKNIGSDED |
| | ECD (UniProt | HLSLKEFSELEQSGYYVCYPRGSKPEDANFYLYLRARVCENCMEMD |
| | P007766 | |
| | residues 23 to | |
| | 126) | |
| 57 | Human CCR5 | MDYQVSSPIYDINYYTSEPCQKINVKQIAARLLPPLYSLVFIFGFVGNMLVILILINCKRLK |
| | (UniProt | SMTDIYLLNLAISDLFFLLTVPFWAHYAAAQWDFGNTMCQLLTGLYFIGFFSGIFFIILLTI |
| | P51681) | DRYLAVVHAVFALKARTVTFGVVTSVITWVVAVFASLPGIIFTRSQKEGLHYTCSSHFP |
| | | YSQYQFWKNFQTLKIVILGLVLPLLVMVICYSGILKTLLRCRNEKKRHRAVRLIFTIMIVY |
| | | FLFWAPYNIVLLLNTFQEFFGLNNCSSSNRLDQAMQVTETLGMTHCCINPIIYAFVGEK |
| | | FRNYLLVFFQKHIAKRFCKCCSIFQQEAPERASSVYTRSTGEQEISVGL |
| 58 | Truncated P15 | AGACAAGAAUAAACGCUCAAAGUUG |
| | (vimentin- | |
| | binding) | |
| 59 | Truncated TR14 | UUUAUUCACAUUUUUGAAUUGA |
| | (TfR-binding) | |
| | 22mer | |
| 60 | Human CEA | GLHNHHPIKPFITSNNSNPVEDEDAVALTCEPEIQNTTYLWWVNNQSLPVSPRLQLSN |
| | (UniProt | DNRTLTLLSVTRNDVGPYECGIQNELSVDHSDPVILNVLYGPDDPTISPSYTYYRPGVN |
| | Q13984) | LSLSCHAASNPPAQYSWLIDGNIQQHTQELFISNITEKNSGLYTCQANNSASGHSRTT |
| | | VKTITVSADVPKPSISSNNSKPVEDKDAVAFHCEPEAQNTTYLWWVNGQSLPVSPRL |
| | | QLSNGNRTLTLFNVTRNDARAYVCGIQNSVSANRSDPVTLDVLYGPDTPIISPPP |
| 61 | Truncated | GUUCGUAGCGAUUUAACUGCGUCAGC |
| | CD3e3 | |

Provided herein includes the combination of the aspects and preferred features described except where such a combination is clearly impermissible or expressly avoided.

The section headings used herein are for organizational purposes only and are not to be construed as limiting the subject matter described.

Aspects and embodiments described herein will now be illustrated, by way of example, with reference to the accompanying figures. Further aspects and embodiments will be apparent to those skilled in the art.

All documents mentioned in this text are incorporated herein by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Where a nucleic acid sequence is disclosed herein, the reverse complement thereof is also expressly contemplated.

### BRIEF DESCRIPTION OF THE FIGURES

Embodiments and experiments illustrating the principles will now be discussed with reference to the accompanying figures.

FIG. 1. Schematic representation of chimeric aptamer/antibody bispecific T cell engager (BiTE) molecule comprising mortalin binding aptamer tP19, and anti-human CD3ε antibody OKT3. The molecule is designated chBiTE.

FIGS. 2A to 2D. Graphs showing binding of different molecules to PANC-1 cells, as determined by flow cytometry analysis. FIG. 2A shows a negative control, where cells were stained with DAPI only.

FIG. 2B shows a further control condition, where cells were incubated with anti-CD3 antibody. FIG. 2C shows a positive control condition, where cells were incubated with the mortalin-binding aptamer tP19.

FIG. 2D shows the result when cells were incubated with chBiTE.

FIGS. 3A to 3D. Graphs showing binding of different molecules to human T cells, as determined by flow cytometry analysis. FIG. 3A shows a negative control, where cells were stained with DAPI only.

FIG. 3B shows a further control condition, where cells were incubated with secondary antibody only.

FIG. 3C shows a positive control condition, where cells were incubated with anti-CD3 antibody. FIG. 3D shows the result when cells were incubated with chBiTE.

FIGS. 4A to 4D. Images obtained by fluorescence confocal microscopy of live cells showing localisation of chBiTE bound to PANC-1 cells. FIG. 4A shows localisation of chBiTE. FIG. 4B shows nucleic acid staining by Hoechest 33342. FIG. 4C shows a bright field image. FIG. 4D is a merge of the signals from FIGS. 4A and 4B with FIG. 4C.

FIGS. 5A to 5C. Bar charts and graph showing results of analysis of the ability of different molecules to potentiate cell killing of different cell types by cytotoxic T lymphocytes, as determined by LDH release assay. FIG. 5A shows killing of PANC-1 (mortalin positive) cells and BJ (mortalin negative) cells by CTLs in the presence of chBiTE, anti-CD3 antibody or mortalin-binding aptamer tP19. FIG. 5B shows killing of PANC-1 cells by CTLs in the presence of different concentrations of chBiTE, anti-CD3 antibody or tP19. FIG. 5C shows killing of U251 cells and HCT116 cells by CTLs in the presence of chBiTE, anti-CD3 antibody or tP19.

FIG. 6. Graph showing results of analysis of the ability of different chBiTE, isotype control (ISP) antibody or tP19 to potentiate cell killing of PANC-1 cells by cytotoxic T lymphocytes, as determined by LDH release assay.

### EMBODIMENTS

Embodiments include but are not limited to embodiments P1 to P31 following:

### Embodiment P1

An antigen-binding molecule capable of binding to a cancer cell antigen and an immune cell surface molecule, comprising: (i) an aptamer, and (ii) an antigen-binding polypeptide.

### Embodiment P2

The antigen-binding molecule according to embodiment P1, which is capable of binding to cells expressing the cancer cell antigen at the cell surface.

### Embodiment P3

The antigen-binding molecule according to embodiment P1 or embodiment P2, which is capable of increasing killing of cells expressing the cancer cell antigen at the cell surface by an immune cell.

### Embodiment P4

The antigen-binding molecule according to any one of embodiments P1 to P3, wherein the cancer cell antigen is selected from the group consisting of: mortalin, vimentin, HSP90, TfR, PDGFR-a and CEA.

### Embodiment P5

The antigen-binding molecule according to any one of embodiments P1 to P4, wherein the immune cell surface molecule is selected from the group consisting of: a CD3-TCR complex polypeptide, CD3ε, CD3γ, CD3δ, CD3ζ, CD3η, TCRα, TCRβ, TCRγ, TCRδ, CD27, CD28, CD4, CD8, CCR5, CCR7, CD2, CD7, PD-1, and CTLA4.

### Embodiment P6

The antigen-binding molecule according to any one of embodiments P1 to P5, wherein the aptamer is a cancer cell antigen-binding aptamer.

### Embodiment P7

The antigen-binding molecule according to embodiment P6, wherein the cancer cell antigen-binding aptamer comprises, or consists of, a nucleic acid sequence having at least 80% sequence identity to one of SEQ ID NOs:1 to 13, 58 and 59.

### Embodiment P8

The antigen-binding molecule according to embodiment P6 or embodiment P7, wherein the cancer cell antigen is mortalin.

### Embodiment P9

The antigen-binding molecule according to any one of embodiments P6 to P8, wherein the cancer cell antigen-binding aptamer comprises, or consists of, a nucleic acid sequence having at least 80% sequence identity to one of SEQ ID NOs:1, 2, 3 and 8.

### Embodiment P10

The antigen-binding molecule according to any one of embodiments P1 to P9, wherein the antigen-binding polypeptide is an immune cell surface molecule-binding polypeptide.

### Embodiment P11

The antigen-binding molecule according to embodiment P10, wherein the immune cell surface molecule-binding polypeptide comprises a CD3ε-binding domain comprising the amino acid sequences i) to vi):

(SEQ ID NO: 26)
i) LC-CDR1: RASSSVSYMN
(SEQ ID NO: 27)
ii) LC-CDR2: DTSKVAS
(SEQ ID NO: 28)
iii) LC-CDR3: QQWSSNPLT
(SEQ ID NO: 30)
iv) HC-CDR1: RYTMH
(SEQ ID NO: 31)
v) HC-CDR2: YINPSRGYTNYNQKFKD
(SEQ ID NO: 32)
vi) HC-CDR3: YYDDHYCLDY;

or a variant thereof in which one or two or three amino acids in one or more of the sequences i) to vi) are replaced with another amino acid.

### Embodiment P12

The antigen-binding molecule according to embodiment P10 or embodiment P11, wherein the immune cell surface molecule-binding polypeptide comprises a CD3ε-binding domain comprising:
a variable light chain (VL) region comprising, or consisting of, an amino acid sequence having at least 70% sequence identity to the amino acid sequence of SEQ ID NO:25; and
a variable heavy chain (VH) region comprising, or consisting of, an amino acid sequence having at least 70% sequence identity to the amino acid sequence of SEQ ID NO:29.

### Embodiment P13

The antigen-binding molecule according to any one of embodiments P1 to P12, wherein the antigen-binding polypeptide is a cancer cell antigen-binding polypeptide.

### Embodiment P14

The antigen-binding molecule according to embodiment P13, wherein the cancer cell antigen-binding polypeptide comprises a mortalin-binding domain comprising the amino acid sequences i) to vi):

(SEQ ID NO: 18)
i) LC-CDR1: KSSQSLLDSDGKTYLN
(SEQ ID NO: 19)
ii) LC-CDR2: LVSKLDS
(SEQ ID NO: 20)
iii) LC-CDR3: WQGTHFPRT
(SEQ ID NO: 22)
iv) HC-CDR1: SYWMH
(SEQ ID NO: 23)
v) HC-CDR2: EIDPSDSYTKYNQKFKG
(SEQ ID NO: 24)
vi) HC-CDR3: GDY;

or a variant thereof in which one or two or three amino acids in one or more of the sequences i) to vi) are replaced with another amino acid.

### Embodiment P15

The antigen-binding molecule according to embodiment P13 or embodiment P14, wherein the immune cell surface molecule-binding polypeptide comprises a mortalin-binding domain comprising:
a variable light chain (VL) region comprising, or consisting of, an amino acid sequence having at least 70% sequence identity to the amino acid sequence of SEQ ID NO:17; and
a variable heavy chain (VH) region comprising, or consisting of, an amino acid sequence having at least 70% sequence identity to the amino acid sequence of SEQ ID NO:21.

### Embodiment P16

The antigen-binding molecule according to any one of embodiments P1 to P15, wherein the aptamer is an immune cell surface molecule-binding aptamer.

### Embodiment P17

The antigen-binding molecule according to embodiment P16, wherein the immune cell surface molecule-binding aptamer comprises, or consists of, a nucleic acid sequence having at least 80% sequence identity to one of SEQ ID NOs:14 to 16 and 61.

### Embodiment P18

An antigen-binding molecule capable of binding to a cancer cell antigen and an immune cell surface molecule, comprising: (i) a cancer cell antigen-binding aptamer, and (ii) an immune cell surface molecule-binding polypeptide.

### Embodiment P19

The antigen-binding molecule according to embodiment P18, which comprises a mortalin-binding aptamer and a CD3ε-binding polypeptide.

### Embodiment P20

The antigen-binding molecule according to embodiment P19, wherein the mortalin-binding aptamer comprises, or consists of, a nucleic acid sequence having at least 80% sequence identity to one of SEQ ID NOs:1, 2, 3 and 8.

### Embodiment P21

The antigen-binding molecule according to embodiment P19 or embodiment P20, wherein the CD3ε-binding polypeptide comprises a CD3ε-binding domain comprising:
a variable light chain (VL) region comprising, or consisting of, an amino acid sequence having at least 70% sequence identity to the amino acid sequence of SEQ ID NO:27; and
a variable heavy chain (VH) region comprising, or consisting of, an amino acid sequence having at least 70% sequence identity to the amino acid sequence of SEQ ID NO:31.

### Embodiment P22

The antigen-binding molecule according to any one of embodiments P1 to P21, wherein the aptamer and antigen-binding polypeptide are covalently associated with one another.

### Embodiment P23

A complex, optionally an in vitro complex, comprising the antigen-binding molecule according to any one of embodiments P1 to P22 bound to a cancer cell antigen and/or an immune cell surface molecule.

### Embodiment P24

A composition comprising the antigen-binding molecule according to any one of embodiments P1 to P22 and at least one pharmaceutically-acceptable carrier, diluent or excipient.

### Embodiment P25

The antigen-binding molecule of any one of embodiments P1 to P22, or the composition of embodiment P24, for use in therapy, or in a method of medical treatment.

### Embodiment P26

The antigen-binding molecule of any one of embodiments P1 to P22, or the composition of embodiment P24, for use in the treatment or prevention of a cancer.

### Embodiment P27

Use of the antigen-binding molecule of any one of embodiments P1 to P22, or the composition of embodiment P24, in the manufacture of a medicament for treating or preventing a cancer.

### Embodiment P28

A method of treating or preventing a cancer, comprising administering to a subject a therapeutically or prophylactically effective amount of the antigen-binding molecule of any one of embodiments P1 to P22, or the composition of embodiment P24.

### Embodiment P29

A method of diagnosing or prognosing a cancer in a subject, comprising contacting a sample from the subject with an antigen-binding molecule according to any one of embodiments P1 to P22, and detecting binding of the antigen-binding molecule to a cancer cell antigen and/or an immune cell surface molecule.

### Embodiment P30

The antigen-binding molecule or composition for use according to embodiment P26, the use according to embodiment P27, or the method according to embodiment P28 or embodiment P29, wherein the cancer is selected from the group consisting of: pancreatic cancer, brain cancer, colorectal cancer, liver cancer, breast cancer and gastric cancer.

### Embodiment P31

A kit of parts comprising a predetermined quantity of the antigen-binding molecule of any one of embodiments P1 to P22, or the composition of embodiment P24.

### EXAMPLES

In the following Examples the inventors describe the design and functional characterisation of a chimeric, bispecific antigen-binding molecule comprising a CD3-binding polypeptide moiety and a mortalin-binding aptamer moiety.

### Example 1: Construction of Chimeric Aptamer/Antibody Molecule

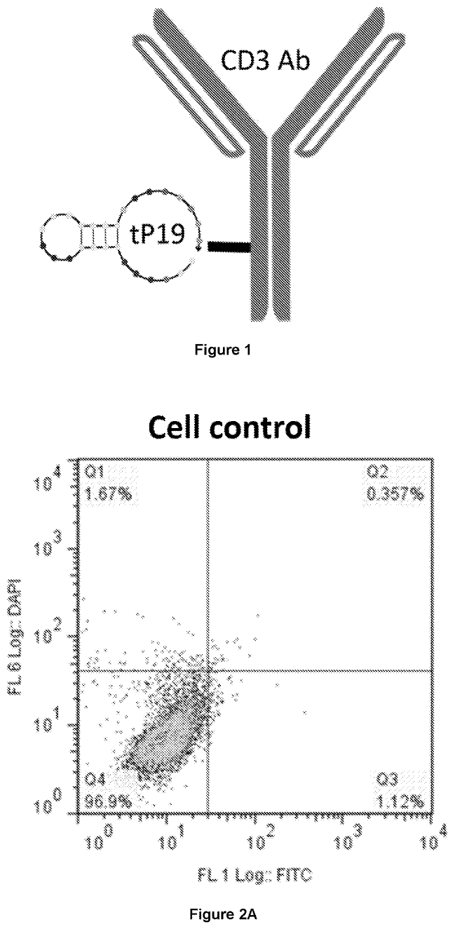

Truncated mortalin aptamer (tP19; SEQ ID NO:1) was chemically conjugated to mouse anti-human CD3ε antibody OKT3 to construct a chimeric aptamer/antibody bispecific T cell engager (BiTE) molecule, hereafter referred to as “chBiTE” (FIG. 1).

Anti-CD3-CH₂CO-DIBOA was prepared as follows. 2 mg (220 μl, 13.33 nmol) of anti-CD3 antibody was added to 180 ul PBS, and reduced by a 30 molar excess of TCEP. TCEP was removed using a Zeba column, the reduced antibody was then reacted with Br—CH₂CO-DIBOA (20 molar excess) at pH 7.5, room temperature under Argon, overnight.

The anti-CD3-tP19 conjugate (chBiTE) was then prepared as follows. 2 mg of anti-CD3-CH₂CO-DIBOA was mixed with tP19 (13.33 nmol, pH7.3). The then mixture was then rotated under Argon at room temperature for 1 h, and then incubated overnight at 4° C.

ChBiTE was purified using a Superdex 200 column. The peak at 24.5 min was collected, and subsequently concentrated.

### Example 2: Analysis of Binding of chBiTE to T Cells and Pancreatic Cancer Cells

Analysis of binding of chBiTE to T cells and pancreatic cancer cells (PANC-1) was analysed by flow cytometry.

Analysis of Binding to Pancreatic Cancer Cells:

PANC-1 cells were suspended with Accutase and incubated at 1×10⁵ cells/mL with 10 μg/mL of chBiTE for 1 hour on ice, washed, and incubated with anti-mouse Alexa-488 antibody (Thermo Fisher Scientific, Waltham, Mass.). Negative controls included (i) untreated cells, except for DAPI stain (‘cell control’) and (ii) cells incubated with mouse anti-human CD3 antibody OKT3 (InVivoMab, Bio X cell) for 1 hour on ice, followed by washing and incubation with anti-mouse Alexa-488 antibody (‘CD3 Ab’ control). As a positive control, cells were incubated with Alexa-488 labeled anti-mortalin aptamer tP19. Before flow cytometry analysis, DAPI (1 μg/mL) was added to all samples to allow exclusion of dead cells.

Analysis of Binding to Human T Cells:

Human T cells were isolated by negative selection using the EasySep human T cell isolation kit (STEMCELL Technologies). Freshly isolated human T cells (1×10⁵ cells/mL) were incubated with 10 μg/mL of chBiTE for 1 hour on ice, washed, and then incubated with anti-mouse Alexa-488 antibody (Thermo Fisher Scientific, Waltham, Mass.). Negative controls included (i) untreated cells, except for DAPI stain (‘cell control’) and (ii) cells treated with anti-mouse Alexa-488 antibody only (‘secondary antibody control’). As a positive control, cells were incubated with mouse anti-human CD3 antibody OKT3 (InVivoMab, Bio X cell) for 1 hour on ice, followed by washing and incubation with anti-mouse Alexa-488 antibody. Before flow cytometry analysis, DAPI (1 μg/mL) was added to all samples to allow exclusion of dead cells.

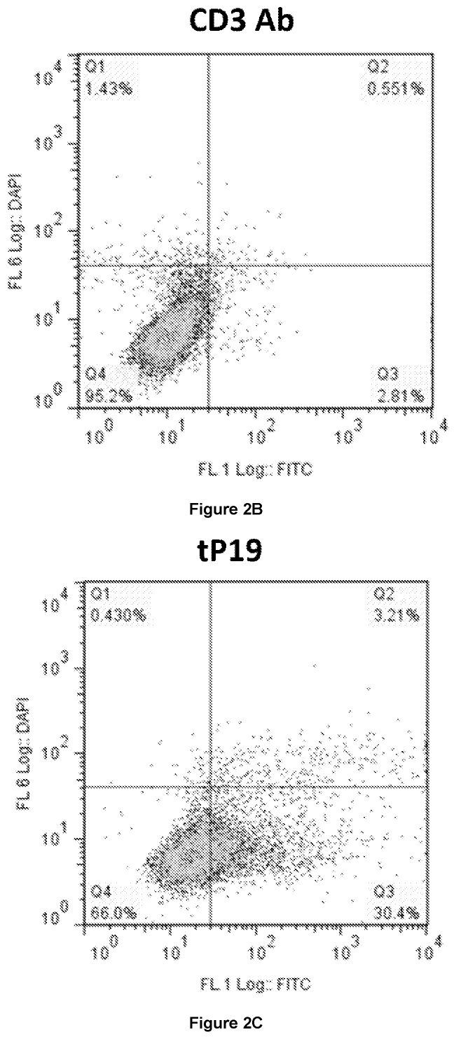

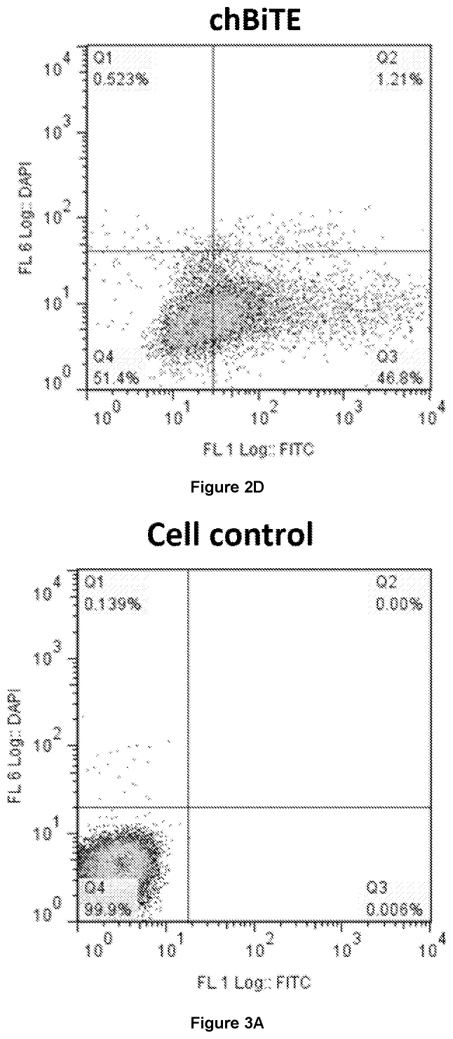

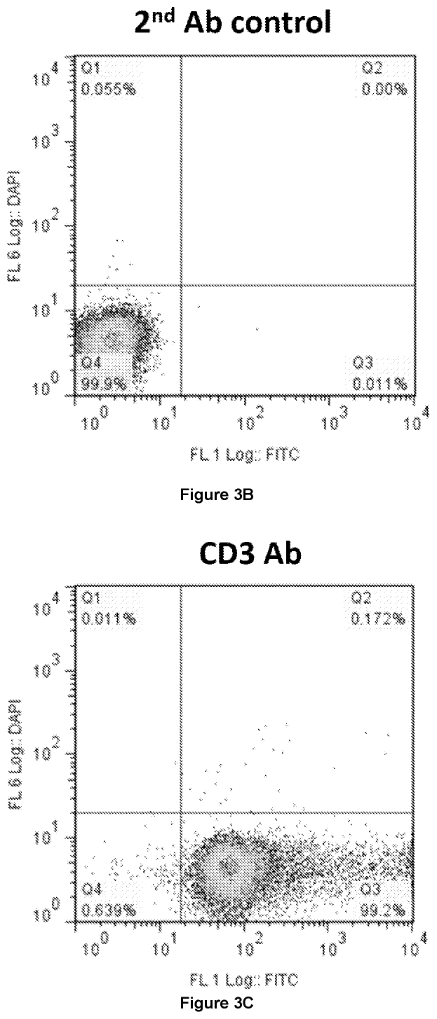

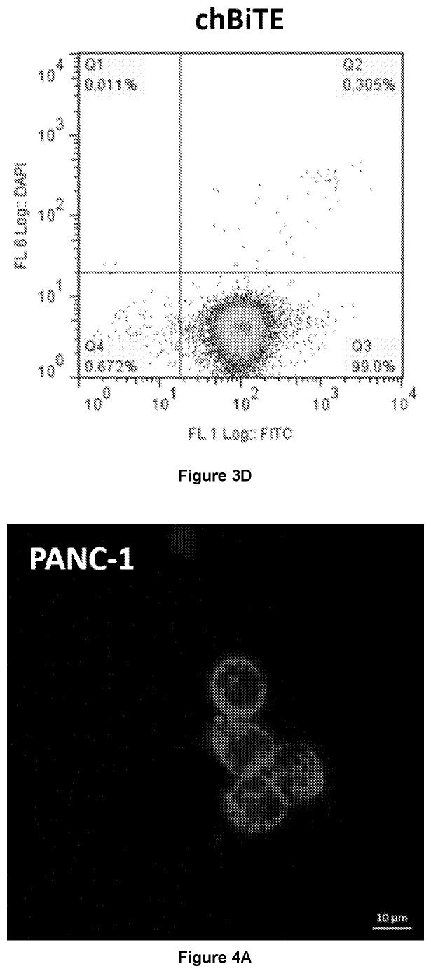

The results are shown in FIGS. 2 and 3. Compared to controls, chBiTE showed binding to 46.8% of PANC-1 cells (FIG. 2D), and to 99.0% of human T cells (FIG. 3D).

### Example 3: Analysis of Internalisation of chBiTE by Pancreatic Cancer Cells

Binding and internalisation of chBiTE by live PANC-1 cells was analysed by confocal microscopy.

PANC-1 cells at 1×10⁵ cells/mL were seeded in 35-mm glass-bottom dishes one day before the analysis. Cells were incubated with chBiTE (2 μg/ml) at 37° C., followed by washing and incubation with anti-mouse Alexa-555 antibody (Thermo Fisher Scientific, Waltham, Mass.). After washing, cells were incubated at 37° C. for 6 hours before live cell imaging by fluorescence confocal microscopy (Carl Zeiss, Jena, Germany).

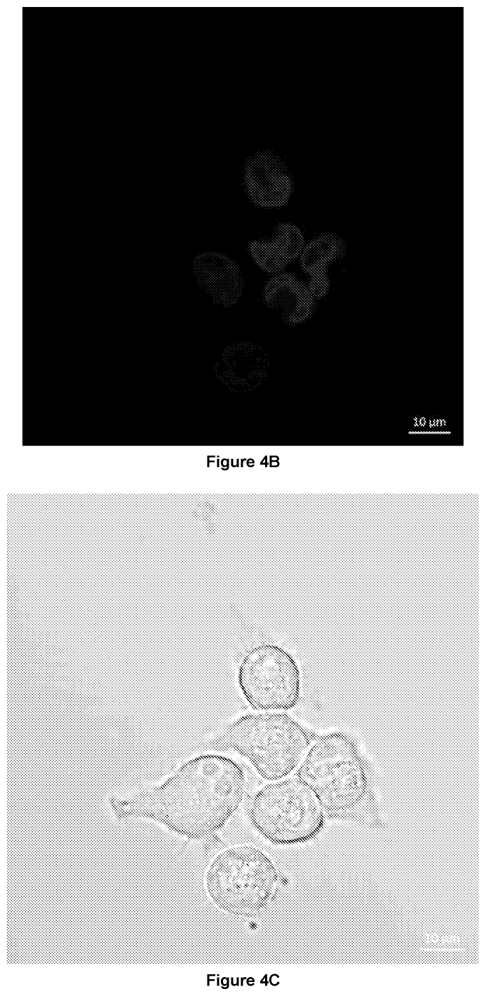

The results of the experiment are shown in FIG. 4. ChBiTE was determined mostly to remain on the cell surface of PANC-1 cells, and not to be internalised.

### Example 4: Analysis of Influence of chBiTE CTL Killing of Mortalin-Expressing Cells

The influence of chBiTE on cell killing of mortalin-expressing cells by cytotoxic T lymphocytes (CTLs) was analysed using an in vitro assay of CTL activity.

Analysis where Target Cells were Pre-Incubated with chBiTE:

Target human cancer cells (PANC-1 cells, U251 cells or HCT116 cells) were seeded at 1×10⁴ cells/well one day before the CTL assay. The target cells were then pre-incubated with chBiTE or negative control reagents at various concentrations at room temperature for 30 minutes. Negative control reagents included mouse anti-human CD3 antibody OKT3 and tP19 aptamer. Human PBMCs, freshly isolated using SepMate (STEMCELL technologies), were added at an effector cell-to-target cell ratio (E/T ratio) of 10:1 (based on total PBMC cell count), and cells incubated overnight at 37° C. Cytotoxic activity was measured by lactate dehydrogenase (LDH) release assay using the Cytotox 96 non-radioactive kit from Promega, according to the manufacturer's instructions.

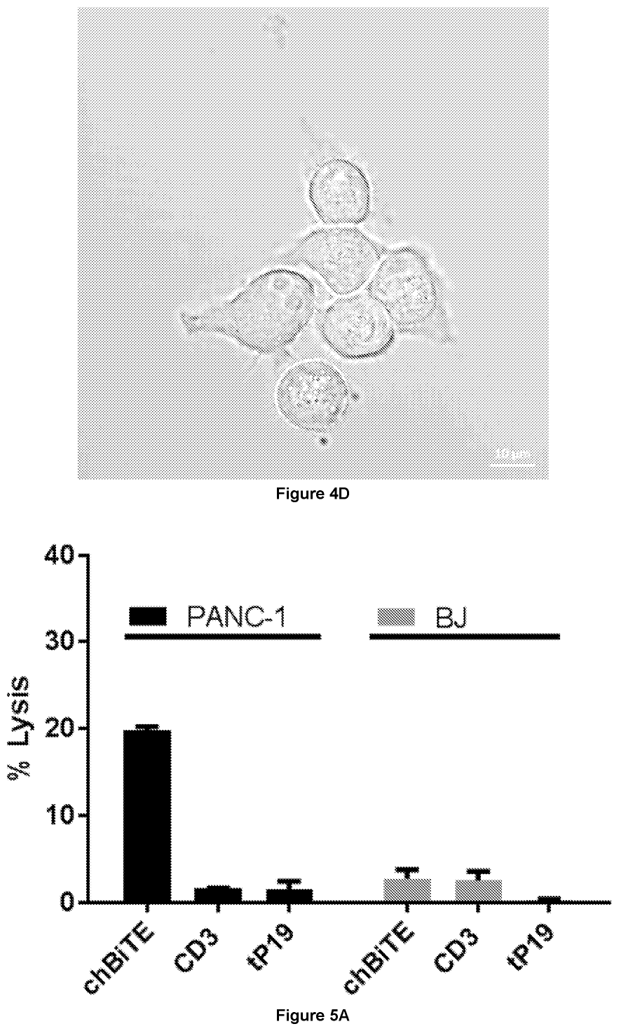

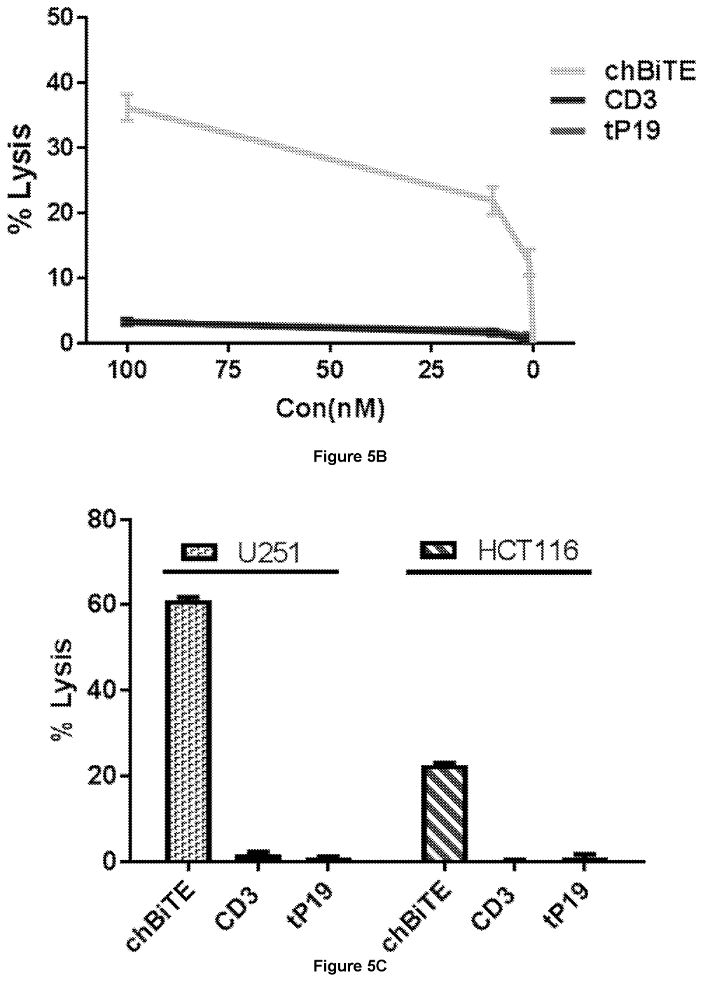

The results of the experiment are shown in FIG. 5. ChBiTE was found to potentiate killing of PANC-1 cells to a level of cell killing of 20%, whilst no significant killing was of mortalin-negative BJ cells was detected (FIG. 5A). ChBiTE was found to promote T cell cytotoxicity against PANC-1 cells up to a level of cell killing of 35.5%, in a dose-dependent manner (FIG. 5B). When the T-cell cytotoxicity was assessed on other mortalin-positive cancer cell types, including glioblastoma cells (U251) and colon cancer cells (HCT116), levels of cell killing of 61% and 22% were detected, respectively (FIG. 5C).

Analysis where T Cells were Pre-Incubated with chBiTE:

Human PBMCs, freshly isolated using SepMate (STEMCELL technologies), were pre-incubated with chBiTE or negative control reagents at various concentrations at room temperature for 30 minutes. Target PANC-1 cells were added at an effector cell-to-target cell ratio (E/T ratio) of 10:1 (based on total PBMC cell count), and cells incubated overnight at 37° C. Cytotoxic activity was measured by lactate dehydrogenase (LDH) release assay using the Cytotox 96 non-radioactive kit from Promega, according to the manufacturer's instructions.

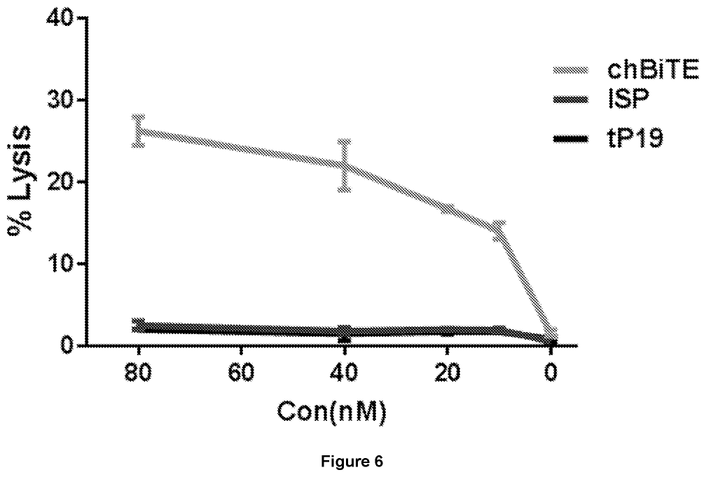

The results of the experiment are shown in FIG. 6. ChBiTE was found to promote T cell cytotoxicity against PANC-1 cells in a dose-dependent manner (FIG. 6).

1. An antigen-binding molecule capable of binding to a cancer cell antigen and an immune cell surface molecule, comprising: (i) an aptamer, and (ii) an antigen-binding polypeptide.

2. The antigen-binding molecule according to claim 1, which is capable of binding to cells expressing the cancer cell antigen at the cell surface.

3. The antigen-binding molecule according to claim 2, which is capable of increasing killing of cells expressing the cancer cell antigen at the cell surface by an immune cell.

4. The antigen-binding molecule according to claim 1, wherein the cancer cell antigen is selected from the group consisting of: mortalin, vimentin, HSP90, TfR, PDGFR-a and CEA.

5. The antigen-binding molecule according to claim 1, wherein the immune cell surface molecule is selected from the group consisting of: a CD3-TCR complex polypeptide, CD3ε, CD3γ, CD3δ, CD3ζ CD3η, TCRα, TCRβ, TCRγ, TCRδ, CD27, CD28, CD4, CD8, CCR5, CCR7, CD2, CD7, PD-1, and CTLA4.

6. The antigen-binding molecule according to claim 1, wherein the aptamer is a cancer cell antigen-binding aptamer.

7. The antigen-binding molecule according to claim 6, wherein the cancer cell antigen-binding aptamer comprises, or consists of, a nucleic acid sequence having at least 80% sequence identity to one of SEQ ID NOs:1 to 13, 58 and 59.

8. The antigen-binding molecule according to claim 6, wherein the cancer cell antigen is mortalin.

9. The antigen-binding molecule according to claim 6, wherein the cancer cell antigen-binding aptamer comprises, or consists of, a nucleic acid sequence having at least 80% sequence identity to one of SEQ ID NOs:1, 2, 3 and 8.

10. The antigen-binding molecule according to claim 1, wherein the antigen-binding polypeptide is an immune cell surface molecule-binding polypeptide.

11. The antigen-binding molecule according to claim 10, wherein the immune cell surface molecule-binding polypeptide comprises a CD3ε-binding domain comprising the amino acid sequences i) to vi):
(SEQ ID NO: 26)
i) LC-CDR1: RASSSVSYMN
(SEQ ID NO: 27)
ii) LC-CDR2: DTSKVAS
(SEQ ID NO: 28)
iii) LC-CDR3: QQWSSNPLT
(SEQ ID NO: 30)
iv) HC-CDR1: RYTMH
(SEQ ID NO: 31)
v) HC-CDR2: YINPSRGYTNYNQKFKD
(SEQ ID NO: 32)
vi) HC-CDR3: YYDDHYCLDY;
or a variant thereof in which one or two or three amino acids in one or more of the sequences i) to vi) are replaced with another amino acid.

12. The antigen-binding molecule according to claim 10, wherein the immune cell surface molecule-binding polypeptide comprises a CD3ε-binding domain comprising:
a variable light chain (VL) region comprising, or consisting of, an amino acid sequence having at least 70% sequence identity to the amino acid sequence of SEQ ID NO:25; and
a variable heavy chain (VH) region comprising, or consisting of, an amino acid sequence having at least 70% sequence identity to the amino acid sequence of SEQ ID NO:29.

13. The antigen-binding molecule according to claim 1, wherein the antigen-binding polypeptide is a cancer cell antigen-binding polypeptide.

14. The antigen-binding molecule according to claim 13, wherein the cancer cell antigen-binding polypeptide comprises a mortalin-binding domain comprising the amino acid sequences i) to vi):
(SEQ ID NO: 18)
i) LC-CDR1: KSSQSLLDSDGKTYLN
(SEQ ID NO: 19)
ii) LC-CDR2: LVSKLDS
(SEQ ID NO: 20)
iii) LC-CDR3: WQGTHFPRT
(SEQ ID NO: 22)
iv) HC-CDR1: SYVVMH
(SEQ ID NO: 23)
v) HC-CDR2: EIDPSDSYTKYNQKFKG
(SEQ ID NO: 24)
vi) HC-CDR3: GDY;
or a variant thereof in which one or two or three amino acids in one or more of the sequences i) to vi) are replaced with another amino acid.

15. The antigen-binding molecule according to claim 10, wherein the immune cell surface molecule-binding polypeptide comprises a mortalin-binding domain comprising:
a variable light chain (VL) region comprising, or consisting of, an amino acid sequence having at least 70% sequence identity to the amino acid sequence of SEQ ID NO:17; and
a variable heavy chain (VH) region comprising, or consisting of, an amino acid sequence having at least 70% sequence identity to the amino acid sequence of SEQ ID NO:21.

16. The antigen-binding molecule according to claim 1, wherein the aptamer is an immune cell surface molecule-binding aptamer.

17. The antigen-binding molecule according to claim 16, wherein the immune cell surface molecule-binding aptamer comprises, or consists of, a nucleic acid sequence having at least 80% sequence identity to one of SEQ ID NOs:14 to 16 and 61.

18. An antigen-binding molecule capable of binding to a cancer cell antigen and an immune cell surface molecule, comprising: (i) a cancer cell antigen-binding aptamer, and (ii) an immune cell surface molecule-binding polypeptide.

19. The antigen-binding molecule according to claim 18, which comprises a mortalin-binding aptamer and a CD3ε-binding polypeptide.

20. The antigen-binding molecule according to claim 19, wherein the mortalin-binding aptamer comprises, or consists of, a nucleic acid sequence having at least 80% sequence identity to one of SEQ ID NOs:1, 2, 3 and 8.

21. The antigen-binding molecule according to claim 19, wherein the CD3ε-binding polypeptide comprises a CD3ε-binding domain comprising:
a variable light chain (VL) region comprising, or consisting of, an amino acid sequence having at least 70% sequence identity to the amino acid sequence of SEQ ID NO:27; and
a variable heavy chain (VH) region comprising, or consisting of, an amino acid sequence having at least 70% sequence identity to the amino acid sequence of SEQ ID NO:31.

22. The antigen-binding molecule according to claim 1, wherein the aptamer and antigen-binding polypeptide are covalently associated with one another.

23. A complex, optionally an in vitro complex, comprising the antigen-binding molecule according to any one of claims 1 to 22 bound to a cancer cell antigen and/or an immune cell surface molecule.

24. A composition comprising the antigen-binding molecule according to any one of claims 1 to 22 and at least one pharmaceutically-acceptable carrier, diluent or excipient.

25. The antigen-binding molecule of any one of claims 1 to 22, or the composition of claim 24, for use in therapy, or in a method of medical treatment.

26. The antigen-binding molecule of any one of claims 1 to 22, or the composition of claim 24, for use in the treatment or prevention of a cancer.

27. Use of the antigen-binding molecule of any one of claims 1 to 22, or the composition of claim 24, in the manufacture of a medicament for treating or preventing a cancer.

28. A method of treating or preventing a cancer, comprising administering to a subject a therapeutically or prophylactically effective amount of the antigen-binding molecule of any one of claims 1 to 22, or the composition of claim 24.

29. A method of diagnosing or prognosing a cancer in a subject, comprising contacting a sample from the subject with an antigen-binding molecule according to any one of claims 1 to 22, and detecting binding of the antigen-binding molecule to a cancer cell antigen and/or an immune cell surface molecule.

30. The antigen-binding molecule or composition for use according to claim 26, the use according to claim 27, or the method according to claim 28 or claim 29, wherein the cancer is selected from the group consisting of: pancreatic cancer, brain cancer, colorectal cancer, liver cancer, breast cancer and gastric cancer.

31. A kit of parts comprising a predetermined quantity of the antigen-binding molecule of any one of claims 1 to 22, or the composition of claim 24.
